(19) United States
(12) Patent Application Publication
(10) Pub. No.: US 2024/0182553 A1
(43) Pub. Date: Jun. 6, 2024

(54) ANTIBODY FUSION PROTEINS AND USES THEREOF

(71) Applicant: Nanjing GenScript Biotech Co., Ltd., Nanjing (CN)

(72) Inventors: Zhongdao Li, Nanjing (CN); Yi Jin, Nanjing (CN); Wenshuang Jia, Nanjing (CN); Weiming Wang, Nanjing (CN); Zhihui Zhao, Nanjing (CN); Brian Hosung Min, Nanjing (CN); Cuiying Shao, Nanjing (CN)

(73) Assignee: Nanjing GenScript Biotech Co., Ltd., Nanjing, JS (CN)

(21) Appl. No.: 18/281,849

(22) PCT Filed: Mar. 23, 2022
(86) PCT No.: PCT/CN2022/082448

§ 371 Date: Sep. 13, 2023

(30) Foreign Application Priority Data
Mar. 25, 2021 (WO) PCT/CN2021/083093

(51) Int. Cl. C07K 16/22; A61K 38/00; A61K 39/00; C07K 14/71

(52) U.S. Cl. CPC C07K 16/22; C07K 14/71; A61K 38/00; A61K 39/00; C07K 2317/35; C07K 2319/30

(57) ABSTRACT

The present application provides an antibody fusion protein comprising: i) a multivalent (e.g., bivalent) antibody or antigen-binding fragment thereof specifically recognizing Angiopoietin-2 (Ang2)(“multivalent anti-Ang2 antibody or antigen-binding fragment thereof”), and ii) a vascular endothelial growth factor receptor (VEGFR) component, wherein the multivalent anti-Ang2 antibody or antigen-binding fragment thereof does not inhibit the binding between Ang2 and TEK receptor tyrosine kinase (TIE2). Also provided are methods of making and uses thereof.

### CROSS REFERENCE TO RELATED APPLICATIONS

This application claims priority benefits of International Patent Application No. PCT/CN2021/083093 filed Mar. 25, 2021, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 759892000940SEQLIST.TXT, date recorded: Jan. 19, 2022, size: 223 KB).

### FIELD OF THE INVENTION

The present application provides an antibody fusion protein comprising: i) a multivalent (e.g., bivalent) antibody or antigen-binding fragment thereof specifically recognizing Angiopoietin-2 (Ang2)(“multivalent anti-Ang2 antibody or antigen-binding fragment thereof”), and ii) a vascular endothelial growth factor receptor (VEGFR) component, wherein the multivalent anti-Ang2 antibody or antigen-binding fragment thereof does not inhibit the binding between Ang2 and TEK receptor tyrosine kinase (Tie2). Also provided are methods of making and uses thereof.

### BACKGROUND OF THE INVENTION

Maintaining endothelial barrier function is critical for vascular normalization that involves several physiological processes including angiogenesis, and its dysfunction will lead to several cancers and non-neoplastic diseases including rheumatoid arthritis, atherosclerosis, diabetic retinopathy, age-related macular degeneration (AMD), as well as infections like sepsis, dengue fever, and malaria.

Physiological angiogenesis occurs during reproduction, tissue remodeling. In contrast, the neovascularization that occurs under pathological conditions leads to capillary leakage followed by tissue damage. The underlying molecular mechanisms behind pathological capillary leakage is under intensive studies. Angiogenesis is a complex process that is driven by interactions between extracellular matrix-derived angiogenic inhibitors and growth factors including vascular endothelial growth factor (VEGF), fibroblast growth factor (FGF), insulin-like growth factor (IGF), and angiopoietins.

VEGF has been the most widely exploited target for antiangiogenic therapy. The binding of VEGF to its tyrosine kinase receptors (VEGFR) expressed on vascular endothelial cells triggers cellular responses involved in vascular endothelial cell proliferation and new blood vessel growth. Recombinant fusion protein comprising VEGFR components has been developed to treat diseases associated with VEGF expression, such as cancer and AMD. These fusion proteins are generated by fusing VEGFR fragments to the N-terminus of an immunoglobulin Fc fragment, or a portion of the immunoglobulin Fc fragment, and are recognized as “VEGF-trap.” See, e.g. U.S. Pat. Nos. 6,100,071, 7,087,411, and 7,521,049. The VEGFR fragments will bind to and inactivate endogenous VEGF, thereby providing a means for reducing or inhibiting endogenous VEGF activity and, in turn, reducing or inhibiting endothelial cell proliferation and angiogenesis. The VEGF-trap (Aflibercept) can capture VEGF with higher affinity (˜0.49 pM) than bevacizumab (e.g., Avastin®, VEGF neutralizing antibody, ˜58 pM), brolucizumab (DLX1008, ˜1 pM), and ranibizumab (Lucentis®, affinity ˜46 pM). However, for treatment of retinal vascular disease, anti-VEGF treatments, while effective in a significant number of patients, still do not prevent the progression to legal blindness in many patients.

Tie2 is an endothelial cell-specific receptor tyrosine kinase (RTK) that is oppositely modulated by two secreted proteins, angiopoietin-1 (ANGPT-1, Ang1) and angiopoietin-2 (ANGPT-2, Ang2). Ang1 acts as Tie2 agonist leading to its phosphorylation inducing various protective downstream pathways, which induces the stabilization of vascular endothelial cells and reduces vascular permeability by accelerating the junctional integrity of the vascular endothelial cells. In contrast, Ang2 is an endogenous, context-specific antagonist/agonist of Tie2 by competing with Ang1 as an antagonist or by converting to a potent Tie2 activator. Ang2 plays important roles in physiological processes and the mis-regulation of its expression is characteristic of several diseases.

Currently, a variety of drugs targeting Ang2 are under clinical studies. Most of the Ang2-targeting drugs (e.g., anti-Ang2 antagonist antibody) block Ang2 binding to Tie2. In the context of several diseases or infections like sepsis, Ang2 expression is increased, whereas Ang1 and Tie2 expression declines. Therefore, simply blocking Ang2-Tie2 interaction is unable to maintain vascular normalization.

Further, Ang2 promotes the proangiogenic action of VEGF, and VEGF upregulates Ang2 expression in endothelial cells (A. Hegen et al., Arterioscler Thromb Vasc Biol. 2004; 24(10):1803-1809; H. Hashizume et al., Cancer Res. 2010; 70(6):2213-222), targeting either VEGF or Ang2 can only provide transient or modest benefit.

The disclosures of all publications, patents, patent applications and published patent applications referred to herein are hereby incorporated herein by reference in their entirety.

### BRIEF SUMMARY OF THE INVENTION

One aspect of the present application provides an antibody fusion protein (hereinafter also referred to as “anti-Ang2/VEGF-trap antibody fusion protein” or “anti-Ang2/VEGFR antibody fusion protein”) comprising: i) a multivalent (e.g., bivalent) anti-Ang2 antibody (e.g., full length antibody) or antigen-binding fragment thereof, and ii) a VEGFR component, wherein the multivalent anti-Ang2 antibody or antigen-binding fragment thereof does not inhibit the binding between Ang2 and Tie2. In some embodiments, the multivalent anti-Ang2 antibody or antigen-binding fragment thereof upon binding to Ang2 activates Tie2 signaling through the antibody-bound Ang2. In some embodiments, the VEGFR component comprises an immunoglobulin (Ig)-like domain of one or more VEGFRs independently selected from the group consisting of Flt1, Flk1, and Flt4. In some embodiments, the VEGFR component comprises an Ig-like domain 2 of a first VEGFR and an Ig-like domain 3 of a second VEGFR. In some embodiments, the first VEGFR is Flt1, and the second VEGFR is Flk1 or Flt4. In some embodiments, the VEGFR component comprises an Ig-like domain 2 of Flt1 (Flt1d2) and an Ig-like domain 3 of Flk1 (Flk1d3). In some embodiments, the VEGFR component comprises an amino acid sequence of SEQ ID NO: 32. In some embodiments, the VEGFR component is fused to the multivalent anti-Ang2 antibody or antigen-binding fragment thereof via an optional linker, such as a linker comprising the amino acid sequence of SEQ ID NO: 30 or 31.

In some embodiments according to any of the antibody fusion proteins described above, the multivalent anti-Ang2 antibody or antigen-binding fragment thereof is a full length antibody (“anti-Ang2 full length antibody”). In some embodiments, the anti-Ang2 full length antibody comprises any of the following: (1) a heavy chain comprising the amino acid sequence of SEQ ID NO: 33 or 34, and a light chain comprising the amino acid sequence of any of SEQ ID NOs: 41-43; (2) a heavy chain comprising the amino acid sequence of SEQ ID NO: 35, and a light chain comprising the amino acid sequence of SEQ ID NO: 44 or 49; (3) a heavy chain comprising the amino acid sequence of SEQ ID NO: 36 or 37, and a light chain comprising the amino acid sequence of SEQ ID NO: 45 or 46; (4) a heavy chain comprising the amino acid sequence of SEQ ID NO: 38, and a light chain comprising the amino acid sequence of any of SEQ ID NOs: 41-43; (5) a heavy chain comprising the amino acid sequence of SEQ ID NO: 39, and a light chain comprising the amino acid sequence of SEQ ID NO: 47; or (6) a heavy chain comprising the amino acid sequence of SEQ ID NO: 40, and a light chain comprising the amino acid sequence of SEQ ID NO: 48. In some embodiments, the anti-Ang2 full length antibody comprises any of the following: (1) a heavy chain comprising the amino acid sequence of SEQ ID NO: 33, and a light chain comprising the amino acid sequence of SEQ ID NO: 42; (2) a heavy chain comprising the amino acid sequence of SEQ ID NO: 34, and a light chain comprising the amino acid sequence of any of SEQ ID NOs: 41-43; (3) a heavy chain comprising the amino acid sequence of SEQ ID NO: 35, and a light chain comprising the amino acid sequence of SEQ ID NO: 44; (4) a heavy chain comprising the amino acid sequence of SEQ ID NO: 36, and a light chain comprising the amino acid sequence of SEQ ID NO: 46; (5) a heavy chain comprising the amino acid sequence of SEQ ID NO: 37, and a light chain comprising the amino acid sequence of SEQ ID NO: 45; (6) a heavy chain comprising the amino acid sequence of SEQ ID NO: 38, and a light chain comprising the amino acid sequence of SEQ ID NO: 42; (7) a heavy chain comprising the amino acid sequence of SEQ ID NO: 39, and a light chain comprising the amino acid sequence of SEQ ID NO: 47; or (8) a heavy chain comprising the amino acid sequence of SEQ ID NO: 40, and a light chain comprising the amino acid sequence of SEQ ID NO: 48. In some embodiments, the anti-Ang2 full length antibody is monospecific. In some embodiments, the anti-Ang2 full length antibody is bispecific. In some embodiments, the VEGFR component is fused to the N-terminus of a heavy chain of the anti-Ang2 full length antibody via an optional linker. In some embodiments, the heavy chain fusion polypeptide comprises the amino acid sequence of any of SEQ ID NOs: 56-61, 75, and 79. In some embodiments, the antibody fusion protein comprises any of the following: (1) a heavy chain fusion polypeptide comprising the amino acid sequence of SEQ ID NO: 56 or 58, and a light chain comprising the amino acid sequence of SEQ ID NO: 42; (2) a heavy chain fusion polypeptide comprising the amino acid sequence of SEQ ID NO: 57, and a light chain comprising the amino acid sequence of SEQ ID NO: 41 or 42; (3) a heavy chain fusion polypeptide comprising the amino acid sequence of SEQ ID NO: 59, and a light chain comprising the amino acid sequence of SEQ ID NO: 45; (4) a heavy chain fusion polypeptide comprising the amino acid sequence of SEQ ID NO: 60, and a light chain comprising the amino acid sequence of SEQ ID NO: 46; (5) a heavy chain fusion polypeptide comprising the amino acid sequence of SEQ ID NO: 61, and a light chain comprising the amino acid sequence of SEQ ID NO: 44; (6) a heavy chain fusion polypeptide comprising the amino acid sequence of SEQ ID NO: 75, and a light chain comprising the amino acid sequence of SEQ ID NO: 47; or (7) a heavy chain fusion polypeptide comprising the amino acid sequence of SEQ ID NO: 79, and a light chain comprising the amino acid sequence of SEQ ID NO: 48. In some embodiments, the antibody fusion protein comprises a first VEGFR component and a second VEGFR component, wherein the first VEGFR component is fused to the N-terminus of a first heavy chain of the anti-Ang2 full length antibody via an optional first linker, and wherein the second VEGFR component is fused to the N-terminus of a second heavy chain of the anti-Ang2 full length antibody via an optional second linker. In some embodiments, the two heavy chain fusion polypeptides are the same. In some embodiments, the VEGFR component is fused to the C-terminus of a heavy chain of the anti-Ang2 full length antibody via an optional linker. In some embodiments, the heavy chain fusion polypeptide comprises the amino acid sequence of any of SEQ ID NOs: 50-55, 74, and 78. In some embodiments, the antibody fusion protein comprises any of the following: (1) a heavy chain fusion polypeptide comprising the amino acid sequence of SEQ ID NO: 50 or 52, and a light chain comprising the amino acid sequence of SEQ ID NO: 42; (2) a heavy chain fusion polypeptide comprising the amino acid sequence of SEQ ID NO: 51, and a light chain comprising the amino acid sequence of SEQ ID NO: 41 or 42; (3) a heavy chain fusion polypeptide comprising the amino acid sequence of SEQ ID NO: 53, and a light chain comprising the amino acid sequence of SEQ ID NO: 45; (4) a heavy chain fusion polypeptide comprising the amino acid sequence of SEQ ID NO: 54, and a light chain comprising the amino acid sequence of SEQ ID NO: 46; (5) a heavy chain fusion polypeptide comprising the amino acid sequence of SEQ ID NO: 55, and a light chain comprising the amino acid sequence of SEQ ID NO: 44; (6) a heavy chain fusion polypeptide comprising the amino acid sequence of SEQ ID NO: 74, and a light chain comprising the amino acid sequence of SEQ ID NO: 47; or (7) a heavy chain fusion polypeptide comprising the amino acid sequence of SEQ ID NO: 78, and a light chain comprising the amino acid sequence of SEQ ID NO: 48. In some embodiments, the antibody fusion protein comprises a first VEGFR component and a second VEGFR component, wherein the first VEGFR component is fused to the C-terminus of a first heavy chain of the anti-Ang2 full length antibody via an optional first linker, and wherein the second VEGFR component is fused to the C-terminus of a second heavy chain of the anti-Ang2 full length antibody via an optional second linker. In some embodiments, the two heavy chain fusion polypeptides are the same. In some embodiments, the VEGFR component is fused to the N-terminus of a light chain of the anti-Ang2 full length antibody via an optional linker. In some embodiments, the light chain fusion polypeptide comprises the amino acid sequence of any of SEQ ID NOs: 67-71, 73, 77, and 81. In some embodiments, the antibody fusion protein comprises any of the following: (1) a heavy chain comprising the amino acid sequence of SEQ ID NO: 33, and a light chain fusion polypeptide comprising the amino acid sequence of SEQ ID NO: 67; (2) a heavy chain comprising the amino acid sequence of SEQ ID NO: 34, and a light chain fusion polypeptide comprising the amino acid sequence of any of SEQ ID NOs: 67, 68, and 77; (3) a heavy chain comprising the amino acid sequence of SEQ ID NO: 37, and a light chain fusion polypeptide comprising the amino acid sequence of SEQ ID NO: 69; (4) a heavy chain comprising the amino acid sequence of SEQ ID NO: 36, and a light chain fusion polypeptide comprising the amino acid sequence of SEQ ID NO: 70; (5) a heavy chain comprising the amino acid sequence of SEQ ID NO: 35, and a light chain fusion polypeptide comprising the amino acid sequence of SEQ ID NO: 71; (6) a heavy chain comprising the amino acid sequence of SEQ ID NO: 39, and a light chain fusion polypeptide comprising the amino acid sequence of SEQ ID NO: 73; (7) a heavy chain comprising the amino acid sequence of SEQ ID NO: 38, and a light chain fusion polypeptide comprising the amino acid sequence of SEQ ID NO: 67; or (8) a heavy chain comprising the amino acid sequence of SEQ ID NO: 40, and a light chain fusion polypeptide comprising the amino acid sequence of SEQ ID NO: 81. In some embodiments, the antibody fusion protein comprises a first VEGFR component and a second VEGFR component, wherein the first VEGFR component is fused to the N-terminus of a first light chain of the anti-Ang2 full length antibody via an optional first linker, and wherein the second VEGFR component is fused to the N-terminus of a second light chain of the anti-Ang2 full length antibody via an optional second linker. In some embodiments, the two light chain fusion polypeptides are the same. In some embodiments, the VEGFR component is fused to the C-terminus of a light chain of the anti-Ang2 full length antibody via an optional linker. In some embodiments, the light chain fusion polypeptide comprises the amino acid sequence of any of SEQ ID NOs: 62-66, 72, 76, and 80. In some embodiments, the antibody fusion protein comprises any of the following: (1) a heavy chain comprising the amino acid sequence of SEQ ID NO: 33, and a light chain fusion polypeptide comprising the amino acid sequence of SEQ ID NO: 62; (2) a heavy chain comprising the amino acid sequence of SEQ ID NO: 34, and a light chain fusion polypeptide comprising the amino acid sequence of any of SEQ ID NOs: 62, 63, and 76; (3) a heavy chain comprising the amino acid sequence of SEQ ID NO: 37, and a light chain fusion polypeptide comprising the amino acid sequence of SEQ ID NO: 64; (4) a heavy chain comprising the amino acid sequence of SEQ ID NO: 36, and a light chain fusion polypeptide comprising the amino acid sequence of SEQ ID NO: 65; (5) a heavy chain comprising the amino acid sequence of SEQ ID NO: 35, and a light chain fusion polypeptide comprising the amino acid sequence of SEQ ID NO: 66; (6) a heavy chain comprising the amino acid sequence of SEQ ID NO: 39, and a light chain fusion polypeptide comprising the amino acid sequence of SEQ ID NO: 72; (7) a heavy chain comprising the amino acid sequence of SEQ ID NO: 38, and a light chain fusion polypeptide comprising the amino acid sequence of SEQ ID NO: 62; or (8) a heavy chain comprising the amino acid sequence of SEQ ID NO: 40, and a light chain fusion polypeptide comprising the amino acid sequence of SEQ ID NO: 80. In some embodiments, the antibody fusion protein comprises a first VEGFR component and a second VEGFR component, wherein the first VEGFR component is fused to the C-terminus of a first light chain of the anti-Ang2 full length antibody via an optional first linker, and wherein the second VEGFR component is fused to the C-terminus of a second light chain of the anti-Ang2 full length antibody via an optional second linker. In some embodiments, the two light chain fusion polypeptides are the same. In some embodiments, the antibody fusion protein comprises four VEGFR components, wherein the antibody fusion protein comprises any of the following: (1) wherein the first VEGFR component is fused to the N-terminus of a first heavy chain of the anti-Ang2 full length antibody via an optional first linker, wherein the second VEGFR component is fused to the N-terminus of a second heavy chain of the anti-Ang2 full length antibody via an optional second linker, wherein the third VEGFR component is fused to the N-terminus of a first light chain of the anti-Ang2 full length antibody via an optional third linker, and wherein the fourth VEGFR component is fused to the N-terminus of a second light chain of the anti-Ang2 full length antibody via an optional fourth linker; (2) wherein the first VEGFR component is fused to the C-terminus of a first heavy chain of the anti-Ang2 full length antibody via an optional first linker, wherein the second VEGFR component is fused to the C-terminus of a second heavy chain of the anti-Ang2 full length antibody via an optional second linker, wherein the third VEGFR component is fused to the C-terminus of a first light chain of the anti-Ang2 full length antibody via an optional third linker, and wherein the fourth VEGFR component is fused to the C-terminus of a second light chain of the anti-Ang2 full length antibody via an optional fourth linker; (3) wherein the first VEGFR component is fused to the N-terminus of a first heavy chain of the anti-Ang2 full length antibody via an optional first linker, wherein the second VEGFR component is fused to the N-terminus of a second heavy chain of the anti-Ang2 full length antibody via an optional second linker, wherein the third VEGFR component is fused to the C-terminus of the first heavy chain of the anti-Ang2 full length antibody via an optional third linker, and wherein the fourth VEGFR component is fused to the C-terminus of the second heavy chain of the anti-Ang2 full length antibody via an optional fourth linker; (4) wherein the first VEGFR component is fused to the C-terminus of a first heavy chain of the anti-Ang2 full length antibody via an optional first linker, wherein the second VEGFR component is fused to the C-terminus of a second heavy chain of the anti-Ang2 full length antibody via an optional second linker, wherein the third VEGFR component is fused to the N-terminus of a first light chain of the anti-Ang2 full length antibody via an optional third linker, and wherein the fourth VEGFR component is fused to the N-terminus of a second light chain of the anti-Ang2 full length antibody via an optional fourth linker; (5) wherein the first VEGFR component is fused to the N-terminus of a first heavy chain of the anti-Ang2 full length antibody via an optional first linker, wherein the second VEGFR component is fused to the N-terminus of a second heavy chain of the anti-Ang2 full length antibody via an optional second linker, wherein the third VEGFR component is fused to the C-terminus of a first light chain of the anti-Ang2 full length antibody via an optional third linker, and wherein the fourth VEGFR component is fused to the C-terminus of a second light chain of the anti-Ang2 full length antibody via an optional fourth linker; or (6) wherein the first VEGFR component is fused to the N-terminus of a first light chain of the anti-Ang2 full length antibody via an optional first linker, wherein the second VEGFR component is fused to the N-terminus of a second light chain of the anti-Ang2 full length antibody via an optional second linker, wherein the third VEGFR component is fused to the C-terminus of the first light chain of the anti-Ang2 full length antibody via an optional third linker, and wherein the fourth VEGFR component is fused to the C-terminus of the second light chain of the anti-Ang2 full length antibody via an optional fourth linker. In some embodiments, the first, second, third, and fourth linkers each comprises an amino acid sequence independently selected from SEQ ID NO: 30 or 31. In some embodiments, the first and second VEGFR components are the same. In some embodiments, the third and fourth VEGFR components are the same. In some embodiments, the four VEGFR components are the same. In some embodiments, at least one VEGFR component is different from the others.

Further provided are isolated nucleic acids encoding any one of the antibody fusion proteins described herein, vectors comprising such nucleic acids, host cells (e.g., CHO cell) comprising such nucleic acids or vectors, and methods of producing any one of the antibody fusion proteins described herein.

Also provided are compositions (e.g., pharmaceutical compositions), kits, and articles of manufacture comprising any of the antibody fusion proteins described herein. Methods of treating a disease or disorder (e.g., cancer, or non-neoplastic disorder such as rheumatoid arthritis, psoriasis, atherosclerosis, hemangiomas, transplant rejection, chronic inflammation, infection, or ocular neovascular disorder (e.g., AMD, diabetic retinopathy)) in an individual using an effective amount of any of the antibody fusion proteins or compositions (e.g., pharmaceutical compositions) described herein are also provided.

### BRIEF DESCRIPTION OF THE DRAWINGS

FIG. 1 shows the effects of exemplary anti-Ang2/VEGF-trap antibody fusion proteins (bispecific antibody fusion proteins, “BsFps”) on Tie2 signaling activation as measured by in vitro phospho-Akt bioassay. Anti-Ang2 antibody hABTAA served as positive control. Empty well (“blank”) and Ang2 protein only served as negative controls.

FIG. 2 shows the effects of exemplary anti-Ang2/VEGF-trap antibody fusion proteins (BsFps) on VEGF neutralization as measured by Bio-Glo™ Luciferase Reporter Assay. Anti-VEGF antibody (Avastin®) served as positive control. Empty well (“blank”) and VEGF protein only (“VEGF165”) served as negative controls.

FIG. 3 shows the effects of exemplary anti-Ang2/VEGF-trap antibody fusion proteins (BsFps) on Tie2 signaling activation as measured by in vitro phospho-Akt bioassay in dose-dependent curves. Anti-Ang2 antibody hABTAA served as positive control.

FIG. 4 shows the effects of exemplary anti-Ang2/VEGF-trap antibody fusion proteins (BsFps) on VEGF neutralization as measured by Bio-Glo™ Luciferase Reporter Assay in dose-dependent curves. Parental VEGF-trap aflibercept (Eylea®) served as positive control.

FIG. 5 depicts a VEGF-trap and an exemplary parental bivalent anti-Ang2 antibody (mAb) for construction of various exemplary anti-Ang2/VEGF-trap antibody fusion proteins.

FIGS. 6A-6J depict exemplary anti-Ang2/VEGF-trap antibody fusion proteins. The VEGF-traps can be the same or different. The parental bivalent anti-Ang2 antibody can be monospecific or bispecific. The VEGF-traps can be fused to polypeptides of the parental bivalent anti-Ang2 antibody either directly or via a linker (e.g., peptide linker).

FIGS. 7A-7C depict models of Ang1/Ang2/Tie2 signaling in the presence or absence of anti-Ang2 antibody, modified based on S. Han et al. (Sci Transl Med. 2016;8(335):335ra55). FIG. 7A depicts no Tie2 signaling when Ang2 competes with Ang1 from binding to Tie2 receptor. FIG. 7B depicts Tie2 receptor clustering and activation upon Ang1 binding, after Ang2 is blocked by anti-Ang2 antagonist antibodies (e.g., ABA). FIG. 7C depicts Ang2 clustering after bound by anti-Ang2 agonist anybody (e.g., ABTAA), and antibody bound-clustered Ang2 triggering Tie2 receptor clustering and activation.

### DETAILED DESCRIPTION OF THE INVENTION

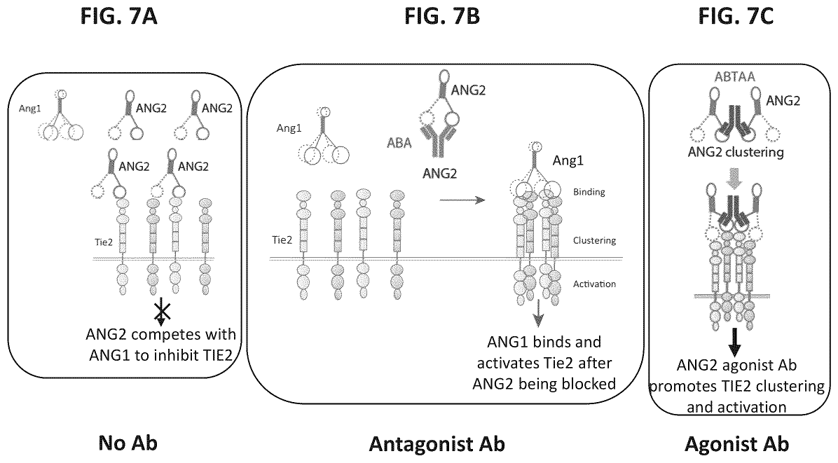

Maintaining endothelial barrier function is critical for vascular normalization, and its dysfunction will lead to several cancers and non-neoplastic diseases. VEGF has been the most widely exploited target for antiangiogenic therapy. However, for treatment of retinal vascular disease, anti-VEGF treatments still do not prevent the progression to legal blindness in many patients. Tie2 is an endothelial cell-specific RTK that is oppositely modulated by Ang1 and Ang2. Ang1 acts as Tie2 agonist leading to its phosphorylation inducing various protective downstream pathways, which induces the stabilization of vascular endothelial cells and reduces vascular permeability by accelerating the junctional integrity of the vascular endothelial cells. Ang2 is an endogenous, context-specific antagonist/agonist of Tie2 by competing with Ang1 as an antagonist (FIG. 7A) or by converting to a potent Tie2 activator. In the absence of Ang1, Ang2 used in high concentration acts as a weak agonist of Tie2. Currently, most Ang2-targeting drugs (e.g., anti-Ang2 antagonist antibody such as ABA) simply block Ang2 binding to Tie2 (FIG. 7B), which eliminate Ang2's important function as a Tie2 agonist. Further, in the context of several diseases or infections like sepsis, Ang2 expression is increased, while Ang1 and Tie2 expression declines, so simply blocking Ang2-Tie2 interaction is unable to maintain vascular normalization. Further, Ang2 promotes the proangiogenic action of VEGF, and VEGF upregulates Ang2 expression in endothelial cells, thus targeting VEGF or Ang2 alone is not sufficient.

The present invention provides antibody fusion proteins comprising: i) a multivalent (e.g., bivalent) anti-Ang2 antibody or antigen-binding fragment thereof, and ii) a VEGFR component, wherein the multivalent anti-Ang2 antibody or antigen-binding fragment thereof does not inhibit the binding between Ang2 and TIE2. These anti-Ang2/VEGF-trap antibody fusion proteins, although fused with a large moiety (the VEGFR component is about 23 kDa), can retain high binding affinity to both VEGF and Ang2 as well as corresponding biological activities (e.g., blocking VEGF/VEGFR signaling, triggering Tie2 signaling activation via Ang2), similarly as the parental VEGF-trap (e.g., aflibercept) and anti-Ang2 agonist antibody (e.g., ABTAA), respectively.

Further, by using an anti-Ang2 agonist antibody or antigen-binding fragment thereof as the antibody fusion protein backbone, which does not inhibit the binding between Ang2 and Tie2 like an anti-Ang2 antagonist antibody, anti-Ang2/VEGF-trap antibody fusion proteins described herein can convert Ang2 to an agonist of Tie2, which is more effective than blocking Ang2-Tie2 interaction in disease treatment, especially in disease conditions where Ang2 expression is increased while Ang1 and Tie2 expression declines. Multivalent (e.g., bivalent) anti-Ang2 agonist antibodies (such as a full length antibody) or antigen-binding fragments thereof used herein as the antibody fusion protein backbone specifically bind to Ang2 and trigger clustering of Ang2, assembling an antibody/Ang2 complex that can subsequently bind, trigger Tie2 clustering, and activate Tie2 (FIG. 7C), resulting in strengthened endothelial barrier, protected endothelial glycocalyx, blunted vascular leakage, and reduced inflammation (S. Han et al., Sci Transl Med. 2016;8(335):335ra55).

Moreover, the dual targeting activity against VEGF and Ang2 of the anti-Ang2/VEGF-trap antibody fusion proteins described herein may increase cellular specificity of the VEGFR component and/or the multivalent anti-Ang2 antibody or antigen-binding fragment thereof by reducing undesired activity in the absence of binding to a target cell of interest (e.g. cancer cells or tissues expressing VEGF, Ang2, and/or Tie2). This feature will be particularly useful to selectively mask or reduce cytotoxicity/over-activation/over-repression of the antibody fusion protein, thereby protecting non-target or normal cells from unwanted or toxic effects while only exposing target cells (e.g. cancer cells or tissues expressing VEGF, Ang2, and/or Tie2) to toxic effects. The antibody fusion protein may also stabilize/enhances VEGF dimer binding by the VEGFR component, and/or stabilize/enhances Ang2 binding by the multivalent anti-Ang2 antibody or antigen-binding fragment thereof, compared to when they are not fused together to form the antibody fusion protein. The dual targeting activity against VEGF and Ang2 of the anti-Ang2/VEGF-trap antibody fusion proteins described herein can provide a synergistic effect that is more effective than targeting either pathway alone, and can overcome drug resistance of single drug (e.g., anti-VEGF antibody or VEGF-trap alone, or anti-Ang2 antibody alone), demonstrating promising therapeutic efficacy.

Also provided are compositions (such as pharmaceutical compositions), kits and articles of manufacture comprising the anti-Ang2/VEGF-trap antibody fusion proteins, methods of making thereof, and methods of treating disease (e.g., cancer, non-neoplastic disorders such as ocular neovascular disorders) using the antibody fusion proteins.

### I. Definitions

As used herein, when a binding domain (e.g., antibody, antigen-binding fragment, or receptor such as VEGFR) is referred to as an “antagonist” of a target molecule (e.g., antigen such as Ang2, or ligand such as VEGF), it means that upon target molecule binding, the binding domain blocks, suppresses, or reduces (e.g., reduces at least about any of 10%, 20%, 30%, 40%, 50%, 60%, 70%, 80%, 90%, or 100%) the biological activity of the target molecule (e.g., blocks receptor signaling). For example, an anti-Ang2 antagonist antibody is an antibody that reduces or blocks Ang2 from binding to its receptor Tie2, such as Ang2-blocking antibody (“ABA”). For example, a VEGF-trap is an antagonist to VEGF, as it blocks or reduces VEGF from binding to and activating VEGFR on cell surface. When a binding domain (e.g., antibody, antigen-binding fragment, or ligand/receptor) is referred to as an “agonist” of a target molecule (e.g., antigen such as Ang2, or receptor/ligand), it means that upon target molecule binding, the binding domain stimulates, activates, or enhances (e.g., enhances at least about any of 10%, 20%, 30%, 40%, 50%, 60%, 70%, 80%, 90%, 100%, or more) the biological activity of the target molecule (e.g., activates receptor signaling). For example, an anti-Ang2 agonist antibody is an antibody that activates or enhances Ang2/Tie2 signaling, such as Ang2-binding and Tie2 agonist antibody (“ABTAA”) which upon binding to Ang2 triggers Ang2 clustering, resulting in clustering and activation of Tie2 receptor.

As used herein, “angiogenesis,” “angiogenic,” or “neovascularization” refers to formation, growth, and/or development of new blood vessels.

The term “neovascular disorder” used herein refers to a disorder characterized by altered or unregulated angiogenesis other than one accompanying oncogenic or neoplastic transformation, i.e., cancer. Examples of neovascular disorders include psoriasis, rheumatoid arthritis, and ocular neovascular disorders such as diabetic retinopathy and age-related macular degeneration (AMD).

The term “ocular neovascular disorder” used herein refers to a disorder characterized by altered or unregulated angiogenesis in the eye of a patient. Exemplary ocular neovascular disorders include optic disc neovascularization, iris neovascularization, retinal neovascularization (RNV), choroidal neovascularization (CNV), corneal neovascularization, vitreal neovascularization, glaucoma, pannus, pterygium, macular edema, diabetic retinopathy, diabetic macular edema, macular degeneration and related conditions (e.g., AMD), vascular retinopathy, retinal degeneration, uveitis, inflammatory diseases of the retina, and proliferative vitreoretinopathy.

“Macular degeneration related condition” refers to any of a number of disorders and conditions in which the macula degenerates or loses functional activity. The degeneration or loss of functional activity can arise as a result of, for example, cell death, decreased cell proliferation, and/or loss of normal biological function. Macular degeneration can lead to and/or manifest as alterations in the structural integrity of the cells and/or extracellular matrix of the macula, alteration in normal cellular and/or extracellular matrix architecture, and/or the loss of function of macular cells. The cells can be any cell type normally present in or near the macula including RPE cells, photoreceptors, and/or capillary endothelial cells. AMD is the major macular degeneration related condition. Others include Best macular dystrophy, Sorsby fundus dystrophy, Mallatia Leventinese and Doyne honeycomb retinal dystrophy.

The terms “angiogenesis inhibitor,” “antiangiogenic agent,” and “antiangiogenic therapy” are used interchangeably herein to refer to agents/therapies that are capable of inhibiting or reducing one or more processes associated with angiogenesis including, but not limited to, endothelial cell proliferation, endothelial cell survival, endothelial cell migration, differentiation of precursor cells into endothelial cells, and capillary tube formation.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the invention contemplate any one or more of these aspects of treatment.

The term “prevent,” and similar words such as “prevented,” “preventing” etc., indicate an approach for preventing, inhibiting, or reducing the likelihood of the recurrence of, a disease or condition, e.g., cancer, neovascular disorder (such as ocular neovascular disorder). It also refers to delaying the recurrence of a disease or condition or delaying the recurrence of the symptoms of a disease or condition. As used herein, “prevention” and similar words also includes reducing the intensity, effect, symptoms and/or burden of a disease or condition prior to recurrence of the disease or condition.

As used herein, “delaying” the development of cancer or neovascular disorder (such as ocular neovascular disorder) means to defer, hinder, slow, retard, stabilize, and/or postpone development of the disease. This delay can be of varying lengths of time, depending on the history of the disease and/or individual being treated. A method that “delays” development of cancer or neovascular disorder (such as ocular neovascular disorder) is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer or neovascular disorder (such as ocular neovascular disorder) progression that may be initially undetectable and includes occurrence, recurrence, and onset.

The term “effective amount” used herein refers to an amount of an agent or a combination of agents, sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the drug or composition may achieve one or more of the following: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) relieve to some extent one or more of the symptoms associated with the cancer; and (viii) stimulate or activate immune cells (e.g., immune effector cells), e.g. for immune response, such as to produce cytokine(s), or for immune cell proliferation and/or differentiation. In reference to ocular neovascular disorder, an effective amount comprises an amount sufficient to treat, suppress, delay and/or prevent a neovascular disorder or symptom thereof, e.g., an amount sufficient to achieve one or more of the following: (i) inhibit or prevent drusen formation; (ii) cause a reduction in drusen number and/or size (drusen regression); (iii) cause a reduction in or prevent lipofuscin deposits; (iv) inhibit or prevent visual loss or slow the rate of visual loss; (v) inhibit choroidal neovascularization or slow the rate of choroidal neovascularization; (vi) cause a reduction in size and/or number of lesions characterized by choroidal neovascularization; (vii) inhibit choroidal neovascularization or slow the rate of retinal neovascularization; (viii) cause a reduction in size and/or number of lesions characterized by retinal neovascularization; (ix) improve visual acuity and/or contrast sensitivity; (x) reduce macular edema and/or reduce abnormal macular thickness; (xi) inhibit or prevent photoreceptor or RPE cell atrophy or apoptosis, or reduce the rate of photoreceptor or RPE cell atrophy or apoptosis; and (xii) inhibit or prevent progression of non-exudative macular degeneration to exudative macular degeneration.

As used herein, an “individual” or a “subject” refers to a mammal, including, but not limited to, human, bovine, horse, feline, canine, rodent, or primate. In some embodiments, the individual is a human.

The term “antibody” is used in its broadest sense and encompasses various antibody structures, including but not limited to monoclonal antibodies, polyclonal antibodies, multi specific antibodies (e.g., bispecific antibodies), full-length antibodies and antigen-binding fragments thereof, so long as they exhibit the desired antigen-binding activity. The term “antibody” includes conventional 4-chain antibodies, single-domain antibodies, and antigen-binding fragments thereof.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen-binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the a and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1 and IgA2.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. Heavy-chain only antibodies from the Camelid species have a single heavy chain variable region, which is referred to as “V_{H}H”. V_{H}H is thus a special type of V_{H}.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called complementary determining regions (CDRs) or hypervariable regions (HVRs) both in the heavy chain and light chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method.

The terms “full-length antibody”, “intact antibody”, or “whole antibody” are used interchangeably to refer to an antibody in its substantially intact form, as opposed to an antibody fragment. Specifically, full-length 4-chain antibodies include those with heavy and light chains including an Fc region. Full-length heavy-chain only antibodies include the heavy chain variable domain (such as V_{H}H) and an Fc region. The constant domains may be native sequence constant domains (e.g., human native sequence constant domains) or amino acid sequence variants thereof. In some cases, the intact antibody may have one or more effector functions.

An “antibody fragment”, “antigen-binding domain”, or “antigen-binding fragment” comprises a portion of an intact antibody, preferably the antigen binding and/or the variable region of the intact antibody. Examples of antibody fragments include, but are not limited to Fab, Fab′, F(ab′)₂ and Fv fragments; diabodies; linear antibodies (see U.S. Pat. No. 5,641,870, Example 2; Zapata et al., Protein Eng. 8(10): 1057-1062 (1995)); single-chain antibody (scFv) molecules; single-domain antibodies (such as V_{H}H), and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy-terminus of the C_{H}1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the CHL (or CL) domain of the light chain.

The “heavy chain” of antibodies (immunoglobulins) can be divided into three functional regions: the Fd region, the hinge region, and the Fc region (fragment crystallizable). The Fd region comprises the V_{H} and C_{H}1 domains and, in combination with the light chain, forms Fab—the antigen-binding fragment. The Fc fragment is responsible for the immunoglobulin effector functions, which include, for example, complement fixation and binding to cognate Fc receptors of effector cells. The hinge region, found in IgG, IgA, and IgD immunoglobulin classes, acts as a flexible spacer that allows the Fab portion to move freely in space relative to the Fc region. In contrast to the constant regions, the hinge domains are structurally diverse, varying in both sequence and length among immunoglobulin classes and subclasses. For heavy-chain only antibody, “heavy chain” includes the heavy chain variable domain (such as V_{H}H), a hinge region, and an Fc region.

The “light chains” of antibodies (immunoglobulins) from any mammalian species can be assigned to one of two clearly distinct types, called kappa (“K”) and lambda (“k”), based on the amino acid sequences of their constant domains.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the scFv polypeptide further comprises a polypeptide linker between the V_{H} and V_{L} domains which enables the scFv to form the desired structure for antigen binding. For a review of the scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. “Humanized antibody” is used as a subset of “chimeric antibodies”.

“Humanized” forms of non-human (e.g., llama or camelid) antibodies are chimeric antibodies that contain minimal sequence derived from non-human immunoglobulin. In some embodiments, a humanized antibody is a human immunoglobulin (recipient antibody) in which residues from an CDR (hereinafter defined) of the recipient are replaced by residues from an CDR of a non-human species (donor antibody) such as mouse, rat, rabbit, camel, llama, alpaca, or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, single-domain antibodies comprise three HVRs (or CDRs): HVR1 (or CDR1), HVR2 (or CDR2), and HVR3 (or CDR3). HVR3 (or CDR3) displays the most diversity of the three HVRs and is believed to play a unique role in conferring fine specificity to antibodies. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below in Table A. HVRs may comprise “extended HVRs” as follows: 24-36 or 24-34 (L1), 46-56 or 50-56 (L2) and 89-97 or 89-96 (L3) in the V L and 26-35 (H1), 50-65 or 49-65 (H2) and 93-102, 94-102, or 95-102 (H3) in the V_{H}. The variable domain residues are numbered according to Kabat et al., supra, for each of these definitions. Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

TABLE A
HVR delineations.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |

Kabat Numbering

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

The term “epitope” means a protein determinant capable of specific binding to a binding domain (e.g., antibody, antigen-binding fragment, or receptor). Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

As used herein, the term “specifically binds”, “specifically recognizes”, or is “specific for” refers to measurable and reproducible interactions such as binding between a target molecule (e.g., ligand) and a binding domain (e.g., antibody, antigen-binding fragment, receptor such as VEGFR component), which is determinative of the presence of the target molecule in the presence of a heterogeneous population of molecules including biological molecules. For example, a binding domain (e.g., antigen-binding fragment or receptor) that specifically binds a target (which can be an epitope) is a binding domain that binds this target with greater affinity, avidity, more readily, and/or with greater duration than it binds other targets. In some embodiments, the extent of binding of a binding domain (e.g., antigen-binding fragment or receptor) to an unrelated target molecule (e.g., unrelated antigen or ligand) is less than about 10% of the binding of the binding domain to the target molecule as measured, e.g., by a radioimmunoassay (MA). In some embodiments, a binding domain (e.g., antibody, antigen-binding fragment, receptor) that specifically binds a target (or a VEGFR component that specifically binds VEGF) has a dissociation constant (K_{D}) of ≤10⁻⁵ M, ≤10⁻⁶ M, ≤10⁻⁷ M, ≤10⁻⁸ M, ≤10⁻⁹ M, ≤10⁻¹⁹ M, ≤10⁻¹¹ M, or ≤10⁻¹² M. In some embodiments, a binding domain specifically binds an epitope on a protein (e.g., antigen, ligand) that is conserved among the protein from different species. In some embodiments, specific binding can include, but does not require exclusive binding. Binding specificity of the binding domain can be determined experimentally by any protein binding methods known in the art. Such methods comprise, but are not limited to Western blots, ELISA-, RIA-, ECL-, IRMA-, EIA-, BIACORE™-tests and peptide scans.

The term “specificity” refers to selective recognition of an antigen binding protein (e.g., antibody, antigen-binding fragment, antibody fusion protein, receptor such as VEGFR component) for a particular epitope of a target molecule. Natural antibodies, for example, are monospecific. The term “multispecific” as used herein denotes that an antigen binding protein (e.g., antibody, antibody fusion protein) has polyepitopic specificity (i.e., is capable of specifically binding to two, three, or more, different epitopes on one biological molecule or is capable of specifically binding to epitopes on two, three, or more, different biological molecules). “Bispecific” as used herein denotes that an antigen binding protein has two different antigen-binding specificities. Unless otherwise indicated, the order in which the antigens bound by a bispecific antibody or antibody fusion protein listed is arbitrary. That is, for example, the terms “anti-Ang2/VEGF-trap,” and “VEGF-trap/anti-Ang2” may be used interchangeably to refer to bispecific antibody fusion protein that specifically bind to both Ang2 and VEGF. The term “monospecific” as used herein denotes an antigen binding protein that has one or more binding sites each of which bind the same epitope of the same antigen.

The term “valent” as used herein denotes the presence of a specified number of binding sites in an antigen binding protein. A natural antibody for example or a full-length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein. “Multivalent” can be bivalent, trivalent, tetravalent, or more.

“Antibody effector functions” refer to those biological activities attributable to the Fc region (a native sequence Fc region or amino acid sequence variant Fc region) of an antibody and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptors); and B cell activation. “Reduced or minimized” antibody effector function means that which is reduced by at least 50% (alternatively 60%, 65%, 70%, 75%, 80%, 85%, 90%, 95%, 96%, 97%, 98%, or 99%) from the wild type or unmodified antibody. The determination of antibody effector function is readily determinable and measurable by one of ordinary skill in the art.

“Antibody-dependent cell-mediated cytotoxicity” or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. Antibody variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

The term “Fc region,” “fragment crystallizable region,” “Fc fragment,” or “Fc domain” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody or Fc-fusion protein, or by recombinantly engineering the nucleic acid encoding a heavy chain of the antibody or Fc-fusion protein. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use include but are not limited to human IgG1, IgG2 (IgG2A, IgG2B), IgG3 and IgG4.

The term IgG “isotype” or “subclass” as used herein is meant any of the subclasses of immunoglobulins defined by the chemical and antigenic characteristics of their constant regions. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000).

“Fc receptor” or “FcR” describes a receptor that binds the Fc region of an antibody or Fc-fusion protein. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9(2): 6591-6604 (2001).

“Binding affinity” generally refers to the strength of the sum total of non-covalent interactions between a single binding site of a molecule (e.g., an antibody, antigen-binding fragment, VEGFR) and its binding partner (e.g., an antigen, VEGF). Unless indicated otherwise, as used herein, “binding affinity” refers to intrinsic binding affinity that reflects a 1:1 interaction between members of a binding pair. Binding affinity can be indicated by K_{d}, K_{off}, Kₒₙ, or Kₐ. The term “K_{off}”, as used herein, is intended to refer to the off rate constant for dissociation of a molecule from its binding partner, as determined from a kinetic selection set up, expressed in units of s⁻¹. The term “Kₒₙ”, as used herein, is intended to refer to the on rate constant for association of a molecule to its binding partner to form the molecule/binding partner complex, expressed in units of M⁻¹ s⁻¹. The term equilibrium dissociation constant “K_{D}” or “K_{d}”, as used herein, refers to the dissociation constant of a particular molecule-binding partner interaction, and describes the concentration of molecule required to occupy one half of all of the binding partner present in a solution of binding partner at equilibrium, and is equal to K_{off}/Kₒₙ, expressed in units of M. The measurement of K_{d} presupposes that all binding agents are in solution. In the case where the antibody is tethered to a cell wall, e.g., in a yeast expression system, the corresponding equilibrium rate constant is expressed as EC50, which gives a good approximation of K_{d}. The affinity constant, Kₐ, is the inverse of the dissociation constant, K_{d}, expressed in units of M⁻¹. The dissociation constant (K_{D} or K_{d}) is used as an indicator showing affinity of binding partners to molecules. For example, easy analysis is possible by the Scatchard method using antibodies marked with a variety of marker agents, as well as by using BiacoreX (made by Amersham Biosciences), which is an over-the-counter, measuring kit, or similar kit, according to the user's manual and experiment operation method attached with the kit. The K_{D} value that can be derived using these methods is expressed in units of M (Mols). An antibody or antigen-binding fragment thereof that specifically binds to a target (or a VEGFR component described herein that specifically binds to VEGF) may have a dissociation constant (K d) of, for example, ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M.

Half maximal inhibitory concentration (IC₅₀) is a measure of the effectiveness of a substance (such as an antibody, antigen-binding fragment, or VEGFR) in inhibiting a specific biological or biochemical function. It indicates how much of a particular drug or other substance (inhibitor, such as an antibody, antigen-binding fragment, or VEGFR) is needed to inhibit a given biological process, or component of a process, i.e. an enzyme, cell, cell receptor or microorganism) by half. The values are typically expressed as molar concentration. IC₅₀ is comparable to an EC₅₀ for agonist drug or other substance (such as an antibody, antigen-binding fragment). EC₅₀ also represents the plasma concentration required for obtaining 50% of a maximum effect in vivo. As used herein, an “IC₅₀” is used to indicate the effective concentration of an antibody or antigen-binding fragment needed to neutralize/reduce 50% of the antigen bioactivity (or the effective concentration of VEGFR component needed to neutralize/reduce 50% of the VEGF bioactivity) in vitro. IC₅₀ or EC₅₀ can be measured by bioassays such as inhibition of ligand binding by FACS analysis (competition binding assay), cell based cytokine release assay, cell-signaling assay (e.g., Phospho-Akt), luciferase reporter gene expression assay, or amplified luminescent proximity homogeneous assay (AlphaLISA). Also see Example 1 for exemplary methods.

“Covalent bond” as used herein refers to a stable bond between two atoms sharing one or more electrons. Examples of covalent bonds include, but are not limited to, peptide bonds and disulfide bonds. As used herein, “peptide bond” refers to a covalent bond formed between a carboxyl group of an amino acid and an amine group of an adjacent amino acid. A “disulfide bond” as used herein refers to a covalent bond formed between two sulfur atoms, such as a combination of two Fc fragments by one or more disulfide bonds. One or more disulfide bonds may be formed between the two fragments by linking the thiol groups in the two fragments. In some embodiments, one or more disulfide bonds can be formed between one or more cysteines of two Fc fragments. Disulfide bonds can be formed by oxidation of two thiol groups.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full-length of the sequences being compared.

As used herein, the “C terminus” of a polypeptide refers to the last amino acid residue of the polypeptide which donates its amine group to form a peptide bond with the carboxyl group of its adjacent amino acid residue. “N terminus” of a polypeptide as used herein refers to the first amino acid of the polypeptide which donates its carboxyl group to form a peptide bond with the amine group of its adjacent amino acid residue.

An “isolated” nucleic acid molecule encoding a construct is a nucleic acid molecule that is identified and separated from at least one contaminant nucleic acid molecule with which it is ordinarily associated in the environment in which it was produced. The isolated nucleic acid molecules encoding the constructs is in a form other than in the form or setting in which it is found in nature, also including nucleic acid molecules present extrachromosomally or at a chromosomal location that is different from its natural chromosomal location.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

“Biocompatible” refers to a material that is substantially nontoxic to a recipient's cells in the quantities and at the location used, and does not elicit or cause a significant deleterious or untoward effect on the recipient's body at the location used, e.g., an unacceptable immunological or inflammatory reaction, unacceptable scar tissue formation, etc. For example, a material that is biocompatible with the eye does not substantially interfere with the physiology or function of the eye.

“Bioerodible” or “biodegradable” means that a material is capable of being broken down physically and/or chemically within cells or within the body of a subject, e.g., by hydrolysis under physiological conditions and/or by natural biological processes such as the action of enzymes present within cells or within the body, and/or by processes such as dissolution, dispersion, etc., to form smaller chemical species which can typically be metabolized and, optionally, used by the body, and/or excreted or otherwise disposed of. Preferably a biodegradable compound is biocompatible. A polymer whose molecular weight decreases over time in vivo due to a reduction in the number of monomers is considered biodegradable.

The term “pharmaceutical formulation” of “pharmaceutical composition” refers to a preparation that is in such form as to permit the biological activity of the active ingredient to be effective, and that contains no additional components that are unacceptably toxic to a subject to which the formulation would be administered. Such formulations are sterile. A “sterile” formulation is aseptic or free from all living microorganisms and their spores.

It is understood that embodiments of the invention described herein (such as “comprising” embodiments) include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### II. Antibody Fusion Proteins Comprising a Multivalent Anti-Ang2 Antibody or Antigen-Binding Fragment Thereof and a VEGFR Component

The present invention in one aspect provides an antibody fusion protein (“anti-Ang2/VEGF-trap antibody fusion protein”) comprising: i) a multivalent (e.g., bivalent) anti-Ang2 antibody (e.g., full length antibody) or antigen-binding fragment thereof, and ii) a VEGFR component, wherein the multivalent anti-Ang2 antibody or antigen-binding fragment thereof does not inhibit the binding between Ang2 and Tie2. In some embodiments, there is provided an antibody fusion protein comprising: i) an anti-Ang2 full length antibody, and ii) a VEGFR component, wherein the anti-Ang2 full length antibody does not inhibit the binding between Ang2 and Tie2. In some embodiments, the multivalent anti-Ang2 antibody or antigen-binding fragment thereof upon binding to Ang2 activates Tie2 signaling through the antibody-bound Ang2.

In some embodiments, the VEGFR component is fused to the multivalent (e.g., bivalent) anti-Ang2 antibody (e.g., full length antibody) or antigen-binding fragment thereof via an optional linker (e.g., SEQ ID NO: 30 or 31). In some embodiments, the anti-Ang2/VEGF-trap antibody fusion protein comprises only one VEGFR component. In some embodiments, the anti-Ang2/VEGF-trap antibody fusion protein comprises two or more (e.g., 2, 3, 4, or more) VEGFR components. In some embodiments, two or more VEGFR components are fused in tandem via one or more optional linkers (can be the same (e.g., SEQ ID NO: 30 or 31) or different from the linker that connects VEGFR component and the multivalent anti-Ang2 antibody or antigen-binding fragment thereof), which is then fused to the multivalent anti-Ang2 antibody or antigen-binding fragment thereof via an optional linker (e.g., SEQ ID NO: 30 or 31). In some embodiments, each anti-Ang2 binding domain of the multivalent anti-Ang2 antibody or antigen-binding fragment thereof is fused with a VEGFR component via an optional linker. In some embodiments, not all anti-Ang2 binding domains of the multivalent anti-Ang2 antibody or antigen-binding fragment thereof is fused with a VEGFR component. In some embodiments, the VEGFR component is fused to a connecting moiety (e.g., Fc fragment or portion thereof such as CH2 only or portion thereof, CH3 only or portion thereof, or CH2-CH3 or portion thereof, or albumin protein) of the multivalent anti-Ang2 antibody or antigen-binding fragment thereof that connects two or more anti-Ang2 binding domains. In some embodiments, the two or more VEGFR components fused to the multivalent anti-Ang2 antibody or antigen-binding fragment thereof are the same. In some embodiments, the two or more VEGFR components fused to the multivalent anti-Ang2 antibody or antigen-binding fragment thereof are different from each other, such as comprising different Ig-like domains, different numbers of Ig-like domains, and/or different organizations (N′ to C′ arrangement) of the Ig-like domains. In some embodiments, at least one VEGFR component is different from the other VEGFR components fused to the multivalent anti-Ang2 antibody or antigen-binding fragment thereof. In some embodiments, each VEGFR component is fused to the multivalent anti-Ang2 antibody or antigen-binding fragment thereof via a linker (e.g., SEQ ID NO: 30 or 31). In some embodiments, not all VEGFR components are fused to the multivalent anti-Ang2 antibody or antigen-binding fragment thereof via linkers (such as fused directly). In some embodiments, each VEGFR component is fused to the multivalent anti-Ang2 antibody or antigen-binding fragment thereof directly without a linker. In some embodiments, all linkers are the same. In some embodiments, at least one linker is different from the other linker(s). In some embodiments, all linkers are different, such as comprising different linker type (e.g., peptide linker vs. chemical linker), different sequences, and/or different length.

In some embodiments, the antibody fusion protein comprises only one VEGFR component, and binding to VEGF dimer via the VEGFR component brings two antibody fusion proteins in proximity, i.e., each antibody fusion protein contributes a VEGFR component to form a VEGF-trap that binds the VEGF dimer. In some embodiments, such design enables further clustering of Ang2 to bind to anti-Ang2 binding domains of the “anti-Ang2/VEGFR[VEGF dimer]VEGFR/anti-Ang2” complex (e.g., compared to the clustering of Ang2 by an anti-Ang2/VEGF-trap antibody fusion protein comprising two or more VEGFR components), leading to one or more of i) stronger binding affinity of antibody-bound Ang2 to Tie2, ii) further clustering of Tie2, or iii) stronger activation of Tie2.

In some embodiments, the multivalent anti-Ang2 antibody or antigen-binding fragment thereof is a full length antibody (“anti-Ang2 full length antibody,” or “bivalent anti-Ang2 full length antibody”). In some embodiments, the anti-Ang2 full length antibody is monospecific. In some embodiments, the anti-Ang2 full length antibody is bispecific.

In some embodiments, there is provided an antibody fusion protein comprising i) an anti-Ang2 full length antibody, and ii) a VEGFR component (e.g., SEQ ID NO: 32), wherein the anti-Ang2 full length antibody does not inhibit the binding between Ang2 and Tie2, and wherein the anti-Ang2 full length antibody comprises any of the following: (1) i) an HC-CDR1 comprising the amino acid sequence of SEQ ID NO: 1; ii) an HC-CDR2 comprising the amino acid sequence of SEQ ID NO: 7; iii) an HC-CDR3 comprising the amino acid sequence of SEQ ID NO: 12; iv) an LC-CDR1 comprising the amino acid sequence of SEQ ID NO: 17; v) an LC-CDR2 comprising the amino acid sequence of SEQ ID NO: 19; and vi) an LC-CDR3 comprising the amino acid sequence of SEQ ID NO: 24; (2) i) an HC-CDR1 comprising the amino acid sequence of SEQ ID NO: 2; ii) an HC-CDR2 comprising the amino acid sequence of SEQ ID NO: 8; iii) an HC-CDR3 comprising the amino acid sequence of SEQ ID NO: 13; iv) an LC-CDR1 comprising the amino acid sequence of SEQ ID NO: 17; v) an LC-CDR2 comprising the amino acid sequence of SEQ ID NO: 20; and vi) an LC-CDR3 comprising the amino acid sequence of SEQ ID NO: 25; (3) i) an HC-CDR1 comprising the amino acid sequence of SEQ ID NO: 3; ii) an HC-CDR2 comprising the amino acid sequence of SEQ ID NO: 9; iii) an HC-CDR3 comprising the amino acid sequence of SEQ ID NO: 14; iv) an LC-CDR1 comprising the amino acid sequence of SEQ ID NO: 17; v) an LC-CDR2 comprising the amino acid sequence of SEQ ID NO: 21; and vi) an LC-CDR3 comprising the amino acid sequence of SEQ ID NO: 26; (4) i) an HC-CDR1 comprising the amino acid sequence of SEQ ID NO: 4; ii) an HC-CDR2 comprising the amino acid sequence of SEQ ID NO: 7; iii) an HC-CDR3 comprising the amino acid sequence of SEQ ID NO: 12; iv) an LC-CDR1 comprising the amino acid sequence of SEQ ID NO: 17; v) an LC-CDR2 comprising the amino acid sequence of SEQ ID NO: 19; and vi) an LC-CDR3 comprising the amino acid sequence of SEQ ID NO: 24; (5) i) an HC-CDR1 comprising the amino acid sequence of SEQ ID NO: 5; ii) an HC-CDR2 comprising the amino acid sequence of SEQ ID NO: 10; iii) an HC-CDR3 comprising the amino acid sequence of SEQ ID NO: 15; iv) an LC-CDR1 comprising the amino acid sequence of SEQ ID NO: 18; v) an LC-CDR2 comprising the amino acid sequence of SEQ ID NO: 22; and vi) an LC-CDR3 comprising the amino acid sequence of SEQ ID NO: 27; or (6) i) an HC-CDR1 comprising the amino acid sequence of SEQ ID NO: 6; ii) an HC-CDR2 comprising the amino acid sequence of SEQ ID NO: 11; iii) an HC-CDR3 comprising the amino acid sequence of SEQ ID NO: 16; iv) an LC-CDR1 comprising the amino acid sequence of SEQ ID NO: 17; v) an LC-CDR2 comprising the amino acid sequence of SEQ ID NO: 23; and vi) an LC-CDR3 comprising the amino acid sequence of SEQ ID NO: 28. In some embodiments, there is provided an antibody fusion protein comprising i) an anti-Ang2 full length antibody, and ii) a VEGFR component (e.g., SEQ ID NO: 32), wherein the anti-Ang2 full length antibody does not inhibit the binding between Ang2 and Tie2, and wherein the anti-Ang2 full length antibody comprises any of the following: (1) a VH comprising the amino acid sequence of SEQ ID NO: 82 or 83, and an VL comprising the amino acid sequence of any of SEQ ID NOs: 84-86; (2) a VH comprising the amino acid sequence of SEQ ID NO: 87, and an VL comprising the amino acid sequence of SEQ ID NO: 88 or 98; (3) a VH comprising the amino acid sequence of SEQ ID NO: 89 or 90, and an VL comprising the amino acid sequence of SEQ ID NO: 91 or 92; (4) a VH comprising the amino acid sequence of SEQ ID NO: 93, and an VL comprising the amino acid sequence of any of SEQ ID NOs: 84-86; (5) a VH comprising the amino acid sequence of SEQ ID NO: 94, and an VL comprising the amino acid sequence of SEQ ID NO: 95; or (6) a VH comprising the amino acid sequence of SEQ ID NO: 96, and an VL comprising the amino acid sequence of SEQ ID NO: 97. In some embodiments, there is provided an antibody fusion protein comprising i) an anti-Ang2 full length antibody, and ii) a VEGFR component (e.g., SEQ ID NO: 32), wherein the anti-Ang2 full length antibody does not inhibit the binding between Ang2 and Tie2, and wherein the anti-Ang2 full length antibody comprises any of the following: (1) a heavy chain comprising the amino acid sequence of SEQ ID NO: 33 or 34, and a light chain comprising the amino acid sequence of any of SEQ ID NOs: 41-43; (2) a heavy chain comprising the amino acid sequence of SEQ ID NO: 35, and a light chain comprising the amino acid sequence of SEQ ID NO: 44 or 49; (3) a heavy chain comprising the amino acid sequence of SEQ ID NO: 36 or 37, and a light chain comprising the amino acid sequence of SEQ ID NO: 45 or 46; (4) a heavy chain comprising the amino acid sequence of SEQ ID NO: 38, and a light chain comprising the amino acid sequence of any of SEQ ID NOs: 41-43; (5) a heavy chain comprising the amino acid sequence of SEQ ID NO: 39, and a light chain comprising the amino acid sequence of SEQ ID NO: 47; or (6) a heavy chain comprising the amino acid sequence of SEQ ID NO: 40, and a light chain comprising the amino acid sequence of SEQ ID NO: 48. In some embodiments, the anti-Ang2 full length antibody upon binding to Ang2 activates Tie2 signaling through the antibody-bound Ang2. In some embodiments, the anti-Ang2 full length antibody is monospecific. In some embodiments, the anti-Ang2 full length antibody is bispecific. In some embodiments, the VEGFR component is fused to the N-terminus of a heavy chain of the anti-Ang2 full length antibody via an optional linker (e.g., SEQ ID NO: 30 or 31). In some embodiments, the VEGFR component is fused to the C-terminus of a heavy chain of the anti-Ang2 full length antibody via an optional linker (e.g., SEQ ID NO: 30 or 31). In some embodiments, the VEGFR component is fused to the N-terminus of a light chain of the anti-Ang2 full length antibody via an optional linker (e.g., SEQ ID NO: 30 or 31). In some embodiments, the VEGFR component is fused to the C-terminus of a light chain of the anti-Ang2 full length antibody via an optional linker (e.g., SEQ ID NO: 30 or 31). In some embodiments, the antibody fusion protein comprises two or more (e.g., 2, 3, 4, 5, 6, 7, 8, or more) VEGFR components. In some embodiments, the two or more VEGFR components is fused to one or more of: i) the N-terminus of one or both heavy chains of the anti-Ang2 full length antibody; ii) the C-terminus of one or both heavy chains of the anti-Ang2 full length antibody; iii) the N-terminus of one or both light chains of the anti-Ang2 full length antibody; or iv) the C-terminus of one or both light chains of the anti-Ang2 full length antibody, via one or more optional linkers (e.g., SEQ ID NO: 30 or 31). In some embodiments, two or more VEGFR components are fused in tandem via one or more optional linkers (can be the same (e.g., SEQ ID NO: 30 or 31) or different from the linker that connects VEGFR component and the anti-Ang2 full length antibody), which is then fused to the anti-Ang2 full length antibody via an optional linker (e.g., SEQ ID NO: 30 or 31). In some embodiments, all linkers are the same. In some embodiments, at least one linker is different from the other linkers. In some embodiments, all linkers are different from each other. In some embodiments, the linkers used for heavy chain fusion of the VEGFR components are the same. In some embodiments, the linkers used for light chain fusion of the VEGFR components are the same. In some embodiments, all VEGFR components are the same. In some embodiments, at least one VEGFR component is different from the other VEGFR components. In some embodiments, all VEGFR components are different from each other.

In some embodiments, there is provided an antibody fusion protein comprising: i) an anti-Ang2 full length antibody, and ii) a VEGFR component (e.g., SEQ ID NO: 32), wherein the anti-Ang2 full length antibody does not inhibit the binding between Ang2 and Tie2, and wherein the VEGFR component is fused to the N-terminus of a heavy chain of the anti-Ang2 full length antibody via an optional linker (e.g., SEQ ID NO: 30 or 31). In some embodiments, the heavy chain fusion polypeptide comprises the amino acid sequence of any of SEQ ID NOs: 56-61, 75, and 79. In some embodiments, there is provided an antibody fusion protein comprising: i) an anti-Ang2 full length antibody, and ii) a VEGFR component, wherein the anti-Ang2 full length antibody does not inhibit the binding between Ang2 and Tie2, wherein the VEGFR component is fused to the N-terminus of a heavy chain of the anti-Ang2 full length antibody via a linker, wherein the antibody fusion protein comprises any of the following: (1) a heavy chain fusion polypeptide comprising the amino acid sequence of SEQ ID NO: 56 or 58, and a light chain comprising the amino acid sequence of SEQ ID NO: 42; (2) a heavy chain fusion polypeptide comprising the amino acid sequence of SEQ ID NO: 57, and a light chain comprising the amino acid sequence of SEQ ID NO: 41 or 42; (3) a heavy chain fusion polypeptide comprising the amino acid sequence of SEQ ID NO: 59, and a light chain comprising the amino acid sequence of SEQ ID NO: 45; (4) a heavy chain fusion polypeptide comprising the amino acid sequence of SEQ ID NO: 60, and a light chain comprising the amino acid sequence of SEQ ID NO: 46; (5) a heavy chain fusion polypeptide comprising the amino acid sequence of SEQ ID NO: 61, and a light chain comprising the amino acid sequence of SEQ ID NO: 44; (6) a heavy chain fusion polypeptide comprising the amino acid sequence of SEQ ID NO: 75, and a light chain comprising the amino acid sequence of SEQ ID NO: 47; or (7) a heavy chain fusion polypeptide comprising the amino acid sequence of SEQ ID NO: 79, and a light chain comprising the amino acid sequence of SEQ ID NO: 48. In some embodiments, there is provided an antibody fusion protein comprising: i) an anti-Ang2 full length antibody, and ii) a VEGFR component, wherein the anti-Ang2 full length antibody does not inhibit the binding between Ang2 and Tie2, wherein the VEGFR component is fused to the N-terminus of a heavy chain of the anti-Ang2 full length antibody via a linker, wherein the antibody fusion protein comprises any of the following: (1) a heavy chain fusion polypeptide comprising the amino acid sequence of SEQ ID NO: 56, a heavy chain comprising the amino acid sequence of SEQ ID NO: 33, and two light chains each comprising the amino acid sequence of SEQ ID NO: 42; (2) a heavy chain fusion polypeptide comprising the amino acid sequence of SEQ ID NO: 58, a heavy chain comprising the amino acid sequence of SEQ ID NO: 38, and two light chains each comprising the amino acid sequence of SEQ ID NO: 42; (3) a heavy chain fusion polypeptide comprising the amino acid sequence of SEQ ID NO: 57, a heavy chain comprising the amino acid sequence of SEQ ID NO: 34, and two light chains each comprising the amino acid sequence of SEQ ID NO: 41; (4) a heavy chain fusion polypeptide comprising the amino acid sequence of SEQ ID NO: 57, a heavy chain comprising the amino acid sequence of SEQ ID NO: 34, and two light chains each comprising the amino acid sequence of SEQ ID NO: 42; (5) a heavy chain fusion polypeptide comprising the amino acid sequence of SEQ ID NO: 59, a heavy chain comprising the amino acid sequence of SEQ ID NO: 37, and two light chains each comprising the amino acid sequence of SEQ ID NO: 45; (6) a heavy chain fusion polypeptide comprising the amino acid sequence of SEQ ID NO: 60, a heavy chain comprising the amino acid sequence of SEQ ID NO: 36, and two light chains each comprising the amino acid sequence of SEQ ID NO: 46; (7) a heavy chain fusion polypeptide comprising the amino acid sequence of SEQ ID NO: 61, a heavy chain comprising the amino acid sequence of SEQ ID NO: 35, and two light chains each comprising the amino acid sequence of SEQ ID NO: 44; (8) a heavy chain fusion polypeptide comprising the amino acid sequence of SEQ ID NO: 75, a heavy chain comprising the amino acid sequence of SEQ ID NO: 39, and two light chains each comprising the amino acid sequence of SEQ ID NO: 47; or (9) a heavy chain fusion polypeptide comprising the amino acid sequence of SEQ ID NO: 79, a heavy chain comprising the amino acid sequence of SEQ ID NO: 40, and two light chains each comprising the amino acid sequence of SEQ ID NO: 48. In some embodiments, the anti-Ang2 full length antibody upon binding to Ang2 activates Tie2 signaling through the antibody-bound Ang2.

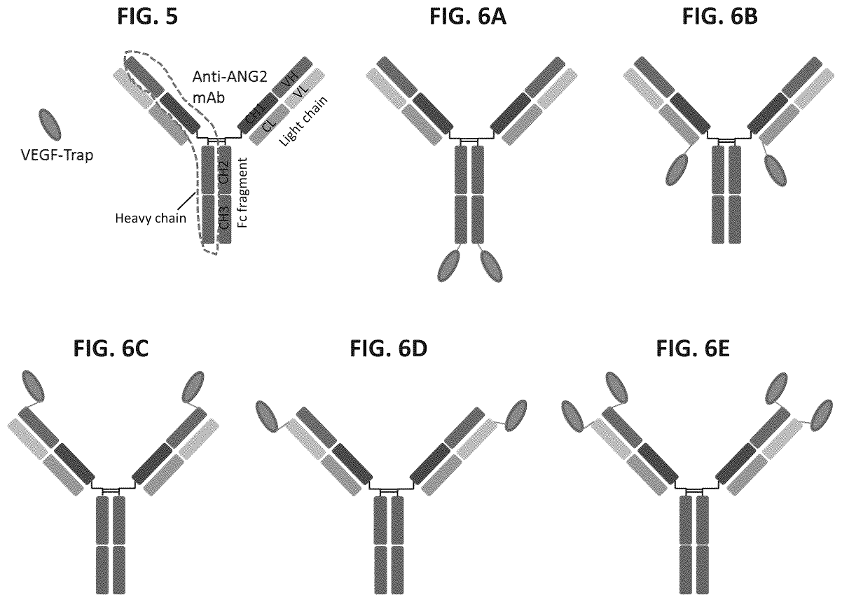

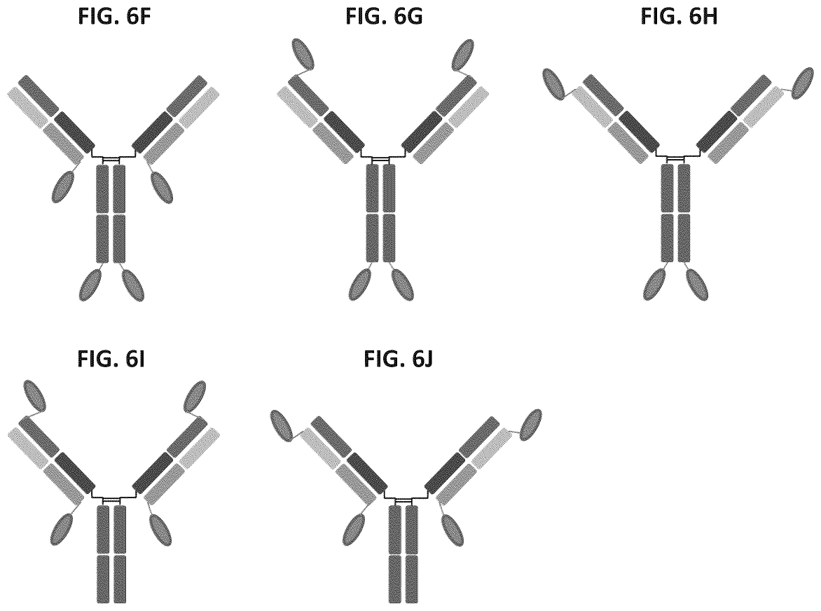

In some embodiments, there is provided an antibody fusion protein comprising: i) an anti-Ang2 full length antibody, ii) a first VEGFR component (e.g., SEQ ID NO: 32), and iii) a second VEGFR component (e.g., SEQ ID NO: 32), wherein the anti-Ang2 full length antibody does not inhibit the binding between Ang2 and Tie2, wherein the first VEGFR component is fused to the N-terminus of a first heavy chain of the anti-Ang2 full length antibody via an optional first linker (e.g., SEQ ID NO: 30 or 31), and wherein the second VEGFR component is fused to the N-terminus of a second heavy chain of the anti-Ang2 full length antibody via an optional second linker (e.g., SEQ ID NO: 30 or 31). See, e.g., FIG. 6C. In some embodiments, the first and second linkers are the same. In some embodiments, the first and second linkers are different. In some embodiments, the first and second VEGFR components are the same. In some embodiments, the first and second VEGFR components are different. In some embodiments, the two heavy chain fusion polypeptides are the same. In some embodiments, there is provided an antibody fusion protein comprising: i) an anti-Ang2 full length antibody, ii) a first VEGFR component, and iii) a second VEGFR component, wherein the anti-Ang2 full length antibody does not inhibit the binding between Ang2 and Tie2, wherein the first VEGFR component is fused to the N-terminus of a first heavy chain of the anti-Ang2 full length antibody via a first linker, wherein the second VEGFR component is fused to the N-terminus of a second heavy chain of the anti-Ang2 full length antibody via a second linker, and wherein the antibody fusion protein comprises any of the following: (1) two heavy chain fusion polypeptides each comprising the amino acid sequence of SEQ ID NO: 56 or 58, and two light chains each comprising the amino acid sequence of SEQ ID NO: 42; (2) two heavy chain fusion polypeptides each comprising the amino acid sequence of SEQ ID NO: 57, and two light chains each comprising the amino acid sequence of SEQ ID NO: 41 or 42; (3) two heavy chain fusion polypeptides each comprising the amino acid sequence of SEQ ID NO: 59, and two light chains each comprising the amino acid sequence of SEQ ID NO: 45; (4) two heavy chain fusion polypeptides each comprising the amino acid sequence of SEQ ID NO: 60, and two light chains each comprising the amino acid sequence of SEQ ID NO: 46; (5) two heavy chain fusion polypeptides each comprising the amino acid sequence of SEQ ID NO: 61, and two light chains each comprising the amino acid sequence of SEQ ID NO: 44; (6) two heavy chain fusion polypeptides each comprising the amino acid sequence of SEQ ID NO: 75, and two light chains each comprising the amino acid sequence of SEQ ID NO: 47; or (7) two heavy chain fusion polypeptides each comprising the amino acid sequence of SEQ ID NO: 79, and two light chains each comprising the amino acid sequence of SEQ ID NO: 48. In some embodiments, the anti-Ang2 full length antibody upon binding to Ang2 activates Tie2 signaling through the antibody-bound Ang2.

In some embodiments, there is provided an antibody fusion protein comprising: i) an anti-Ang2 full length antibody, and ii) a VEGFR component (e.g., SEQ ID NO: 32), wherein the anti-Ang2 full length antibody does not inhibit the binding between Ang2 and Tie2, and wherein the VEGFR component is fused to the C-terminus of a heavy chain of the anti-Ang2 full length antibody via an optional linker (e.g., SEQ ID NO: 30 or 31). In some embodiments, the heavy chain fusion polypeptide comprises the amino acid sequence of any of SEQ ID NOs: 50-55, 74, and 78. In some embodiments, there is provided an antibody fusion protein comprising: i) an anti-Ang2 full length antibody, and ii) a VEGFR component, wherein the anti-Ang2 full length antibody does not inhibit the binding between Ang2 and Tie2, wherein the VEGFR component is fused to the C-terminus of a heavy chain of the anti-Ang2 full length antibody via a linker, and wherein the antibody fusion protein comprises any of the following: (1) a heavy chain fusion polypeptide comprising the amino acid sequence of SEQ ID NO: 50 or 52, and a light chain comprising the amino acid sequence of SEQ ID NO: 42; (2) a heavy chain fusion polypeptide comprising the amino acid sequence of SEQ ID NO: 51, and a light chain comprising the amino acid sequence of SEQ ID NO: 41 or 42; (3) a heavy chain fusion polypeptide comprising the amino acid sequence of SEQ ID NO: 59, and a light chain comprising the amino acid sequence of SEQ ID NO: 45; (4) a heavy chain fusion polypeptide comprising the amino acid sequence of SEQ ID NO: 60, and a light chain comprising the amino acid sequence of SEQ ID NO: 46; (5) a heavy chain fusion polypeptide comprising the amino acid sequence of SEQ ID NO: 61, and a light chain comprising the amino acid sequence of SEQ ID NO: 44; (6) a heavy chain fusion polypeptide comprising the amino acid sequence of SEQ ID NO: 75, and a light chain comprising the amino acid sequence of SEQ ID NO: 47; or (7) a heavy chain fusion polypeptide comprising the amino acid sequence of SEQ ID NO: 79, and a light chain comprising the amino acid sequence of SEQ ID NO: 48. In some embodiments, there is provided an antibody fusion protein comprising: i) an anti-Ang2 full length antibody, and ii) a VEGFR component, wherein the anti-Ang2 full length antibody does not inhibit the binding between Ang2 and Tie2, wherein the VEGFR component is fused to the C-terminus of a heavy chain of the anti-Ang2 full length antibody via a linker, and wherein the antibody fusion protein comprises any of the following: (1) a heavy chain fusion polypeptide comprising the amino acid sequence of SEQ ID NO: 50, a heavy chain comprising the amino acid sequence of SEQ ID NO: 33, and two light chains each comprising the amino acid sequence of SEQ ID NO: 42; (2) a heavy chain fusion polypeptide comprising the amino acid sequence of SEQ ID NO: 52, a heavy chain comprising the amino acid sequence of SEQ ID NO: 38, and two light chains each comprising the amino acid sequence of SEQ ID NO: 42; (3) a heavy chain fusion polypeptide comprising the amino acid sequence of SEQ ID NO: 51, a heavy chain comprising the amino acid sequence of SEQ ID NO: 34, and two light chains each comprising the amino acid sequence of SEQ ID NO: 41; (4) a heavy chain fusion polypeptide comprising the amino acid sequence of SEQ ID NO: 51, a heavy chain comprising the amino acid sequence of SEQ ID NO: 34, and two light chains each comprising the amino acid sequence of SEQ ID NO: 42; (5) a heavy chain fusion polypeptide comprising the amino acid sequence of SEQ ID NO: 59, a heavy chain comprising the amino acid sequence of SEQ ID NO: 37, and two light chains each comprising the amino acid sequence of SEQ ID NO: 45; (6) a heavy chain fusion polypeptide comprising the amino acid sequence of SEQ ID NO: 60, a heavy chain comprising the amino acid sequence of SEQ ID NO: 36, and two light chains each comprising the amino acid sequence of SEQ ID NO: 46; (7) a heavy chain fusion polypeptide comprising the amino acid sequence of SEQ ID NO: 61, a heavy chain comprising the amino acid sequence of SEQ ID NO: 35, and two light chains each comprising the amino acid sequence of SEQ ID NO: 44; (8) a heavy chain fusion polypeptide comprising the amino acid sequence of SEQ ID NO: 75, a heavy chain comprising the amino acid sequence of SEQ ID NO: 39, and two light chains each comprising the amino acid sequence of SEQ ID NO: 47; or (9) a heavy chain fusion polypeptide comprising the amino acid sequence of SEQ ID NO: 79, a heavy chain comprising the amino acid sequence of SEQ ID NO: 40, and two light chains each comprising the amino acid sequence of SEQ ID NO: 48. In some embodiments, the anti-Ang2 full length antibody upon binding to Ang2 activates Tie2 signaling through the antibody-bound Ang2.

In some embodiments, there is provided an antibody fusion protein comprising: i) an anti-Ang2 full length antibody, ii) a first VEGFR component (e.g., SEQ ID NO: 32), and iii) a second VEGFR component (e.g., SEQ ID NO: 32), wherein the anti-Ang2 full length antibody does not inhibit the binding between Ang2 and Tie2, wherein the first VEGFR component is fused to the C-terminus of a first heavy chain of the anti-Ang2 full length antibody via an optional first linker (e.g., SEQ ID NO: 30 or 31), and wherein the second VEGFR component is fused to the C-terminus of a second heavy chain of the anti-Ang2 full length antibody via an optional second linker (e.g., SEQ ID NO: 30 or 31). See, e.g., FIG. 6A. In some embodiments, the first and second linkers are the same. In some embodiments, the first and second linkers are different. In some embodiments, the first and second VEGFR components are the same. In some embodiments, the first and second VEGFR components are different. In some embodiments, the two heavy chain fusion polypeptides are the same. In some embodiments, there is provided an antibody fusion protein comprising: i) an anti-Ang2 full length antibody, ii) a first VEGFR component, and iii) a second VEGFR component, wherein the anti-Ang2 full length antibody does not inhibit the binding between Ang2 and Tie2, wherein the first VEGFR component is fused to the C-terminus of a first heavy chain of the anti-Ang2 full length antibody via a first linker, wherein the second VEGFR component is fused to the C-terminus of a second heavy chain of the anti-Ang2 full length antibody via a second linker, and wherein the antibody fusion protein comprises any of the following: (1) two heavy chain fusion polypeptides each comprising the amino acid sequence of SEQ ID NO: 50 or 52, and two light chains each comprising the amino acid sequence of SEQ ID NO: 42; (2) two heavy chain fusion polypeptides each comprising the amino acid sequence of SEQ ID NO: 51, and two light chains each comprising the amino acid sequence of SEQ ID NO: 41 or 42; (3) two heavy chain fusion polypeptides each comprising the amino acid sequence of SEQ ID NO: 59, and two light chains each comprising the amino acid sequence of SEQ ID NO: 45; (4) two heavy chain fusion polypeptides each comprising the amino acid sequence of SEQ ID NO: 60, and two light chains each comprising the amino acid sequence of SEQ ID NO: 46; (5) two heavy chain fusion polypeptides each comprising the amino acid sequence of SEQ ID NO: 61, and two light chains each comprising the amino acid sequence of SEQ ID NO: 44; (6) two heavy chain fusion polypeptides each comprising the amino acid sequence of SEQ ID NO: 75, and two light chains each comprising the amino acid sequence of SEQ ID NO: 47; or (7) two heavy chain fusion polypeptides each comprising the amino acid sequence of SEQ ID NO: 79, and two light chains each comprising the amino acid sequence of SEQ ID NO: 48. In some embodiments, the anti-Ang2 full length antibody upon binding to Ang2 activates Tie2 signaling through the antibody-bound Ang2.

In some embodiments, there is provided an antibody fusion protein comprising: i) an anti-Ang2 full length antibody, and ii) a VEGFR component (e.g., SEQ ID NO: 32), wherein the anti-Ang2 full length antibody does not inhibit the binding between Ang2 and Tie2, and wherein the VEGFR component is fused to the N-terminus of a light chain of the anti-Ang2 full length antibody via an optional linker (e.g., SEQ ID NO: 30 or 31). In some embodiments, the light chain fusion polypeptide comprises the amino acid sequence of any of SEQ ID NOs: 67-71, 73, 77, and 81. In some embodiments, there is provided an antibody fusion protein comprising: i) an anti-Ang2 full length antibody, and ii) a VEGFR component (e.g., SEQ ID NO: 32), wherein the anti-Ang2 full length antibody does not inhibit the binding between Ang2 and Tie2, wherein the VEGFR component is fused to the N-terminus of a light chain of the anti-Ang2 full length antibody via a linker, and wherein the antibody fusion protein comprises any of the following: (1) a heavy chain comprising the amino acid sequence of SEQ ID NO: 33, and a light chain fusion polypeptide comprising the amino acid sequence of SEQ ID NO: 67; (2) a heavy chain comprising the amino acid sequence of SEQ ID NO: 34, and a light chain fusion polypeptide comprising the amino acid sequence of any of SEQ ID NOs: 67, 68, and 77; (3) a heavy chain comprising the amino acid sequence of SEQ ID NO: 37, and a light chain fusion polypeptide comprising the amino acid sequence of SEQ ID NO: 69; (4) a heavy chain comprising the amino acid sequence of SEQ ID NO: 36, and a light chain fusion polypeptide comprising the amino acid sequence of SEQ ID NO: 70; (5) a heavy chain comprising the amino acid sequence of SEQ ID NO: 35, and a light chain fusion polypeptide comprising the amino acid sequence of SEQ ID NO: 71; (6) a heavy chain comprising the amino acid sequence of SEQ ID NO: 39, and a light chain fusion polypeptide comprising the amino acid sequence of SEQ ID NO: 73; (7) a heavy chain comprising the amino acid sequence of SEQ ID NO: 38, and a light chain fusion polypeptide comprising the amino acid sequence of SEQ ID NO: 67; or (8) a heavy chain comprising the amino acid sequence of SEQ ID NO: 40, and a light chain fusion polypeptide comprising the amino acid sequence of SEQ ID NO: 81. In some embodiments, there is provided an antibody fusion protein comprising: i) an anti-Ang2 full length antibody, and ii) a VEGFR component, wherein the anti-Ang2 full length antibody does not inhibit the binding between Ang2 and Tie2, wherein the VEGFR component is fused to the N-terminus of a light chain of the anti-Ang2 full length antibody via a linker, and wherein the antibody fusion protein comprises any of the following: (1) two heavy chains each comprising the amino acid sequence of SEQ ID NO: 33, a light chain comprising the amino acid sequence of SEQ ID NO: 42, and a light chain fusion polypeptide comprising the amino acid sequence of SEQ ID NO: 67; (2) two heavy chains each comprising the amino acid sequence of SEQ ID NO: 34, a light chain comprising the amino acid sequence of SEQ ID NO: 42, and a light chain fusion polypeptide comprising the amino acid sequence of SEQ ID NO: 67; (3) two heavy chains each comprising the amino acid sequence of SEQ ID NO: 34, a light chain comprising the amino acid sequence of SEQ ID NO: 41, and a light chain fusion polypeptide comprising the amino acid sequence of SEQ ID NO: 68; (4) two heavy chains each comprising the amino acid sequence of SEQ ID NO: 34, a light chain comprising the amino acid sequence of SEQ ID NO: 43, and a light chain fusion polypeptide comprising the amino acid sequence of SEQ ID NO: 77; (5) two heavy chains each comprising the amino acid sequence of SEQ ID NO: 37, a light chain comprising the amino acid sequence of SEQ ID NO: 45, and a light chain fusion polypeptide comprising the amino acid sequence of SEQ ID NO: 69; (6) two heavy chains each comprising the amino acid sequence of SEQ ID NO: 36, a light chain comprising the amino acid sequence of SEQ ID NO: 46, and a light chain fusion polypeptide comprising the amino acid sequence of SEQ ID NO: 70; (7) two heavy chains each comprising the amino acid sequence of SEQ ID NO: 35, a light chain comprising the amino acid sequence of SEQ ID NO: 44, and a light chain fusion polypeptide comprising the amino acid sequence of SEQ ID NO: 71; (8) two heavy chains each comprising the amino acid sequence of SEQ ID NO: 39, a light chain comprising the amino acid sequence of SEQ ID NO: 47, and a light chain fusion polypeptide comprising the amino acid sequence of SEQ ID NO: 73; (9) two heavy chains each comprising the amino acid sequence of SEQ ID NO: 38, a light chain comprising the amino acid sequence of SEQ ID NO: 42, and a light chain fusion polypeptide comprising the amino acid sequence of SEQ ID NO: 67; or (10) two heavy chains each comprising the amino acid sequence of SEQ ID NO: 40, a light chain comprising the amino acid sequence of SEQ ID NO: 48, and a light chain fusion polypeptide comprising the amino acid sequence of SEQ ID NO: 81. In some embodiments, the anti-Ang2 full length antibody upon binding to Ang2 activates Tie2 signaling through the antibody-bound Ang2.

In some embodiments, there is provided an antibody fusion protein comprising: i) an anti-Ang2 full length antibody, ii) a first VEGFR component (e.g., SEQ ID NO: 32), and iii) a second VEGFR component (e.g., SEQ ID NO: 32), wherein the anti-Ang2 full length antibody does not inhibit the binding between Ang2 and Tie2, wherein the first VEGFR component is fused to the N-terminus of a first light chain of the anti-Ang2 full length antibody via an optional first linker (e.g., SEQ ID NO: 30 or 31), and wherein the second VEGFR component is fused to the N-terminus of a second light chain of the anti-Ang2 full length antibody via an optional second linker (e.g., SEQ ID NO: 30 or 31). See, e.g., FIG. 6D. In some embodiments, the first and second linkers are the same. In some embodiments, the first and second linkers are different. In some embodiments, the first and second VEGFR components are the same. In some embodiments, the first and second VEGFR components are different. In some embodiments, the two light chain fusion polypeptides are the same. In some embodiments, there is provided an antibody fusion protein comprising: i) an anti-Ang2 full length antibody, ii) a first VEGFR component, and iii) a second VEGFR component, wherein the anti-Ang2 full length antibody does not inhibit the binding between Ang2 and Tie2, wherein the first VEGFR component is fused to the N-terminus of a first light chain of the anti-Ang2 full length antibody via a first linker, wherein the second VEGFR component is fused to the N-terminus of a second light chain of the anti-Ang2 full length antibody via a second linker, and wherein the antibody fusion protein comprises any of the following: (1) two heavy chains each comprising the amino acid sequence of SEQ ID NO: 33, and two light chain fusion polypeptides each comprising the amino acid sequence of SEQ ID NO: 67; (2) two heavy chains each comprising the amino acid sequence of SEQ ID NO: 34, and two light chain fusion polypeptides each comprising the amino acid sequence of any of SEQ ID NOs: 67, 68, and 77; (3) two heavy chains each comprising the amino acid sequence of SEQ ID NO: 37, and two light chain fusion polypeptides each comprising the amino acid sequence of SEQ ID NO: 69; (4) two heavy chains each comprising the amino acid sequence of SEQ ID NO: 36, and two light chain fusion polypeptides each comprising the amino acid sequence of SEQ ID NO: 70; (5) two heavy chains each comprising the amino acid sequence of SEQ ID NO: 35, and two light chain fusion polypeptides each comprising the amino acid sequence of SEQ ID NO: 71; (6) two heavy chains each comprising the amino acid sequence of SEQ ID NO: 39, and two light chain fusion polypeptides each comprising the amino acid sequence of SEQ ID NO: 73; (7) two heavy chains each comprising the amino acid sequence of SEQ ID NO: 38, and two light chain fusion polypeptides each comprising the amino acid sequence of SEQ ID NO: 67; or (8) two heavy chains each comprising the amino acid sequence of SEQ ID NO: 40, and two light chain fusion polypeptides each comprising the amino acid sequence of SEQ ID NO: 81. In some embodiments, the anti-Ang2 full length antibody upon binding to Ang2 activates Tie2 signaling through the antibody-bound Ang2.

In some embodiments, there is provided an antibody fusion protein comprising: i) an anti-Ang2 full length antibody, and ii) a VEGFR component (e.g., SEQ ID NO: 32), wherein the anti-Ang2 full length antibody does not inhibit the binding between Ang2 and Tie2, and wherein the VEGFR component is fused to the C-terminus of a light chain of the anti-Ang2 full length antibody via an optional linker (e.g., SEQ ID NO: 30 or 31). In some embodiments, the light chain fusion polypeptide comprises the amino acid sequence of any of SEQ ID NOs: 62-66, 72, 76, and 80. In some embodiments, there is provided an antibody fusion protein comprising: i) an anti-Ang2 full length antibody, and ii) a VEGFR component, wherein the anti-Ang2 full length antibody does not inhibit the binding between Ang2 and Tie2, wherein the VEGFR component is fused to the C-terminus of a light chain of the anti-Ang2 full length antibody via a linker, and wherein the antibody fusion protein comprises any of the following: (1) a heavy chain comprising the amino acid sequence of SEQ ID NO: 33, and a light chain fusion polypeptide comprising the amino acid sequence of SEQ ID NO: 62; (2) a heavy chain comprising the amino acid sequence of SEQ ID NO: 34, and a light chain fusion polypeptide comprising the amino acid sequence of any of SEQ ID NOs: 62, 63, and 76; (3) a heavy chain comprising the amino acid sequence of SEQ ID NO: 37, and a light chain fusion polypeptide comprising the amino acid sequence of SEQ ID NO: 64; (4) a heavy chain comprising the amino acid sequence of SEQ ID NO: 36, and a light chain fusion polypeptide comprising the amino acid sequence of SEQ ID NO: 65; (5) a heavy chain comprising the amino acid sequence of SEQ ID NO: 35, and a light chain fusion polypeptide comprising the amino acid sequence of SEQ ID NO: 66; (6) a heavy chain comprising the amino acid sequence of SEQ ID NO: 39, and a light chain fusion polypeptide comprising the amino acid sequence of SEQ ID NO: 72; (7) a heavy chain comprising the amino acid sequence of SEQ ID NO: 38, and a light chain fusion polypeptide comprising the amino acid sequence of SEQ ID NO: 62; or (8) a heavy chain comprising the amino acid sequence of SEQ ID NO: 40, and a light chain fusion polypeptide comprising the amino acid sequence of SEQ ID NO: 80. In some embodiments, there is provided an antibody fusion protein comprising: i) an anti-Ang2 full length antibody, and ii) a VEGFR component, wherein the anti-Ang2 full length antibody does not inhibit the binding between Ang2 and Tie2, wherein the VEGFR component is fused to the C-terminus of a light chain of the anti-Ang2 full length antibody via a linker, and wherein the antibody fusion protein comprises any of the following: (1) two heavy chains each comprising the amino acid sequence of SEQ ID NO: 33, a light chain comprising the amino acid sequence of SEQ ID NO: 42, and a light chain fusion polypeptide comprising the amino acid sequence of SEQ ID NO: 62; (2) two heavy chains each comprising the amino acid sequence of SEQ ID NO: 34, a light chain comprising the amino acid sequence of SEQ ID NO: 42, and a light chain fusion polypeptide comprising the amino acid sequence of SEQ ID NO: 62; (3) two heavy chains each comprising the amino acid sequence of SEQ ID NO: 34, a light chain comprising the amino acid sequence of SEQ ID NO: 41, and a light chain fusion polypeptide comprising the amino acid sequence of SEQ ID NO: 63; (4) two heavy chains each comprising the amino acid sequence of SEQ ID NO: 34, a light chain comprising the amino acid sequence of SEQ ID NO: 43, and a light chain fusion polypeptide comprising the amino acid sequence of SEQ ID NO: 76; (5) two heavy chains each comprising the amino acid sequence of SEQ ID NO: 37, a light chain comprising the amino acid sequence of SEQ ID NO: 45, and a light chain fusion polypeptide comprising the amino acid sequence of SEQ ID NO: 64; (6) two heavy chains each comprising the amino acid sequence of SEQ ID NO: 36, a light chain comprising the amino acid sequence of SEQ ID NO: 46, and a light chain fusion polypeptide comprising the amino acid sequence of SEQ ID NO: 65; (7) two heavy chains each comprising the amino acid sequence of SEQ ID NO: 35, a light chain comprising the amino acid sequence of SEQ ID NO: 44, and a light chain fusion polypeptide comprising the amino acid sequence of SEQ ID NO: 66; (8) two heavy chains each comprising the amino acid sequence of SEQ ID NO: 39, a light chain comprising the amino acid sequence of SEQ ID NO: 47, and a light chain fusion polypeptide comprising the amino acid sequence of SEQ ID NO: 72; (9) two heavy chains each comprising the amino acid sequence of SEQ ID NO: 38, a light chain comprising the amino acid sequence of SEQ ID NO: 42, and a light chain fusion polypeptide comprising the amino acid sequence of SEQ ID NO: 62; or (10) two heavy chains each comprising the amino acid sequence of SEQ ID NO: 40, a light chain comprising the amino acid sequence of SEQ ID NO: 48, and a light chain fusion polypeptide comprising the amino acid sequence of SEQ ID NO: 80. In some embodiments, the anti-Ang2 full length antibody upon binding to Ang2 activates Tie2 signaling through the antibody-bound Ang2.

In some embodiments, there is provided an antibody fusion protein comprising: i) an anti-Ang2 full length antibody, ii) a first VEGFR component (e.g., SEQ ID NO: 32), and iii) a second VEGFR component (e.g., SEQ ID NO: 32), wherein the anti-Ang2 full length antibody does not inhibit the binding between Ang2 and Tie2, wherein the first VEGFR component is fused to the C-terminus of a first light chain of the anti-Ang2 full length antibody via an optional first linker (e.g., SEQ ID NO: 30 or 31), and wherein the second VEGFR component is fused to the C-terminus of a second light chain of the anti-Ang2 full length antibody via an optional second linker (e.g., SEQ ID NO: 30 or 31). See, e.g., FIG. 6B. In some embodiments, the first and second linkers are the same. In some embodiments, the first and second linkers are different. In some embodiments, the first and second VEGFR components are the same. In some embodiments, the first and second VEGFR components are different. In some embodiments, the two light chain fusion polypeptides are the same. In some embodiments, there is provided an antibody fusion protein comprising: i) an anti-Ang2 full length antibody, ii) a first VEGFR component, and iii) a second VEGFR component, wherein the anti-Ang2 full length antibody does not inhibit the binding between Ang2 and Tie2, wherein the first VEGFR component is fused to the C-terminus of a first light chain of the anti-Ang2 full length antibody via a first linker, wherein the second VEGFR component is fused to the C-terminus of a second light chain of the anti-Ang2 full length antibody via a second linker, and wherein the antibody fusion protein comprises any of the following: (1) two heavy chains each comprising the amino acid sequence of SEQ ID NO: 33, and two light chain fusion polypeptides each comprising the amino acid sequence of SEQ ID NO: 62; (2) two heavy chains each comprising the amino acid sequence of SEQ ID NO: 34, and two light chain fusion polypeptides each comprising the amino acid sequence of any of SEQ ID NOs: 62, 63, and 76; (3) two heavy chains each comprising the amino acid sequence of SEQ ID NO: 37, and two light chain fusion polypeptides each comprising the amino acid sequence of SEQ ID NO: 64; (4) two heavy chains each comprising the amino acid sequence of SEQ ID NO: 36, and two light chain fusion polypeptides each comprising the amino acid sequence of SEQ ID NO: 65; (5) two heavy chains each comprising the amino acid sequence of SEQ ID NO: 35, and two light chain fusion polypeptides each comprising the amino acid sequence of SEQ ID NO: 66; (6) two heavy chains each comprising the amino acid sequence of SEQ ID NO: 39, and two light chain fusion polypeptides each comprising the amino acid sequence of SEQ ID NO: 72; (7) two heavy chains each comprising the amino acid sequence of SEQ ID NO: 38, and two light chain fusion polypeptides each comprising the amino acid sequence of SEQ ID NO: 62; or (8) two heavy chains each comprising the amino acid sequence of SEQ ID NO: 40, and two light chain fusion polypeptides each comprising the amino acid sequence of SEQ ID NO: 80. In some embodiments, the anti-Ang2 full length antibody upon binding to Ang2 activates Tie2 signaling through the antibody-bound Ang2.

In some embodiments, there is provided an antibody fusion protein comprising: i) an anti-Ang2 full length antibody, and ii) four VEGFR components (e.g., SEQ ID NO: 32), wherein the anti-Ang2 full length antibody does not inhibit the binding between Ang2 and Tie2, wherein the first VEGFR component is fused to the N-terminus of a first heavy chain of the anti-Ang2 full length antibody via an optional first linker, wherein the second VEGFR component is fused to the N-terminus of a second heavy chain of the anti-Ang2 full length antibody via an optional second linker, wherein the third VEGFR component is fused to the N-terminus of a first light chain of the anti-Ang2 full length antibody via an optional third linker, and wherein the fourth VEGFR component is fused to the N-terminus of a second light chain of the anti-Ang2 full length antibody via an optional fourth linker. See, e.g., FIG. 6E. In some embodiments, there is provided an antibody fusion protein comprising: i) an anti-Ang2 full length antibody, and ii) four VEGFR components (e.g., SEQ ID NO: 32), wherein the anti-Ang2 full length antibody does not inhibit the binding between Ang2 and Tie2, wherein the first VEGFR component is fused to the C-terminus of a first heavy chain of the anti-Ang2 full length antibody via an optional first linker, wherein the second VEGFR component is fused to the C-terminus of a second heavy chain of the anti-Ang2 full length antibody via an optional second linker, wherein the third VEGFR component is fused to the C-terminus of a first light chain of the anti-Ang2 full length antibody via an optional third linker, and wherein the fourth VEGFR component is fused to the C-terminus of a second light chain of the anti-Ang2 full length antibody via an optional fourth linker. See, e.g., FIG. 6F. In some embodiments, there is provided an antibody fusion protein comprising: i) an anti-Ang2 full length antibody, and ii) four VEGFR components (e.g., SEQ ID NO: 32), wherein the anti-Ang2 full length antibody does not inhibit the binding between Ang2 and Tie2, wherein the first VEGFR component is fused to the N-terminus of a first heavy chain of the anti-Ang2 full length antibody via an optional first linker, wherein the second VEGFR component is fused to the N-terminus of a second heavy chain of the anti-Ang2 full length antibody via an optional second linker, wherein the third VEGFR component is fused to the C-terminus of the first heavy chain of the anti-Ang2 full length antibody via an optional third linker, and wherein the fourth VEGFR component is fused to the C-terminus of the second heavy chain of the anti-Ang2 full length antibody via an optional fourth linker. See, e.g., FIG. 6G. In some embodiments, there is provided an antibody fusion protein comprising: i) an anti-Ang2 full length antibody, and ii) four VEGFR components (e.g., SEQ ID NO: 32), wherein the anti-Ang2 full length antibody does not inhibit the binding between Ang2 and Tie2, wherein the first VEGFR component is fused to the C-terminus of a first heavy chain of the anti-Ang2 full length antibody via an optional first linker, wherein the second VEGFR component is fused to the C-terminus of a second heavy chain of the anti-Ang2 full length antibody via an optional second linker, wherein the third VEGFR component is fused to the N-terminus of a first light chain of the anti-Ang2 full length antibody via an optional third linker, and wherein the fourth VEGFR component is fused to the N-terminus of a second light chain of the anti-Ang2 full length antibody via an optional fourth linker. See, e.g., FIG. 6H. In some embodiments, there is provided an antibody fusion protein comprising: i) an anti-Ang2 full length antibody, and ii) four VEGFR components (e.g., SEQ ID NO: 32), wherein the anti-Ang2 full length antibody does not inhibit the binding between Ang2 and Tie2, wherein the first VEGFR component is fused to the N-terminus of a first heavy chain of the anti-Ang2 full length antibody via an optional first linker, wherein the second VEGFR component is fused to the N-terminus of a second heavy chain of the anti-Ang2 full length antibody via an optional second linker, wherein the third VEGFR component is fused to the C-terminus of a first light chain of the anti-Ang2 full length antibody via an optional third linker, and wherein the fourth VEGFR component is fused to the C-terminus of a second light chain of the anti-Ang2 full length antibody via an optional fourth linker. See, e.g., FIG. 6I. In some embodiments, there is provided an antibody fusion protein comprising: i) an anti-Ang2 full length antibody, and ii) four VEGFR components (e.g., SEQ ID NO: 32), wherein the anti-Ang2 full length antibody does not inhibit the binding between Ang2 and Tie2, wherein the first VEGFR component is fused to the N-terminus of a first light chain of the anti-Ang2 full length antibody via an optional first linker, wherein the second VEGFR component is fused to the N-terminus of a second light chain of the anti-Ang2 full length antibody via an optional second linker, wherein the third VEGFR component is fused to the C-terminus of the first light chain of the anti-Ang2 full length antibody via an optional third linker, and wherein the fourth VEGFR component is fused to the C-terminus of the second light chain of the anti-Ang2 full length antibody via an optional fourth linker. See, e.g., FIG. 6J. In some embodiments, the anti-Ang2 full length antibody upon binding to Ang2 activates Tie2 signaling through the antibody-bound Ang2. In some embodiments, the first, second, third, and fourth linkers are all the same. In some embodiments, at least one of the first, second, third, and fourth linkers is different from the others. In some embodiments, the first, second, third, and fourth linkers are all different from each other. In some embodiments, the first and second linkers are the same. In some embodiments, the third and fourth linkers are the same. In some embodiments, the first, second, third, and fourth linkers each comprises an amino acid sequence independently selected from SEQ ID NO: 30 or 31. In some embodiments, the first and second VEGFR components are the same. In some embodiments, the third and fourth VEGFR components are the same. In some embodiments, all four VEGFR components are the same. In some embodiments, at least one VEGFR component is different from the others. In some embodiments, the four VEGFR components are different from each other.

### Multivalent Anti Ang2 Antibodies or Antigen-Binding Fragments Thereof

Multivalent (e.g., bivalent) anti-Ang2 antibodies or antigen-binding fragments thereof described herein (or the anti-Ang2/VEGF-trap antibody fusion protein) have one or more of the following properties: i) do not inhibit the binding between Ang2 and Tie2; ii) can specifically bind to Ang2 and trigger clustering of Ang2, assembling an antibody/Ang2 complex; iii) trigger Tie2 clustering by antibody-bound Ang2 or antibody/Ang2 complex; iv) activate Tie2 signaling by antibody-bound Ang2 or antibody/Ang2 complex; or v) reduce (e.g., reduce at least about any of 5%, 10%, 20%, 30%, 40%, 50%, 60%, 70%, 80%, 90%, 95%, or 100%) the competition/antagonist activity of Ang2 in preventing/reducing Ang1/Tie2 binding. See, e.g., FIG. 7C.

Binding specificity and/or affinity of a multivalent anti-Ang2 antibody or antigen-binding fragment thereof (such as a multivalent anti-Ang2 antibody or antigen-binding fragment thereof of the antibody fusion protein) to Ang2 can be determined experimentally by methods known in the art. Such methods comprise, but are not limited to Western blots, ELISA, MSD electrochemiluminescence, bead based MIA, RIA, SPR, ECL, IRMA, EIA, Biacore assay, Octet analysis, peptide scans, FACS, etc.

In some embodiments, the multivalent (e.g., bivalent) anti-Ang2 antibody or antigen-binding fragment thereof does not inhibit the binding between Ang2 and Tie2, for example, the binding affinity (e.g., K_{D}) between Ang2 and Tie2 in the presence of the multivalent anti-Ang2 antibody or antigen-binding fragment thereof (or the anti-Ang2/VEGF-trap antibody fusion protein) is the same as, or within about 2-fold difference of, the binding affinity (e.g., K_{D}) between Ang2 and Tie2 in the absence of the multivalent anti-Ang2 antibody or antigen-binding fragment thereof (or the anti-Ang2/VEGF-trap antibody fusion protein). In some embodiments, the multivalent (e.g., bivalent) anti-Ang2 antibody or antigen-binding fragment thereof reduces but not completely blocks/abolishes the binding between Ang2 and Tie2, for example, the binding between Ang2 and Tie2 is reduced at least about 3%, such as reduced at least about any of 4%, 5%, 6%, 7%, 8%, 9%, 10%, 20%, 30%, 40%, 50%, 60%, 70%, 80%, 90%, 95%, or more (but less than 100%), in the presence of the multivalent anti-Ang2 antibody or antigen-binding fragment thereof (or the anti-Ang2/VEGF-trap antibody fusion protein). In some embodiments, the multivalent (e.g., bivalent) anti-Ang2 antibody or antigen-binding fragment thereof increases (e.g., increasing at least about 2-fold) the binding between Ang2 and Tie2 in the presence of the multivalent anti-Ang2 antibody or antigen-binding fragment thereof (or the anti-Ang2/VEGF-trap antibody fusion protein).

In some embodiments, fusing the VEGFR component(s) to the multivalent anti-Ang2 antibody or antigen-binding fragment thereof does not affect the binding affinity and/or specificity of the multivalent anti-Ang2 antibody or antigen-binding fragment thereof to Ang2, for example, the K_{D} of the binding between the VEGFR component(s)-fused multivalent anti-Ang2 antibody or antigen-binding fragment thereof and Ang2 is the same as, or within about 2-fold difference of, the K_{D} of the binding between a corresponding non-fused multivalent anti-Ang2 antibody or antigen-binding fragment thereof and Ang2. In some embodiments, fusing the VEGFR component(s) to the multivalent anti-Ang2 antibody or antigen-binding fragment thereof reduces the binding affinity and/or specificity of the multivalent anti-Ang2 antibody or antigen-binding fragment thereof to Ang2, for example, the K_{D} of the binding between the VEGFR component(s)-fused multivalent anti-Ang2 antibody or antigen-binding fragment thereof and Ang2 is more than about 2 times of the K_{D} of the binding between a corresponding non-fused multivalent anti-Ang2 antibody or antigen-binding fragment thereof and Ang2. In some embodiments, fusing the VEGFR component(s) to the multivalent anti-Ang2 antibody or antigen-binding fragment thereof increases the binding affinity and/or specificity of the multivalent anti-Ang2 antibody or antigen-binding fragment thereof to Ang2, for example, the K_{D} of the binding between a corresponding non-fused multivalent anti-Ang2 antibody or antigen-binding fragment thereof and Ang2 is more than about 2 times of the K_{D} of the binding between the VEGFR component(s)-fused multivalent anti-Ang2 antibody or antigen-binding fragment thereof and Ang2.

The biological activity of a multivalent anti-Ang2 antibody or antigen-binding fragment thereof (such as multivalent anti-Ang2 antibody or antigen-binding fragment thereof of the antibody fusion protein) can be determined by any methods known in the art, such as Tie2-dependent receptor phosphorylation assay (e.g., using HUVECs, detecting Akt phosphorylation on Ser), Tie2-dependent reporter gene expression assay (e.g., using a cell that expresses Tie2 on the cell surface and carries a reporter gene under the control of Tie2 activation), or angiogesis assays such as tube or tube-like structure formation assay (e.g., using HUVECs or IBE cell line) or sprouting assay. Also see Example 1 for exemplary methods.

In some embodiments, fusing the VEGFR component(s) to the multivalent anti-Ang2 antibody or antigen-binding fragment thereof does not affect the biological activity of the multivalent anti-Ang2 antibody or antigen-binding fragment thereof, for example, the biological activity (e.g., binding to Ang2 and activating Tie2 via the antibody-bound Ang2) of the VEGFR component(s)-fused multivalent anti-Ang2 antibody or antigen-binding fragment thereof is the same as, or within about 2-fold difference of, the biological activity of a corresponding non-fused multivalent anti-Ang2 antibody or antigen-binding fragment thereof. In some embodiments, fusing the VEGFR component(s) to the multivalent anti-Ang2 antibody or antigen-binding fragment thereof reduces (e.g., reducing at least about 2-fold) the biological activity of the multivalent anti-Ang2 antibody or antigen-binding fragment thereof compared to a corresponding non-fused multivalent anti-Ang2 antibody or antigen-binding fragment thereof. In some embodiments, fusing the VEGFR component(s) to the multivalent anti-Ang2 antibody or antigen-binding fragment thereof increases (e.g., increasing at least about 2-fold) the biological activity of the multivalent anti-Ang2 antibody or antigen-binding fragment thereof compared to a corresponding non-fused multivalent anti-Ang2 antibody or antigen-binding fragment thereof.

In some embodiments, the multivalent (e.g., bivalent) anti-Ang2 antibody or antigen-binding fragment thereof (or the anti-Ang2/VEGF-trap antibody fusion protein) upon binding to Ang2 activates Tie2 signaling through the antibody-bound Ang2. In some embodiments, activated Tie2 signaling can achieve one or more of: i) activating the promigratory, prosurvival PI3K/Akt pathway (e.g., in endothelial cells); ii) stabilizing vasculature and/or lymphatic vessels, blunting vascular leakage; iii) strengthening endothelial barrier; iv) protecting endothelial glycocalyx; or v) reducing inflammation.

The multivalent (e.g., bivalent) anti-Ang2 antibody or antigen-binding fragment thereof can be of any possible format. In some embodiments, the multivalent anti-Ang2 antibody or antigen-binding fragment thereof comprises a single polypeptide chain. In some embodiments, the multivalent anti-Ang2 antibody or antigen-binding fragment thereof comprises more than one (such as any of 2, 3, 4, or more) polypeptide chains. In the cases of multi-chain antibodies or antigen-binding fragments thereof, the nucleic acid sequences encoding the polypeptide chains may be operably linked to the same promoter or to different promoters. In some embodiments, the multivalent anti-Ang2 antibody or antigen-binding fragment thereof comprises one or more constant domains, such as C_{L}, C_{H}1, C_{H}2, C_{H}3, or any combination thereof. In some embodiments, the multivalent anti-Ang2 antibody or antigen-binding fragment thereof comprises an Fc domain or portion thereof (e.g., CH2, CH3, CH2+CH3, or portion(s) thereof). In some embodiments, the multivalent anti-Ang2 antibody or antigen-binding fragment thereof does not comprise an Fc domain or portion thereof, e.g., F(ab′)₂. In some embodiments, the multivalent anti-Ang2 antibody or antigen-binding fragment thereof is a full length antibody (“anti-Ang2 full length antibody”). In some embodiments, the anti-Ang2 full length antibody comprises a CL comprising the amino acid sequence of SEQ ID NO: 100. In some embodiments, the anti-Ang2 full length antibody comprises an Fc domain comprising the amino acid sequence of SEQ ID NO: 99. In some embodiments, the multivalent anti-Ang2 antibody or antigen-binding fragment thereof is a monoclonal antibody. In some embodiments, the anti-Ang2 binding domain(s) of the multivalent anti-Ang2 antibody or antigen-binding fragment thereof can be independently selected from any of Fab, Fab′, minibody, sdAb, Fv, scFv, etc. In some embodiments, the multivalent anti-Ang2 antibody or antigen-binding fragment thereof is human, humanized, chimeric, or from non-human source (e.g., murine, rat, rabbit, hamster, guinea pig, sheep, horse, pig, cow, birds, etc.).

The multivalent anti-Ang2 antibody or antigen-binding fragment thereof can have 2, 3, 4, 5, or more anti-Ang2 valency or anti-Ang2 binding domains. In some embodiments, the multivalent (e.g., bivalent) anti-Ang2 antibody (e.g., full length antibody) or antigen-binding fragment thereof is monospecific. In some embodiments, the multivalent (e.g., bivalent) anti-Ang2 antibody (e.g., full length antibody) or antigen-binding fragment thereof is multispecific (e.g., bispecific). In some embodiments, the valency number (e.g., bivalent) and the specificity number (e.g., bispecific) of the multivalent anti-Ang2 antibody or antigen-binding fragment thereof are the same. In some embodiments, the valency number (e.g., trivalent) is more than the specificity number (e.g., monospecific) of the multivalent anti-Ang2 antibody or antigen-binding fragment thereof.

In some embodiments, the multivalent (e.g., bivalent) anti-Ang2 antibody (e.g., full length antibody) or antigen-binding fragment thereof comprises i) an HC-CDR1 comprising the amino acid sequence of any one of SEQ ID NOs: 1-6, or a variant thereof comprising up to about 3 (e.g., 1, 2, or 3) amino acid substitutions; ii) an HC-CDR2 comprising the amino acid sequence of any one of SEQ ID NOs: 7-11, or a variant thereof comprising up to about 3 (e.g., 1, 2, or 3) amino acid substitutions; iii) an HC-CDR3 comprising the amino acid sequence of any one of SEQ ID NOs: 12-16, or a variant thereof comprising up to about 3 (e.g., 1, 2, or 3) amino acid substitutions; iv) an LC-CDR1 comprising the amino acid sequence of SEQ ID NO: 17 or 18, or a variant thereof comprising up to about 3 (e.g., 1, 2, or 3) amino acid substitutions; v) an LC-CDR2 comprising the amino acid sequence of any one of SEQ ID NOs: 19-23, or a variant thereof comprising up to about 3 (e.g., 1, 2, or 3) amino acid substitutions; and vi) an LC-CDR3 comprising the amino acid sequence of any one of SEQ ID NOs: 24-28, or a variant thereof comprising up to about 3 (e.g., 1, 2, or 3) amino acid substitutions. In some embodiments, the multivalent (e.g., bivalent) anti-Ang2 antibody (e.g., full length antibody) or antigen-binding fragment thereof comprises i) an HC-CDR1 comprising the amino acid sequence of any one of SEQ ID NOs: 1-6; ii) an HC-CDR2 comprising the amino acid sequence of any one of SEQ ID NOs: 7-11; iii) an HC-CDR3 comprising the amino acid sequence of any one of SEQ ID NOs: 12-16; iv) an LC-CDR1 comprising the amino acid sequence of SEQ ID NO: 17 or 18; v) an LC-CDR2 comprising the amino acid sequence of any one of SEQ ID NOs: 19-23; and vi) an LC-CDR3 comprising the amino acid sequence of any one of SEQ ID NOs: 24-28; or a variant thereof comprising up to about 3 (e.g., 1, 2, or 3) amino acid substitutions in any of the HC-CDRs and LC-CDRs. In some embodiments, the multivalent (e.g., bivalent) anti-Ang2 antibody (e.g., full length antibody) or antigen-binding fragment thereof comprises i) an HC-CDR1 comprising the amino acid sequence of any one of SEQ ID NOs: 1-6; ii) an HC-CDR2 comprising the amino acid sequence of any one of SEQ ID NOs: 7-11; iii) an HC-CDR3 comprising the amino acid sequence of any one of SEQ ID NOs: 12-16; iv) an LC-CDR1 comprising the amino acid sequence of SEQ ID NO: 17 or 18; v) an LC-CDR2 comprising the amino acid sequence of any one of SEQ ID NOs: 19-23; and vi) an LC-CDR3 comprising the amino acid sequence of any one of SEQ ID NOs: 24-28.

In some embodiments, the multivalent (e.g., bivalent) anti-Ang2 antibody (e.g., full length antibody) or antigen-binding fragment thereof comprises any of the following: (1) i) an HC-CDR1 comprising the amino acid sequence of SEQ ID NO: 1, or a variant thereof comprising up to about 3 (e.g., 1, 2, or 3) amino acid substitutions; ii) an HC-CDR2 comprising the amino acid sequence of SEQ ID NO: 7, or a variant thereof comprising up to about 3 (e.g., 1, 2, or 3) amino acid substitutions; iii) an HC-CDR3 comprising the amino acid sequence of SEQ ID NO: 12, or a variant thereof comprising up to about 3 (e.g., 1, 2, or 3) amino acid substitutions; iv) an LC-CDR1 comprising the amino acid sequence of SEQ ID NO: 17, or a variant thereof comprising up to about 3 (e.g., 1, 2, or 3) amino acid substitutions; v) an LC-CDR2 comprising the amino acid sequence of SEQ ID NO: 19, or a variant thereof comprising up to about 3 (e.g., 1, 2, or 3) amino acid substitutions; and vi) an LC-CDR3 comprising the amino acid sequence of SEQ ID NO: 24, or a variant thereof comprising up to about 3 (e.g., 1, 2, or 3) amino acid substitutions; (2) i) an HC-CDR1 comprising the amino acid sequence of SEQ ID NO: 2, or a variant thereof comprising up to about 3 (e.g., 1, 2, or 3) amino acid substitutions; ii) an HC-CDR2 comprising the amino acid sequence of SEQ ID NO: 8, or a variant thereof comprising up to about 3 (e.g., 1, 2, or 3) amino acid substitutions; iii) an HC-CDR3 comprising the amino acid sequence of SEQ ID NO: 13, or a variant thereof comprising up to about 3 (e.g., 1, 2, or 3) amino acid substitutions; iv) an LC-CDR1 comprising the amino acid sequence of SEQ ID NO: 17, or a variant thereof comprising up to about 3 (e.g., 1, 2, or 3) amino acid substitutions; v) an LC-CDR2 comprising the amino acid sequence of SEQ ID NO: 20, or a variant thereof comprising up to about 3 (e.g., 1, 2, or 3) amino acid substitutions; and vi) an LC-CDR3 comprising the amino acid sequence of SEQ ID NO: 25, or a variant thereof comprising up to about 3 (e.g., 1, 2, or 3) amino acid substitutions; (3) i) an HC-CDR1 comprising the amino acid sequence of SEQ ID NO: 3, or a variant thereof comprising up to about 3 (e.g., 1, 2, or 3) amino acid substitutions; ii) an HC-CDR2 comprising the amino acid sequence of SEQ ID NO: 9, or a variant thereof comprising up to about 3 (e.g., 1, 2, or 3) amino acid substitutions; iii) an HC-CDR3 comprising the amino acid sequence of SEQ ID NO: 14, or a variant thereof comprising up to about 3 (e.g., 1, 2, or 3) amino acid substitutions; iv) an LC-CDR1 comprising the amino acid sequence of SEQ ID NO: 17, or a variant thereof comprising up to about 3 (e.g., 1, 2, or 3) amino acid substitutions; v) an LC-CDR2 comprising the amino acid sequence of SEQ ID NO: 21, or a variant thereof comprising up to about 3 (e.g., 1, 2, or 3) amino acid substitutions; and vi) an LC-CDR3 comprising the amino acid sequence of SEQ ID NO: 26, or a variant thereof comprising up to about 3 (e.g., 1, 2, or 3) amino acid substitutions; (4) i) an HC-CDR1 comprising the amino acid sequence of SEQ ID NO: 4, or a variant thereof comprising up to about 3 (e.g., 1, 2, or 3) amino acid substitutions; ii) an HC-CDR2 comprising the amino acid sequence of SEQ ID NO: 7, or a variant thereof comprising up to about 3 (e.g., 1, 2, or 3) amino acid substitutions; iii) an HC-CDR3 comprising the amino acid sequence of SEQ ID NO: 12, or a variant thereof comprising up to about 3 (e.g., 1, 2, or 3) amino acid substitutions; iv) an LC-CDR1 comprising the amino acid sequence of SEQ ID NO: 17, or a variant thereof comprising up to about 3 (e.g., 1, 2, or 3) amino acid substitutions; v) an LC-CDR2 comprising the amino acid sequence of SEQ ID NO: 19, or a variant thereof comprising up to about 3 (e.g., 1, 2, or 3) amino acid substitutions; and vi) an LC-CDR3 comprising the amino acid sequence of SEQ ID NO: 24, or a variant thereof comprising up to about 3 (e.g., 1, 2, or 3) amino acid substitutions; (5) i) an HC-CDR1 comprising the amino acid sequence of SEQ ID NO: 5, or a variant thereof comprising up to about 3 (e.g., 1, 2, or 3) amino acid substitutions; ii) an HC-CDR2 comprising the amino acid sequence of SEQ ID NO: 10, or a variant thereof comprising up to about 3 (e.g., 1, 2, or 3) amino acid substitutions; iii) an HC-CDR3 comprising the amino acid sequence of SEQ ID NO: 15, or a variant thereof comprising up to about 3 (e.g., 1, 2, or 3) amino acid substitutions; iv) an LC-CDR1 comprising the amino acid sequence of SEQ ID NO: 18, or a variant thereof comprising up to about 3 (e.g., 1, 2, or 3) amino acid substitutions; v) an LC-CDR2 comprising the amino acid sequence of SEQ ID NO: 22, or a variant thereof comprising up to about 3 (e.g., 1, 2, or 3) amino acid substitutions; and vi) an LC-CDR3 comprising the amino acid sequence of SEQ ID NO: 27, or a variant thereof comprising up to about 3 (e.g., 1, 2, or 3) amino acid substitutions; or (6) i) an HC-CDR1 comprising the amino acid sequence of SEQ ID NO: 6, or a variant thereof comprising up to about 3 (e.g., 1, 2, or 3) amino acid substitutions; ii) an HC-CDR2 comprising the amino acid sequence of SEQ ID NO: 11, or a variant thereof comprising up to about 3 (e.g., 1, 2, or 3) amino acid substitutions; iii) an HC-CDR3 comprising the amino acid sequence of SEQ ID NO: 16, or a variant thereof comprising up to about 3 (e.g., 1, 2, or 3) amino acid substitutions; iv) an LC-CDR1 comprising the amino acid sequence of SEQ ID NO: 17, or a variant thereof comprising up to about 3 (e.g., 1, 2, or 3) amino acid substitutions; v) an LC-CDR2 comprising the amino acid sequence of SEQ ID NO: 23, or a variant thereof comprising up to about 3 (e.g., 1, 2, or 3) amino acid substitutions; and vi) an LC-CDR3 comprising the amino acid sequence of SEQ ID NO: 28, or a variant thereof comprising up to about 3 (e.g., 1, 2, or 3) amino acid substitutions. In some embodiments, the multivalent (e.g., bivalent) anti-Ang2 antibody (e.g., full length antibody) or antigen-binding fragment thereof comprises any of the following: (1) i) an HC-CDR1 comprising the amino acid sequence of SEQ ID NO: 1; ii) an HC-CDR2 comprising the amino acid sequence of SEQ ID NO: 7; iii) an HC-CDR3 comprising the amino acid sequence of SEQ ID NO: 12; iv) an LC-CDR1 comprising the amino acid sequence of SEQ ID NO: 17; v) an LC-CDR2 comprising the amino acid sequence of SEQ ID NO: 19; and vi) an LC-CDR3 comprising the amino acid sequence of SEQ ID NO: 24; or a variant thereof comprising up to about 3 (e.g., 1, 2, or 3) amino acid substitutions in any of the HC-CDRs and LC-CDRs; (2) i) an HC-CDR1 comprising the amino acid sequence of SEQ ID NO: 2; ii) an HC-CDR2 comprising the amino acid sequence of SEQ ID NO: 8; iii) an HC-CDR3 comprising the amino acid sequence of SEQ ID NO: 13; iv) an LC-CDR1 comprising the amino acid sequence of SEQ ID NO: 17; v) an LC-CDR2 comprising the amino acid sequence of SEQ ID NO: 20; and vi) an LC-CDR3 comprising the amino acid sequence of SEQ ID NO: 25; or a variant thereof comprising up to about 3 (e.g., 1, 2, or 3) amino acid substitutions in any of the HC-CDRs and LC-CDRs; (3) i) an HC-CDR1 comprising the amino acid sequence of SEQ ID NO: 3; ii) an HC-CDR2 comprising the amino acid sequence of SEQ ID NO: 9; iii) an HC-CDR3 comprising the amino acid sequence of SEQ ID NO: 14; iv) an LC-CDR1 comprising the amino acid sequence of SEQ ID NO: 17; v) an LC-CDR2 comprising the amino acid sequence of SEQ ID NO: 21; and vi) an LC-CDR3 comprising the amino acid sequence of SEQ ID NO: 26; or a variant thereof comprising up to about 3 (e.g., 1, 2, or 3) amino acid substitutions in any of the HC-CDRs and LC-CDRs; (4) i) an HC-CDR1 comprising the amino acid sequence of SEQ ID NO: 4; ii) an HC-CDR2 comprising the amino acid sequence of SEQ ID NO: 7; iii) an HC-CDR3 comprising the amino acid sequence of SEQ ID NO: 12; iv) an LC-CDR1 comprising the amino acid sequence of SEQ ID NO: 17; v) an LC-CDR2 comprising the amino acid sequence of SEQ ID NO: 19; and vi) an LC-CDR3 comprising the amino acid sequence of SEQ ID NO: 24; or a variant thereof comprising up to about 3 (e.g., 1, 2, or 3) amino acid substitutions in any of the HC-CDRs and LC-CDRs; (5) i) an HC-CDR1 comprising the amino acid sequence of SEQ ID NO: 5; ii) an HC-CDR2 comprising the amino acid sequence of SEQ ID NO: 10; iii) an HC-CDR3 comprising the amino acid sequence of SEQ ID NO: 15; iv) an LC-CDR1 comprising the amino acid sequence of SEQ ID NO: 18; v) an LC-CDR2 comprising the amino acid sequence of SEQ ID NO: 22; and vi) an LC-CDR3 comprising the amino acid sequence of SEQ ID NO: 27; or a variant thereof comprising up to about 3 (e.g., 1, 2, or 3) amino acid substitutions in any of the HC-CDRs and LC-CDRs; or (6) i) an HC-CDR1 comprising the amino acid sequence of SEQ ID NO: 6; ii) an HC-CDR2 comprising the amino acid sequence of SEQ ID NO: 11; iii) an HC-CDR3 comprising the amino acid sequence of SEQ ID NO: 16; iv) an LC-CDR1 comprising the amino acid sequence of SEQ ID NO: 17; v) an LC-CDR2 comprising the amino acid sequence of SEQ ID NO: 23; and vi) an LC-CDR3 comprising the amino acid sequence of SEQ ID NO: 28; or a variant thereof comprising up to about 3 (e.g., 1, 2, or 3) amino acid substitutions in any of the HC-CDRs and LC-CDRs. In some embodiments, the multivalent (e.g., bivalent) anti-Ang2 antibody (e.g., full length antibody) or antigen-binding fragment thereof comprises any of the following: (1) i) an HC-CDR1 comprising the amino acid sequence of SEQ ID NO: 1; ii) an HC-CDR2 comprising the amino acid sequence of SEQ ID NO: 7; iii) an HC-CDR3 comprising the amino acid sequence of SEQ ID NO: 12; iv) an LC-CDR1 comprising the amino acid sequence of SEQ ID NO: 17; v) an LC-CDR2 comprising the amino acid sequence of SEQ ID NO: 19; and vi) an LC-CDR3 comprising the amino acid sequence of SEQ ID NO: 24; (2) i) an HC-CDR1 comprising the amino acid sequence of SEQ ID NO: 2; ii) an HC-CDR2 comprising the amino acid sequence of SEQ ID NO: 8; iii) an HC-CDR3 comprising the amino acid sequence of SEQ ID NO: 13; iv) an LC-CDR1 comprising the amino acid sequence of SEQ ID NO: 17; v) an LC-CDR2 comprising the amino acid sequence of SEQ ID NO: 20; and vi) an LC-CDR3 comprising the amino acid sequence of SEQ ID NO: 25; (3) i) an HC-CDR1 comprising the amino acid sequence of SEQ ID NO: 3; ii) an HC-CDR2 comprising the amino acid sequence of SEQ ID NO: 9; iii) an HC-CDR3 comprising the amino acid sequence of SEQ ID NO: 14; iv) an LC-CDR1 comprising the amino acid sequence of SEQ ID NO: 17; v) an LC-CDR2 comprising the amino acid sequence of SEQ ID NO: 21; and vi) an LC-CDR3 comprising the amino acid sequence of SEQ ID NO: 26; (4) i) an HC-CDR1 comprising the amino acid sequence of SEQ ID NO: 4; ii) an HC-CDR2 comprising the amino acid sequence of SEQ ID NO: 7; iii) an HC-CDR3 comprising the amino acid sequence of SEQ ID NO: 12; iv) an LC-CDR1 comprising the amino acid sequence of SEQ ID NO: 17; v) an LC-CDR2 comprising the amino acid sequence of SEQ ID NO: 19; and vi) an LC-CDR3 comprising the amino acid sequence of SEQ ID NO: 24; (5) i) an HC-CDR1 comprising the amino acid sequence of SEQ ID NO: 5; ii) an HC-CDR2 comprising the amino acid sequence of SEQ ID NO: 10; iii) an HC-CDR3 comprising the amino acid sequence of SEQ ID NO: 15; iv) an LC-CDR1 comprising the amino acid sequence of SEQ ID NO: 18; v) an LC-CDR2 comprising the amino acid sequence of SEQ ID NO: 22; and vi) an LC-CDR3 comprising the amino acid sequence of SEQ ID NO: 27; or (6) i) an HC-CDR1 comprising the amino acid sequence of SEQ ID NO: 6; ii) an HC-CDR2 comprising the amino acid sequence of SEQ ID NO: 11; iii) an HC-CDR3 comprising the amino acid sequence of SEQ ID NO: 16; iv) an LC-CDR1 comprising the amino acid sequence of SEQ ID NO: 17; v) an LC-CDR2 comprising the amino acid sequence of SEQ ID NO: 23; and vi) an LC-CDR3 comprising the amino acid sequence of SEQ ID NO: 28.

In some embodiments, the multivalent (e.g., bivalent) anti-Ang2 antibody (e.g., full length antibody) or antigen-binding fragment thereof comprises i) an VH comprising the amino acid sequence of any of SEQ ID NOs: 82, 83, 87, 89, 90, 93, 94, and 96, or a variant thereof having at least about 85% sequence identify to any of SEQ ID NOs: 82, 83, 87, 89, 90, 93, 94, and 96; and ii) an VL comprising the amino acid sequence of any of SEQ ID NOs: 84-86, 88, 91, 92, 95, 97, and 98, or a variant thereof having at least about 85% sequence identify to any one of SEQ ID NOs: 84-86, 88, 91, 92, 95, 97, and 98. In some embodiments, the multivalent (e.g., bivalent) anti-Ang2 antibody (e.g., full length antibody) or antigen-binding fragment thereof comprises any of the following: (1) a VH comprising the amino acid sequence of SEQ ID NO: 82 or 83, and an VL comprising the amino acid sequence of any of SEQ ID NOs: 84-86; (2) a VH comprising the amino acid sequence of SEQ ID NO: 87, and an VL comprising the amino acid sequence of SEQ ID NO: 88 or 98; (3) a VH comprising the amino acid sequence of SEQ ID NO: 89 or 90, and an VL comprising the amino acid sequence of SEQ ID NO: 91 or 92; (4) a VH comprising the amino acid sequence of SEQ ID NO: 93, and an VL comprising the amino acid sequence of any of SEQ ID NOs: 84-86; (5) a VH comprising the amino acid sequence of SEQ ID NO: 94, and an VL comprising the amino acid sequence of SEQ ID NO: 95; or (6) a VH comprising the amino acid sequence of SEQ ID NO: 96, and an VL comprising the amino acid sequence of SEQ ID NO: 97. In some embodiments, the multivalent (e.g., bivalent) anti-Ang2 antibody (e.g., full length antibody) or antigen-binding fragment thereof comprises any of the following: (1) a VH comprising the amino acid sequence of SEQ ID NO: 82, and an VL comprising the amino acid sequence of SEQ ID NO: 85; (2) a VH comprising the amino acid sequence of SEQ ID NO: 83, and an VL comprising the amino acid sequence of any of SEQ ID NOs: 84-86; (3) a VH comprising the amino acid sequence of SEQ ID NO: 87, and an VL comprising the amino acid sequence of SEQ ID NO: 88; (4) a VH comprising the amino acid sequence of SEQ ID NO: 89, and an VL comprising the amino acid sequence of SEQ ID NO: 92; (5) a VH comprising the amino acid sequence of SEQ ID NO: 90, and an VL comprising the amino acid sequence of SEQ ID NO: 91; (6) a VH comprising the amino acid sequence of SEQ ID NO: 93, and an VL comprising the amino acid sequence of SEQ ID NO: 85; (7) a VH comprising the amino acid sequence of SEQ ID NO: 94, and an VL comprising the amino acid sequence of SEQ ID NO: 95; or (8) a VH comprising the amino acid sequence of SEQ ID NO: 96, and an VL comprising the amino acid sequence of SEQ ID NO: 97.

In some embodiments, the multivalent anti-Ang2 antibody or antigen-binding fragment thereof is an anti-Ang2 full length antibody. In some embodiments, the anti-Ang2 full length antibody comprises i) a heavy chain comprising the amino acid sequence of any of SEQ ID NOs: 33-40; and ii) a light chain comprising the amino acid sequence of any of SEQ ID NOs: 41-49. In some embodiments, the multivalent (e.g., bivalent) anti-Ang2 antibody (e.g., full length antibody) or antigen-binding fragment thereof comprises any of the following: (1) a heavy chain comprising the amino acid sequence of SEQ ID NO: 33 or 34, and a light chain comprising the amino acid sequence of any of SEQ ID NOs: 41-43; (2) a heavy chain comprising the amino acid sequence of SEQ ID NO: 35, and a light chain comprising the amino acid sequence of SEQ ID NO: 44 or 49; (3) a heavy chain comprising the amino acid sequence of SEQ ID NO: 36 or 37, and a light chain comprising the amino acid sequence of SEQ ID NO: 45 or 46; (4) a heavy chain comprising the amino acid sequence of SEQ ID NO: 38, and a light chain comprising the amino acid sequence of any of SEQ ID NOs: 41-43; (5) a heavy chain comprising the amino acid sequence of SEQ ID NO: 39, and a light chain comprising the amino acid sequence of SEQ ID NO: 47; or (6) a heavy chain comprising the amino acid sequence of SEQ ID NO: 40, and a light chain comprising the amino acid sequence of SEQ ID NO: 48. In some embodiments, the anti-Ang2 full length antibody comprises any of the following: (1) a heavy chain comprising the amino acid sequence of SEQ ID NO: 33, and a light chain comprising the amino acid sequence of SEQ ID NO: 42; (2) a heavy chain comprising the amino acid sequence of SEQ ID NO: 34, and a light chain comprising the amino acid sequence of any of SEQ ID NOs: 41-43; (3) a heavy chain comprising the amino acid sequence of SEQ ID NO: 35, and a light chain comprising the amino acid sequence of SEQ ID NO: 44; (4) a heavy chain comprising the amino acid sequence of SEQ ID NO: 36, and a light chain comprising the amino acid sequence of SEQ ID NO: 46; (5) a heavy chain comprising the amino acid sequence of SEQ ID NO: 37, and a light chain comprising the amino acid sequence of SEQ ID NO: 45; (6) a heavy chain comprising the amino acid sequence of SEQ ID NO: 38, and a light chain comprising the amino acid sequence of SEQ ID NO: 42; (7) a heavy chain comprising the amino acid sequence of SEQ ID NO: 39, and a light chain comprising the amino acid sequence of SEQ ID NO: 47; or (8) a heavy chain comprising the amino acid sequence of SEQ ID NO: 40, and a light chain comprising the amino acid sequence of SEQ ID NO: 48. In some embodiments, the anti-Ang2 full length antibody is monospecific. In some embodiments, the anti-Ang2 full length antibody is bispecific.

### Fc Domain

In some embodiments, the antibody fusion protein descried herein or the multivalent (e.g., bivalent) anti-Ang2 antibody (e.g., full length antibody) or antigen-binding fragment thereof comprises an Fc domain or portion thereof. Fc domain comprises a CH2 domain and a CH3 domain. In some embodiments, the Fc domain portion comprises (consists essentially of or consists of) a CH2 domain. In some embodiments, the Fc domain portion comprises (consists essentially of or consists of) a CH3 domain.

In some embodiments, the Fc domain is derived from any of IgA, IgD, IgE, IgG, and IgM, and subtypes thereof. In some embodiments, the Fc domain is derived from an IgG (e.g., IgG1, IgG2, IgG3, or IgG4). In some embodiments, the Fc domain is derived from a human IgG. In some embodiments, the Fc domain is derived from a human IgG1 or human IgG4. In some embodiments, the two subunits of the Fc domain dimerize via one or more (e.g., 1, 2, 3, 4, or more) disulfide bonds. In some embodiments, each subunit of the Fc domain comprises a full-length Fc sequence. In some embodiments, each subunit of the Fc domain comprises an N-terminus truncated Fc sequence. In some embodiments, the Fc domain is truncated at the N-terminus, e.g., lacks the first 1, 2, 3, 4, 5, 6, 7, 8, 9, or 10 amino acids of a complete immunoglobulin Fc domain.

Via the Fc domain, the antibody fusion protein descried herein or the multivalent (e.g., bivalent) anti-Ang2 antibody (e.g., full length antibody) or antigen-binding fragment thereof can activate complement and interact with Fc receptors (FcRs). In some embodiments, this inherent immunoglobulin feature is viewed unfavorably because antibody fusion protein may be targeted to cells expressing FcRs rather than the preferred antigen-bearing cells or target molecule expressing tissues. Moreover, the simultaneous activation of Tie2 and FcR signaling pathways may over-stimulate cytokine release, especially in combination with the long half-life of immunoglobulin fusion proteins, make their application in a therapeutic setting difficult due to systemic toxicity. Although the presence of an Fc domain is essential for prolonging the half-life of the anti-Ang2/VEGF-trap antibody fusion protein, in some situations it will be beneficial to eliminate effector functions associated with engagement of FcRs by the Fc domain. Thus in some embodiments, the Fc domain is engineered to have altered binding to an FcR, specifically altered binding to an Fcγ receptor, and/or altered effector function, such as altered (e.g., reduced or eliminated) ADCC, ADCP, and/or CDC. In some embodiments the altered binding to an FcR and/or effector function is reduced binding and/or effector function. In some embodiments, the Fc domain comprises one or more amino acid mutation that reduces the binding of the Fc domain to an FcR, particularly an Fcγ receptor (responsible for ADCC). Preferably, such an amino acid mutation does not reduce binding to FcRn receptors (responsible for half-life). In some embodiments, the Fc domain is derived from human IgG1 and comprises the amino acid substitution N297A. In some embodiments, the Fc domain is derived from human IgG4 and comprises the amino acid substitutions S228P and/or L235E at the hinge region. In some embodiments, the Fc domain is derived from human IgG4 and comprises the amino acid substitutions S228P at the hinge region. In some embodiments, the Fc domain is derived from human IgG1 and comprises the amino acid substitutions L234A and L235A (“LALA”) at the hinge region. In some embodiments, the Fc domain is derived from human IgG1 and comprises the amino acid substitutions L234A and L235A at the hinge region, and P329G, e.g., in each of its subunits. See, e.g., Lo M. et al. J Biol Chem. 2017; 292(9):3900-3908; Schlothauer T. et al. Protein Eng Des Sel. 2016; 29(10):457-466.

In some embodiments, the Fc domain (e.g., human IgG1) is mutated to remove one or more effector functions such as ADCC, ADCP, or CDC, namely, an “effectorless” or “almost effectorless” Fc domain. For example, in some embodiments, the Fc domain is an effectorless IgG1 Fc comprising one or more of the following mutations (such as in each of its subunits): L234A, L235E, G237A, A330S, and P331S. In some embodiments, effector function is eliminated through a mutation in the constant region that eliminated glycosylation, e.g., “effectorless mutation.” In some embodiments, the effectorless mutation is an N297A or DANA mutation (D265A+N297A) in the C_{H}2 region. Shields et al., J. Biol. Chem. 276 (9): 6591-6604 (2001). The combinations of K322A, L234A, and L235A in IgG1 are sufficient to almost completely abolish FcγR and C1q binding (Hezareh et al. J Virol 75, 12161-12168, 2001). Medlmmune identified that a set of three mutations L234F/L235E/P331S have a very similar effect (Oganesyan et al., Acta Crystallographica 64, 700-704, 2008). In some embodiments, the Fc domain comprises a modification of the glycosylation on N297 of the IgG1 Fc domain, which is known to be required for optimal FcR interaction. The Fc domain modification can be any suitable IgG Fc engineering mentioned in Wang et al. (“IgG Fc engineering to modulate antibody effector functions,” Protein Cell. 2018 January; 9(1): 63-73), the content of which is incorporated herein by reference in its entirety. Alternatively, effector function can be reduced or eliminated through production techniques, such as expression in host cells that do not glycosylate (e.g., E. coli) or in which result in an altered glycosylation pattern that is ineffective or less effective at promoting effector function (e.g., Shinkawa et al., J. Biol. Chem. 278(5): 3466-3473 (2003).

In some embodiments, the Fc domain comprises two identical polypeptide chains (identical Fc subunits). Such Fc domains are herein also referred to as “homodimeric Fc domains.”

In some embodiments, the Fc domain comprises a modification promoting heterodimerization of two non-identical polypeptide chains. Such Fc domains are herein also referred to as “heterodimeric Fc domains.” In some embodiments, the Fc domain comprises a knob-into-hole (KIH) modification, comprising a knob modification in one of the subunits of the Fc domain and a hole modification in the other one of the two subunits of the Fc domain. Any suitable knob-into-hole modifications can be applied to the multivalent (e.g., bivalent) anti-Ang2 antibodies or antigen-binding fragments thereof (or anti-Ang2/VEGF-trap antibody fusion protein) described herein, such as amino acid changes of T22>Y (creating the knob) in strand B of the first CH3 domain and Y86>T (creating the hole) in strand E of the partner CH3 domain. Also see US20200087414, the content of which is incorporated herein by reference in its entirety. In some embodiments, one subunit of the Fc domain comprises Y349C, T366S, L368A, and Y407V mutations relative to a wildtype human IgG1 Fc, and the other subunit of the Fc domain comprises S354C and T366W mutations relative to a wildtype human IgG1 Fc.

### Antibody or Antibody Fusion Protein Variants

In some embodiments, amino acid sequence variants of the multivalent (e.g., bivalent) anti-Ang2 antibodies or antigen-binding fragments thereof (or anti-Ang2/VEGF-trap antibody fusion protein) described herein are contemplated, hereinafter also referred to as “antibody or antibody fusion protein variants”. For example, it may be desirable to improve the binding affinity and/or other biological properties of the antibody fusion protein. Amino acid sequence variants of a construct may be prepared by introducing appropriate modifications into the nucleic acid sequence encoding the construct, or by peptide synthesis. Such modifications include, for example, deletions from, and/or insertions into and/or substitutions of residues within the amino acid sequences of the construct. Any combination of deletion, insertion, and substitution can be made to arrive at the final construct, provided that the final construct possesses the desired characteristics, e.g., Ang2-binding and Tie2 activation.

### a) Substitution, Insertion, Deletion and Variants

Sites of interest for mutagenesis (e.g., substitutions, insertions, or deletions) include the HVRs and FRs of an antibody. Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. In some embodiments, substitutions, insertions, or deletions may occur within one or more HVRs so long as such alterations do not substantially reduce the ability of the antibody or antigen-binding fragment thereof (or antibody fusion protein) to bind antigen. Conservative substitutions are shown in Table B. More substantial changes are provided under the heading of “exemplary substitutions.” Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class.

TABLE B
Amino acid substitutions

| Original | Exemplary | Preferred | Original | Exemplary | Preferred |
|---|---|---|---|---|---|
| Ala (A) | Val; Leu; Ile | Val | Leu (L) | Norleucine; Ile; Val; | Ile |
| | | | | Met; Ala; Phe | |
| Arg (R) | Lys; Gln; Asn | Lys | Lys (K) | Arg; Gln; Asn | Arg |
| Asn (N) | Gln; His; Asp, Lys; | Gln | Met (M) | Leu; Phe; Ile | Leu |
| | Arg | | | | |
| Asp (D) | Glu; Asn | Glu | Phe (F) | Trp; Leu; Val; Ile; | Tyr |
| | | | | Ala; Tyr | |
| Cys (C) | Ser; Ala | Ser | Pro (P) | Ala | Ala |
| Gln (Q) | Asn; Glu | Asn | Ser (S) | Thr | Thr |
| Glu (E) | Asp; Gln | Asp | Thr (T) | Val; Ser | Ser |
| Gly (G) | Ala | Ala | Trp (W) | Tyr; Phe | Tyr |
| His (H) | Asn; Gln; Lys; Arg | Arg | Tyr (Y) | Trp; Phe; Thr; Ser | Phe |
| Ile (I) | Leu; Val; Met; Ala; | Leu | Val (V) | Ile; Leu; Met; Phe; | Leu |
| | Phe; Norleucine | | | Ala; Norleucine | |

A useful method for identification of residues or regions of an antibody that may be targeted for mutagenesis is called “alanine scanning mutagenesis” as described by Cunningham and Wells (1989) Science, 244:1081-1085. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

### b) Glycosylation Variants

In some embodiments, the multivalent anti-Ang2 antibody or antigen-binding fragment thereof (or anti-Ang2/VEGF-trap antibody fusion protein) is altered to increase or decrease the extent to which the construct is glycosylated. Addition or deletion of glycosylation sites to an Fc domain may be conveniently accomplished by altering the amino acid sequence such that one or more glycosylation sites is created or removed.

Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an Fc domain may be made in order to create certain improved properties.

In some embodiments, the multivalent anti-Ang2 antibody or antigen-binding fragment thereof (or antibody fusion protein) is provided having a carbohydrate structure that lacks fucose attached (directly or indirectly) to the Fc domain. For example, the amount of fucose in such multivalent anti-Ang2 antibody or antigen-binding fragment thereof (or antibody fusion protein) may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc domain (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US 2003/0157108; US 2004/0093621; etc. Examples of publications related to “defucosylated” or “fucose-deficient” antibody variants include: US 2003/0157108; WO2005/053742; Okazaki et al. J. Mol. Biol. 336:1239-1249 (2004); Yamane-Ohnuki et al. Biotech. Bioeng. 87: 614 (2004); etc. Examples of cell lines capable of producing defucosylated antibodies include Lec13 CHO cells deficient in protein fucosylation (e.g., WO 2004/056312 A1, Adams et al., especially at Example 11), and knockout cell lines, such as alpha-1,6-fucosyltransferase gene, FUT8, knockout CHO cells (see, e.g., Kanda, Y. et al., Biotechnol. Bioeng., 94(4):680-688 (2006); or WO2003/085107).

### c) Fc Domain Variants

In some embodiments, the present application contemplates an anti-Ang2/VEGF-trap antibody fusion protein that possesses some but not all Fc effector functions, which makes it a desirable candidate for applications in which the half-life of the antibody fusion protein in vivo is important yet certain effector functions (such as CDC and ADCC) are unnecessary or deleterious. Some of the Fc domain variants have been discussed above. In vitro and/or in vivo cytotoxicity assays can be conducted to confirm the reduction/depletion of CDC and/or ADCC activities. For example, FcR binding assays can be conducted to ensure that the antibody or antibody fusion protein lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96 ® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). Clq binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Fc domains with reduced effector function include those with substitution of one or more of Fc region residues 238, 265, 269, 270, 297, 327 and 329 (U.S. Pat. No. 6,737,056). Such Fc mutants include substitutions at two or more of amino acid positions 265, 269, 270, 297 and 327, including the so-called “DANA” Fc mutant with substitution of residues 265 and 297 to alanine (U.S. Pat. No. 7,332,581). Certain antibody variants with improved or diminished binding to FcRs are described (see, e.g., U.S. Pat. No. 6,737,056; WO 2004/056312, and Shields et al., J. Biol. Chem. 9(2): 6591-6604 (2001)). In some embodiments, alterations are made in the Fc domain that result in altered (i.e., either improved or diminished) C1q binding and/or CDC, e.g., as described in U.S. Pat. No. 6,194,551, WO 99/51642, and Idusogie et al. J. Immunol. 164: 4178-4184 (2000).

In some embodiments, the Fc domain comprises one or more amino acid substitutions, which increase half-life and/or improve binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc domain with one or more substitutions therein which improve binding of the Fc region to FcRn. Such Fc variants include those with substitutions at one or more of Fc region residues, e.g., substitution of Fc region residue 434 (U.S. Pat. No. 7,371,826).

### d) Cysteine Engineered Variants

In some embodiments, it may be desirable to create cysteine-engineered multivalent anti-Ang2 antibodies or antigen-binding fragments thereof (or anti-Ang2/VEGF-trap antibody fusion proteins), e.g., “thioMAbs,” in which one or more residues of an antibody or antigen-binding fragment thereof (or antibody fusion protein) are substituted with cysteine residues. In particular embodiments, the substituted residues occur at accessible sites of the antibody or antigen-binding fragment thereof (or antibody fusion protein). By substituting those residues with cysteine, reactive thiol groups are thereby positioned at accessible sites of the antibody or antigen-binding fragment thereof (or antibody fusion protein) and may be used to conjugate the antibody or antigen-binding fragment thereof (or antibody fusion protein) to other moieties, such as drug moieties or linker-drug moieties, to create a conjugate (e.g., antibody fusion protein-conjugate). In some embodiments, any one or more of the following residues may be substituted with cysteine: A118 (EU numbering) of the heavy chain; and 5400 (EU numbering) of the heavy chain Fc domain. Cysteine engineered antibody/antigen-binding fragment thereof (or antibody fusion protein) may be generated as described, e.g., in U.S. Pat. No. 7,521,541.

### e) Antibody or Antibody Fusion Protein Derivatives

In some embodiments, multivalent anti-Ang2 antibodies or antigen-binding fragments thereof (or anti-Ang2/VEGF-trap antibody fusion proteins) provided herein may further comprise an additional therapeutic compound, such as any therapeutic compounds known in the art. For example, the parental antibody in some embodiments can be an antibody drug conjugate (ADC). See, e.g., any ADC described in Shim H. (Biomolecules. 2020 March; 10(3): 360), and Diamantis N. and Banerji U. Br J Cancer. 2016; 114(4): 362-367, the contents of which are incorporated herein by reference in their entirety. In some embodiments, the therapeutic compound is conjugated to the Fc domain or portion thereof. In some embodiments, the therapeutic compound is a cytotoxic agent, a chemotherapeutic agent, a growth inhibitory agent, or an antibiotic.

In some embodiments, the multivalent anti-Ang2 antibody or antigen-binding fragment thereof (or anti-Ang2/VEGF-trap antibody fusion protein) further comprises a label selected from the group consisting of a chromophore, a fluorophore (e.g., coumarin, a xanthene, a cyanine, a pyrene, a borapolyazaindacene, an oxazine, and derivatives thereof), a fluorescent protein (e.g., GFP, phycobiliproteins, and derivatives thereof), a phosphorescent dye (e.g., dioxetanes, xanthene, or carbocyanine dyes, lanthanide chelates), a tandem dye (e.g., cyanine-phycobiliprotein derivative and xanthene-phycobiliprotein derivative), a particle (e.g., gold clusters, colloidal gold, microspheres, quantum dots), a hapten, an enzyme (e.g., peroxidase, a phosphatase, a glycosidase, a luciferase), and a radioisotope (e.g., ¹²⁵I, ³H, ¹⁴C, ³²P).

### VEGFR Component

In mammals, the VEGF family comprises five members: VEGF-A, VEGF-B, VEGF-C, VEGF-D, and placental growth factor (PlGF). In addition, multiple isoforms of VEGF-A, VEGF-B, and PlGF are generated through alternative RNA splicing (Sullivan et al., MAbs, 2002, 2(2): 165-75). All VEGF family members bind as homodimers to tyrosine kinase receptors (TKRs; the VEGFRs) on the cell surface, causing them to dimerize and become activated through transphosphorylation. The VEGFRs have an extracellular portion consisting of 7 immunoglobulin (Ig)-like domains, a single transmembrane spanning region and an intracellular portion containing a split tyrosine-kinase domain. VEGF-A is the primary factor involved with angiogenesis. It binds to both VEGFR-1 (Flt-1) and VEGFR-2 (KDR/Flk-1), two high affinity tyrosine kinase receptors (TKRs) expressed almost exclusively on endothelial cells. VEGF-B binds selectively to VEGFR-1 (Flt-1). VEGF-C and VEGF-D, but not VEGF-A, are ligands for VEGFR-3 (Flt4), which mediates lymphangiogenesis. VEGF-C can stimulate lymphangiogenesis (via VEGFR3) and angiogenesis via VEGFR2.

In some embodiments, the VEGFR component of the present invention comprises one or more Ig-like domains derived from one or more of Flt1, Flk1, or Flt4 extracellular ligand-binding region, which mediates binding to the VEGF proteins. In some embodiments, the VEGFR component comprises Ig-like domains derived from extracellular ligand-binding regions of both Flt1 and Flk1, or both Flt1 and Flt4, or functional equivalents thereof, forming a “chimeric VEGFR” component.

The term “Ig-like domain” of Flt-1, Flt-4, or Flk-1 is intended to encompass not only the complete wild-type domain, but also insertional, deletional, and/or substitutional variants thereof which substantially retain the functional characteristics of the intact domain. It will be readily apparent to one of skill in the art that numerous variants of the Ig-like domains can be obtained which will retain substantially the same functional characteristics as the wild-type domain.

The term “functional equivalents” when used in reference to an Ig-like domain “X”, is intended to encompass the Ig-like domain “X” with at least one alteration, e.g., a deletion, addition, and/or substitution, which retains substantially the same functional characteristics as does the wild type Ig-like domain “X”, that is, a substantially equivalent binding to VEGF. It will be appreciated that various amino acid substitutions can be made in an Ig-like domain “X” without departing from the spirit of the invention with respect to the ability of these receptor components to bind and inactivate/neutralize VEGF. Thus, point mutational and other broader variations may be made in the Ig-like domain or domains of the VEGFR component of the present invention so as to impart interesting properties that do not substantially affect the VEGFR component's ability to bind to and inhibit the activity of VEGF. The functional characteristics of the VEGFR components may be determined by any suitable screening assay known to the art for measuring the desired characteristic. Other assays, for example, a change in the ability to specifically bind to VEGF can be measured by a competition-type VEGF binding assay. Modifications of protein properties such as thermal stability, hydrophobicity, susceptibility to proteolytic degradation, or tendency to aggregate may be measured by methods known to those of skill in the art. Also see subsection “1. Amino acid sequence variants” under section “V. Methods of preparation.”

In some embodiments, the VEGFR component comprises one or more (e.g., 1, 2, 3, 4, 5, 6, 7, or more) Ig-like domains of one or more (e.g., 1, 2, 3, 4, 5, or more) VEGFRs independently selected from the group consisting of Flt1, Flk1, and Flt4. In some embodiments, the VEGFR component comprises an Ig-like domain of either Flt-1 or Flk-1. In some embodiments, the VEGFR component comprises one or more of an Ig-like domain 1 of Flt-1 (hereinafter referred to as Flt1d1), an Flt1d2, an Flt1d3, an Fin d4, an Flt1d5, an Flt1d6, or an Flt1d7. In some embodiments, the VEGFR component comprises one or more of an Ig-like domain 1 of Flk-1 (hereinafter referred to as Flk1d1), an Flk1d2, an Flk1d3, an Flk1d4, an Flk1d5, an Flk1d6, or an Flk1d7. In some embodiments, the VEGFR component comprises one or more of an Ig-like domain 1 of Flt-4 (hereinafter referred to as Flt4d1), an Flt4d2, an Flt4d3, an Flt4d4, an Flt4d5, an Flt4d6, or an Flt4d7. In some embodiments, the VEGFR component comprises an Flt1d2. In some embodiments, the VEGFR component comprises an Flk1d3. In some embodiments, the VEGFR component comprises an Ig-like domain 2 of a first VEGFR and an Ig-like domain 3 of a second VEGFR. In some embodiments, the first VEGFR is Flt1, and the second VEGFR is Flk1 or Flt4.

In some embodiments, the VEGFR component comprises 1, 2, 3, 4, 5, 6, or 7 Ig-like domains of Flt1, Flk1, or Flt4. In some embodiments, the VEGFR component comprises 2, 3, 4, 5, 6, or 7 Ig-like domains, comprising a mixture of Ig-like domains from Flt-1 and Flk-1, or Flt1 and Flt4. For example, in some embodiments, the VEGFR component comprises one Ig-like domain from Flk-1, and two Ig-like domains from Flt-1. In some embodiments, the VEGFR component comprises 2, 3, 4, 5, 6, or 7 Ig-like domains all from Flt-1. In some embodiments, the VEGFR component comprises 2, 3, 4, 5, 6, or 7 Ig-like domains all from Flk-1. In some embodiments, the VEGFR component comprises 2, 3, 4, 5, 6, or 7 Ig-like domains all from Flt4. In some embodiments, the VEGFR component consists of 1, 2, 3, 4, 5, 6, or 7 Ig-like domains of Flt1, Flk1, and/or Flt4. In some embodiments, the VEGFR component consists essentially of 1, 2, 3, 4, 5, 6, or 7 Ig-like domains of Flt1, Flk1, and/or Flt4. In some embodiments, the VEGFR component comprises a moiety that is not Ig-like domains from Flt1, Flk1, or Flt4.

In some embodiments, the two or more Ig-like domains within a VEGFR component are fused directly to each other. In some embodiments, the two or more Ig-like domains within a VEGFR component are fused to each other via one or more domain linkers, such as the endogenous domain linkers from Flt1, Flk1, or Flt4, e.g., the domain linker that connects Flt1d2 and Flt1d3. In some embodiments, the Ig-like domains within the VEGFR component are connected by peptide linkers (see, e.g. “peptide linkers” subsection below), such as (GGGGS), (SEQ ID NO: 106), wherein n is an integer of at least 1, e.g., (GGGGS) 3 (SEQ ID NO: 30). In some embodiments, the Ig-like domains within the VEGFR component are connected by non-peptide linkers (see, e.g. “non-peptide linkers” subsection below), such as thioesters. The linker sequence may be provided to decrease steric hindrance such that the one or more VEGFR components and the multivalent anti-Ang2 antibody or antigen-binding fragment thereof may assume their optimal tertiary structure and/or interact appropriately with their corresponding target molecule.

In some embodiments, the VEGFR component comprises an Flt1d2. In some embodiments, the VEGFR component further comprises an Flk1d3. In some embodiments, the VEGFR component further comprises an Ig-like domain of a third VEGFR, and optionally an Ig-like domain of a fourth VEGFR, wherein the third/fourth Ig-like domain is selected from the group consisting of Flk1d1, Flk1d4, Flk1d5, Flt1d4, and Flt1d5. In some embodiments, the VEGFR component further comprises an Flk1d4. In some embodiments, the VEGFR component comprises Flt1d2 and Flk1d3. In some embodiments, the VEGFR component consists essentially of Flt1d2 and Flk1d3. In some embodiments, the VEGFR component consists of Flt1d2 and Flk1d3. In some embodiments, the VEGFR component comprises (or consists essentially of, or consists of) an amino acid sequence of SEQ ID NO: 32. In some embodiments, the VEGFR component comprises Flt1d2, Flk1d3, and Flk1d4. In some embodiments, the VEGFR component consists essentially of Flt1d2, Flk1d3, and Flk1d4. In some embodiments, the VEGFR component consists of Fin d2, Flk1d3, and Flk1d4.

The VEGFR component can have Ig-like domains of any arrangements. For example, in some embodiments, the antibody fusion protein comprising a VEGFR component fused to the C-terminus of a light chain of an anti-Ang2 full length antibody comprises (or consists of or consists essentially of) a light chain-VEGFR component fusion polypeptide of any of the following configurations (from N-terminus to C-terminus), wherein L is an optional linker (e.g., peptide linker): (1) VL-CL-L-Flk1d3-Flt1d2; (2) VL-CL-L-Flt1d2-Flk1d3; (3) VL-CL-L-Flk1d4-Flk1d3-Flt1d2; or (4) VL-CL-L-Flt1d2-Flk1d3-Flk1d4, etc. In some embodiments, the antibody fusion protein comprises two or more VEGFR components, which can be the same or different (e.g., comprising different Ig-like domains, different linkers between Ig-like domains, different numbers of Ig-like domains, and/or different arrangement of the Ig-like domains). For example, in some embodiments, the antibody fusion protein comprising a first VEGFR component fused to the C-terminus of a first light chain of an anti-Ang2 full length antibody, and a second VEGFR component fused to the C-terminus of a second light chain of the anti-Ang2 full length antibody, wherein the two light chain fusion polypeptides can comprise (or consist of or consist essentially of) any of the following configurations (from N-terminus to C-terminus), wherein L is an optional linker (e.g., peptide linker): (1) i) VL-CL-L-Flk1d3-Flt1d2 and ii) VL-CL-L-Flk1d3-Flt1d2; (2) i) VL-CL-L-Flk1d3-Flt1d2 and ii) VL-CL-L-Flt1d2-Flk1d3; (3) i) VL-CL-L-Flt1d2-Flk1d3 and ii) VL-CL-L-Flt1d2-Flk1d3; (4) i) VL-CL-L-Flk1d4-Flk1d3-Flt1d2 and ii) VL-CL-L-Flk1d4-Flk1d3-Flt1d2; (5) i) VL-CL-L-Flk1d4-Flk1d3-Flt1d2 and ii) VL-CL-L-Flk1 d3-Flt1d2; (6) i) VL-CL-L-Flt1d2-Flk1d3-Flk1 d4 and ii) VL-CL-L-Flt1d2-Flk1d3-Flk1d4; (7) i) VL-CL-L-Flt1d2-Flk1d3 and ii) VL-CL-L-Flt1d2-Flk1d3-Flk1d4; (8) i) VL-CL-L-Flk1d3-Flk1d4 and ii) VL-CL-L-Flt1d2-Flk1d3-Flk1 d4; (9) i) VL-CL-L-Flk1d4-Flk1d3-Flt1 d2 and ii) VL-CL-L-Flk1d4-Flk1d3; etc.

In some embodiments, the Ig-like domain(s) of Flt1, Flk1, and/or Flt4 are the only Ig-like domain(s) of the VEGFR component. In some embodiments, Flt1d2 is the only Ig-like domain of the VEGFR component. In some embodiments, Flt1d2 and Flk1d3 are the only Ig-like domains of the VEGFR component. In some embodiments, Flt1d2, Flk1d3, and Flk1d4 are the only Ig-like domains of the VEGFR component. In some embodiments, Flk1d1, Flt1d2, and Flk1d3 are the only Ig-like domains of the VEGFR component. In some embodiments, Flt1d2, Flk1d3, and Flt1d4 are the only Ig-like domains of the VEGFR component. In some embodiments, Flt1d2, Flk1d3, and Fin d4 are the only Ig-like domains of the VEGFR component. In some embodiments, Flt1d2, Flk1d3, Flk1d4, and Flk1d5 are the only Ig-like domains of the VEGFR component. In some embodiments, Flt1d2, Flk1d3, Flt1d4, and Flt1d5 are the only Ig-like domains of the VEGFR component.

In some embodiments, the VEGFR component is at least about 4 kDa. In some embodiments, the VEGFR component is about 4 kDa to about 100 kDa, such as any of about 4 kDa to about 15 kDa, about 10 kDa to about 30 kDa, about 10 kDa to about 70 kDa, 10 kDa to about 60 kDa, about 10 kDa to about 50 kDa, 10 kDa to about 40 kDa, about 15 kDa to about 30 kDa, about 20 kDa to about 35 kDa, about 25 kDa to about 40 kDa, about 30 kDa to about 45 kDa, about 35 kDa to about 50 kDa, about 20 kDa to about 30 kDa, about 20 kDa to about 40 kDa, or about 20 kDa to about 50 kDa. In some embodiments, the VEGFR component is about 20 kDa to about 35 kDa, such as about 22 kDa to about 24 kDa.

The VEGFR component is at least about 36 amino acid (aa) in length. In some embodiments, the VEGFR component is about 36 aa to about 900 aa, such as any of about 36 aa to about 100 aa, about 50 aa to about 200 aa, about 100 aa to about 750 aa, about 100 aa to about 650 aa, about 100 aa to about 550 aa, about 100 aa to about 450 aa, about 100 aa to about 350 aa, about 100 aa to about 250 aa, about 150 aa to about 300 aa, about 200 aa to about 350 aa, about 250 aa to about 400 aa, about 300 aa to about 450 aa, about 350 aa to about 500 aa, about 400 aa to about 550 aa, about 450 aa to about 600 aa, about 500 aa to about 650 aa, about 150 aa to about 250 aa, about 150 aa to about 400 aa, about 150 aa to about 500 aa, or about 150 aa to about 600 aa in length. In some embodiments, the VEGFR component is. In some embodiments, the VEGFR component is about 100 aa to about 250 aa, such as about 200 aa to about 250 aa (e.g., 205 aa) in length.

### Activities of the VEGFR Component

In some embodiments, the antibody fusion protein comprises two or more VEGFR components. In some embodiments, the two or more VEGFR components have the same or similar (e.g., within about 2-fold difference) binding affinities. In some embodiments, the two or more VEGFR components have different binding affinities.

Binding specificity and/or affinity of a VEGFR component (such as VEGFR component of the antibody fusion protein) to VEGF can be determined experimentally by methods known in the art. Such methods comprise, but are not limited to Western blots, ELISA, MSD electrochemiluminescence, bead based MIA, RIA, SPR, ECL, IRMA, EIA, Biacore assay, Octet analysis, peptide scans, FACS, or competition-type VEGF binding assays, etc.

For example, in an ELISA-based binding, an ELISA plate can be coated with VEGF, then varying concentrations of VEGFRs or anti-Ang2/VEGF-trap antibody fusion proteins described herein are added into each well. After incubation and washing, a secondary antibody such as HRP conjugated antibody (e.g., anti-IgG Fc for detecting anti-Ang2/VEGF-trap antibody fusion protein comprising an anti-Ang2 full length antibody) can be added in to detect VEGFRs or anti-Ang2/VEGF-trap antibody fusion proteins bound to VEGF. HRP substrate is added to each well. Optical density (OD) of each well can be measured using a microplate reader at 450 nm. EC₅₀ can then be calculated.

In some embodiments, the K_{D} of the binding between the VEGFR component and VEGF is about 10⁻⁵ M to about 10⁻¹⁵ M, such as about 10⁻⁷ M to about 10⁻¹⁵ M, or about 10⁻⁹ M to about 10⁻¹⁵ M.

In some embodiments, fusing the VEGFR component(s) to the multivalent anti-Ang2 antibody or antigen-binding fragment thereof does not affect the binding affinity and/or specificity of the VEGFR component(s) to VEGF, for example, the binding (e.g., K_{D}) between the fused VEGFR component(s) and VEGF is the same as, or within about 2-fold difference of, the binding (e.g., K_{D}) between a corresponding non-fused VEGFR component (e.g., soluble VEGFR) and VEGF. In some embodiments, fusing the VEGFR component(s) to the multivalent anti-Ang2 antibody or antigen-binding fragment thereof reduces the binding affinity and/or specificity of the VEGFR component(s) to VEGF, for example, the K_{D} of the binding between the fused VEGFR component(s) and VEGF is more than about 2 times of the K_{D} of the binding between a corresponding non-fused VEGFR component and VEGF. In some embodiments, fusing the VEGFR component(s) to the multivalent anti-Ang2 antibody or antigen-binding fragment thereof increases the binding affinity and/or specificity of the VEGFR component(s) to VEGF, for example, the K_{D} of the binding between a corresponding non-fused VEGFR component and VEGF is more than about 2 times of the K_{D} of the binding between the fused VEGFR component(s) and VEGF.

The biological activity of a VEGFR component (such as VEGFR component of the antibody fusion protein) can be determined by any methods known in the art, such as competition-type VEGF binding assay, VEGF-dependent receptor phosphorylation assay (e.g., using primary human endothelial cells (HUVECs)), or VEGF-dependent reporter gene expression assay (e.g., using a cell that expresses VEGFR on the cell surface and carries a reporter gene under the control of VEGFR activation), to measure the ability of the VEGFR component (or anti-Ang2/VEGF-trap antibody fusion protein) in blocking/reducing VEGF from binding to and/or activating VEGFR in the assay system (e.g., VEGFR-expressing cell) or in vivo. Also see Example 1 for exemplary methods.

In some embodiments, fusing the VEGFR component(s) to the multivalent anti-Ang2 antibody or antigen-binding fragment thereof does not affect the biological activity of the VEGFR component(s), for example, the biological activity (e.g., blocking/neutralizing activity against VEGF) of the fused VEGFR component(s) is the same as, or within about 2-fold difference of, the biological activity of a corresponding non-fused VEGFR component (e.g., soluble VEGFR). In some embodiments, fusing the VEGFR component(s) to the multivalent anti-Ang2 antibody or antigen-binding fragment thereof reduces (e.g., reducing at least about 2-fold) the biological activity of the VEGFR component(s) compared to a corresponding non-fused VEGFR component. In some embodiments, fusing the VEGFR component(s) to the multivalent anti-Ang2 antibody or antigen-binding fragment thereof increases (e.g., increasing at least about 2-fold) the biological activity (e.g., enhances the blocking/neutralizing activity against VEGF) of the VEGFR component(s) compared to a corresponding non-fused VEGFR component.

### Linkers

In some embodiments, the anti-Ang2/VEGF-trap antibody fusion protein may comprise one or more linkers between the VEGFR component and the multivalent (e.g., bivalent) anti-Ang2 antibody (e.g., full length antibody) or antigen-binding fragment thereof, between two or more Ig-like domains of the VEGFR component, between two or more VEGFR components, between two or more anti-Ang2 binding domains of the multivalent anti-Ang2 antibody or antigen-binding fragment thereof, or between the anti-Ang2 binding domain and a connecting moiety (e.g., Fc domain, or albumin protein) of the multivalent anti-Ang2 antibody or antigen-binding fragment thereof that connects two or more anti-Ang2 binding domains. The length, the degree of flexibility and/or other properties of the linker(s) used in the antibody fusion protein may have some influence on properties, including but not limited to the affinity, specificity or avidity for Ang2, as well as for the VEGFR component, and/or their corresponding biological activities (e.g., binding to Ang2 and activating Tie2, blocking/neutralizing VEGF). For example, longer linkers may be selected to ensure that two adjacent domains do not sterically interfere with one another. In some embodiment, a linker (such as peptide linker) comprises flexible residues (such as glycine and serine) so that the adjacent domains are free to move relative to each other. For example, a glycine-serine doublet can be a suitable peptide linker. In some embodiments, the linker is a non-peptide linker. In some embodiments, the linker is a peptide linker. In some embodiments, the linker is a non-cleavable linker. Other linker considerations include the effect on physical or pharmacokinetic properties of the resulting antibody fusion protein, such as solubility, lipophilicity, hydrophilicity, hydrophobicity, stability (more or less stable as well as planned degradation), rigidity, flexibility, immunogenicity, modulation of antibody/VEGFR binding, the ability to be incorporated into a micelle or liposome, and the like.

In some embodiments, the antibody fusion protein comprises two or more linkers. In some embodiments, the two or more linkers are the same. In some embodiments, the two or more linkers are different (e.g., different from each other). In some embodiments, the one or more linkers are flexible linkers. In some embodiments, the one or more linkers are stable linkers. In some embodiments, some of the linkers are flexible, while others are stable. In general, a linker does not affect or significantly affect the proper fold and conformation formed by the configuration of the antibody fusion protein. In some embodiments, the linker confers flexibility and spatial space for each portion of the antibody fusion protein, such as allowing Ang2-anti-Ang2 binding domain binding, allowing VEGF-VEGFR component binding, providing flexibility and/or sufficient space between two binding domains or domain subunits (e.g., Ig-like domains) to ensure proper binding domain activity (binding affinity and/or bioactivity), etc.

### Peptide Linkers

Peptide linkers can be of any length. In some embodiments, the peptide linker is from about 1 amino acid (aa) to about 10 aa long, from about 2 aa to about 15 aa long, from about 3 aa to about 12 aa long, from about 1 amino acid to about 20 aa long, from about 20 aa to about 30 aa long, from about 1 amino acid to about 30 aa long, from about 10 aa to about 30 aa long, from about 1 amino acid to about 50 aa long, from about 2 aa to about 18 aa long, from about 2 aa to about 10 aa long, from about 5 aa to about 20 aa long, from about 10 aa to about 20 aa long, or from about 6 aa to about 30 aa long. In some embodiments, the peptide linker is about any of 1, 2, 3, 4, 5, 6, 7, 8, 9, 10, 11, 12, 13, 14, 15, 16, 17, 18, 19 or 20 amino acids long. In some embodiments, the peptide linker is about any of 21, 22, 23, 24, 25, 26, 27, 28, 29, or 30 amino acids long. In some embodiments, the peptide linker is about any of 31, 32, 33, 34, 35, 36, 37, 38, 39, 40, 41, 42, 43, 44, 45, 46, 47, 48, 49, or 50 amino acids long. In some embodiments, the linker is about 12 to about 15 amino acids in length.

In some embodiments, the linker is a stable linker (e.g., not cleavable by protease, especially MMPs).

A peptide linker can have a naturally occurring sequence or a non-naturally occurring sequence. For example, a sequence derived from the hinge region of a heavy chain only antibody can be used as a linker. See, for example, WO1996/34103. In some embodiments, the peptide linker is a human IgG1, IgG2, IgG3, or IgG4 hinge or portion thereof. In some embodiments, the peptide linker is a mutated human IgG1, IgG2, IgG3, or IgG4 hinge or portion thereof. In some embodiments, the linker is a flexible linker. Exemplary flexible linkers include, but are not limited to glycine polymers (G)ₙ (SEQ ID NO: 103), glycine-serine polymers (including, for example, (GS)ₙ (SEQ ID NO: 104), (GSGGS)ₙ (SEQ ID NO: 105), (GGGGS)ₙ (SEQ ID NO: 106), and (GGGS)ₙ (SEQ ID NO: 107), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11 173-142 (1992)). The ordinarily skilled artisan will recognize that design of an antibody fusion protein can include linkers that are all or partially flexible, such that the linker can include a flexible linker portion as well as one or more portions that confer less flexible structure to provide a desired antibody fusion protein structure. In some embodiments, the linker comprises the amino acid sequence of SEQ ID NO: 30 or 31.

### Non-Peptide Linkers

Any one or all of the linkers described herein can be accomplished by any chemical reaction that will connect two molecules so long as the multivalent anti-Ang2 antibody (e.g., full length antibody) or antigen-binding fragment thereof and the VEGFR component retain their respective activities (binding activity and/or biological activity and/or Fc effector activity). This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents are useful in coupling protein molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (see Killen and Lindstrom, Jour. Immun. 133:1335-2549 (1984); Jansen et al., Immunological Reviews 62:185-216 (1982); and Vitetta et al., Science 238:1098 (1987)).

Linkers that can be applied in the present application are described in the literature (see, for example, Ramakrishnan, S. et al., Cancer Res. 44:201-208 (1984) describing use of MBS (M-maleimidobenzoyl-N-hydroxysuccinimide ester)). In some embodiments, non-peptide linkers used herein include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido] hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above can contain components that have different attributes, thus leading to antibody fusion proteins with differing physio-chemical properties. For example, sulfo-NHS esters of alkyl carboxylates are more stable than sulfo-NHS esters of aromatic carboxylates. NETS-ester containing linkers are less soluble than sulfo-NHS esters. Further, the linker SMPT contains a sterically hindered disulfide bond, and can form fusion protein with increased stability. Disulfide linkages, are in general, less stable than other linkages because the disulfide linkage is cleaved in vitro, resulting in less fusion protein available. Sulfo-NHS, in particular, can enhance the stability of carbodiimide couplings. Carbodiimide couplings (such as EDC) when used in conjunction with sulfo-NHS, forms esters that are more resistant to hydrolysis than the carbodiimide coupling reaction alone.

### III. Pharmaceutical Compositions

Further provided are compositions (e.g., pharmaceutical compositions) comprising any one of the anti-Ang2/VEGF-trap antibody fusion proteins described herein, and optionally a pharmaceutically acceptable carrier. Pharmaceutical compositions can be prepared by mixing an antibody fusion protein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

A reconstituted formulation can be prepared by dissolving a lyophilized antibody fusion protein in a diluent such that the protein is dispersed throughout. Exemplary pharmaceutically acceptable (safe and non-toxic for administration to a human) diluents suitable for use in the present application include, but are not limited to, sterile water, bacteriostatic water for injection (BWFI), a pH buffered solution (e.g., phosphate-buffered saline), sterile saline solution, Ringer's solution or dextrose solution, or aqueous solutions of salts and/or buffers.

In some embodiments, the pharmaceutical composition comprises a homogeneous population of anti-Ang2/VEGF-trap antibody fusion proteins described herein. A homogeneous population means the antibody fusion proteins are exactly the same to each other, e.g., same antibody fusion protein configuration, same VEGFR component, same multivalent anti-Ang2 antibody or antigen-binding fragment thereof, and same linker if any. In some embodiments, at least about 70% (such as at least about any of 75%, 80%, 85%, 90%, 95%, 96%, 97%, 98%, or 99%) of the antibody fusion proteins in the pharmaceutical composition are homogeneous.

The pharmaceutical composition is preferably to be stable, in which the antibody fusion protein here essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month, and/or stable at 2-8° C. for at least 2 years. Where the formulation is to be stored at 30° C., generally the formulation should be stable for at least 2 years at 30° C., and/or stable at 40° C. for at least 6 months. For example, the extent of aggregation during storage can be used as an indicator of protein stability. In some embodiments, the stable formulation of antibody fusion proteins described herein may comprise less than about 10% (preferably less than about 5%) of the antibody fusion proteins present as an aggregate in the formulation.

Examples of physiologically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or nonionic surfactants such as TWEEN™, polyethylene glycol (PEG), and PLURONICS™ or polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use in the present application include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). The addition of a preservative may, for example, facilitate the production of a multi-use (multiple-dose) formulation. Suitable preservatives for use in the present application include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1% to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

Sustained-release preparations may be prepared. Suitable examples of sustained-release preparations include semi-permeable matrices of solid hydrophobic polymers containing the antagonist, which matrices are in the form of shaped articles, e.g., films, or microcapsules.

The pharmaceutical compositions herein may also contain more than one active compound as necessary for the particular indication being treated, preferably those with complementary activities that do not adversely affect each other. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

The antibody fusion protein disclosed herein can be formulated as immunoliposomes. Liposomes containing the antibody fusion protein are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

### Pharmaceutical Compositions for Treating Ocular Neovascular Disorder

For treating ocular neovascular disorders such as diabetic retinopathy and AMD, the administration of the pharmaceutical composition comprising the anti-Ang2/VEGF-trap antibody fusion proteins described herein will be directly administered to the eye, e.g., topically. Topical methods of administration include, but are not limited to, eye drops, subconjunctival injection, subconjunctival implant, intravitreal injection, intravitreal implant, sub-Tenon's injection, or sub-Tenon's implant. In some embodiments, the anti-Ang2/VEGF-trap antibody fusion protein described herein, or pharmaceutical composition thereof, is administered by intravitreal injection.

Compositions suitable for topical administration are known to the art (see, for example, US Patent Application 2005/0059639). In various embodiments, compositions of the invention can comprise a liquid comprising an active agent in solution, in suspension, or both. As used herein, liquid compositions include gels. Preferably the liquid composition is aqueous. Alternatively, the composition can take form of an ointment. In a preferred embodiment, the composition is an in situ gellable aqueous composition, more preferably an in situ gellable aqueous solution. Such a composition can comprise a gelling agent in a concentration effective to promote gelling upon contact with the eye or lacrimal fluid in the exterior of the eye. Aqueous compositions of the invention have ophthalmically compatible pH and osmolality. The composition can comprise an ophthalmic depot formulation comprising an active agent for subconjunctival administration. The microparticles comprising active agent can be embedded in a biocompatible pharmaceutically acceptable polymer or a lipid encapsulating agent. The depot formulations may be adapted to release all or substantially all the active material over an extended period of time. The polymer or lipid matrix, if present, may be adapted to degrade sufficiently to be transported from the site of administration after release of all or substantially all the active agent. The depot formulation can be a liquid formulation, comprising a pharmaceutical acceptable polymer and a dissolved or dispersed active agent. Upon injection, the polymer forms a depot at the injections site, e.g. by gelifying or precipitating. The composition can comprise a solid article that can be inserted in a suitable location in the eye, such as between the eye and eyelid or in the conjunctival sac, into a chamber of the eye, such as the anterior or posterior chambers or may be implanted in or on the sclera, choroidal space, or an avascularized region exterior to the vitreous, where the article releases the active agent. Solid articles suitable for implantation in the eye in such fashion generally comprise polymers and can be bioerodible or non-bioerodible. The implants may be permeable or impermeable to the active agent. In some embodiments, the implant may be positioned over an avascular region, such as on the sclera, so as to allow for transcleral diffusion of the drug to the desired site of treatment, e.g., the intraocular space and macula of the eye. Furthermore, the site of transcleral diffusion may be proximity to a site of neovascularization such as a site proximal to the macula.

A number of polymeric delivery vehicles for providing sustained release have been used in an ocular context and can be used to administer the compositions of the invention. Various polymers, e.g., biocompatible polymers, which may be biodegradable, can be used. The polymers may be homopolymers, copolymers (including block copolymers), straight, branched-chain, or cross-linked. Useful polymers include, but are not limited to, poly-lactic acid (PLA), poly-glycolic acid (PGA), poly-lactide-co-glycolide (PLGA), poly(phosphazine), poly (phosphate ester), polycaprolactones, polyanhydrides, ethylene vinyl acetate, polyorthoesters, polyethers, and poly (beta amino esters).

Poly (ortho esters) have been introduced into the eye and demonstrated favorable properties for sustained release ocular drug delivery (Einmahl, S., Invest. Opthalmol. Vis. Sci., 43(5), 2002). Polylactide particles have been used to target an agent to the retina and RPE following intravitreous injection of a suspension of such particles (Bourges, J-L, et al, Invest. Opthalmol. Vis. Sci., 44(8), 2003).

A method of making a sustained release formulation involves combining or mixing the antibody fusion protein described herein with a polymeric component to form a mixture. The mixture may then be extruded, compressed, molded, etc., to form a single composition. Optionally, heat and/or pressure can be used. The single composition may then be processed to form individual implants or particles suitable for placement in an eye of a patient. Additional methods for incorporating therapeutically active agents into polymeric matrices are known in the art. The polymeric matrix can be formed into various shapes such as rods, disks, wafers, etc., which may have a range of different dimensions (e.g., length, width, etc.) and volumes. Exemplary shapes include spherical, cylindrical, helical, coil-shaped or helical, screw-shaped, cubical, conical, ellipsoidal, biconvex, hemispherical or near-hemispherical etc.

### IV. Methods of Treating Diseases

Vascular endothelial proliferation and angiogenesis are important components of a variety of diseases and disorders including tumor growth and metastasis, rheumatoid arthritis, psoriasis, atherosclerosis, hemangiomas, diabetic retinopathy, retrolental fibroplasia, neovascular glaucoma, macular degeneration and related conditions (e.g., AMD), hemangiomas, immune rejection of transplanted corneal tissue and other tissues, infection, and chronic inflammation. The anti-Ang2/VEGF-trap antibody fusion proteins described herein are useful for a variety of applications, such as in diagnosis, molecular assays, and therapy. In some embodiments, there is provided a method of treating a disease or a condition (e.g., cancer, or non-neoplastic disorder) in an individual (e.g., human), comprising administering to the individual an effective amount of (1) an antibody fusion protein comprising: i) a multivalent (e.g., bivalent) anti-Ang2 antibody (e.g., full length antibody) or antigen-binding fragment thereof, and ii) a VEGFR component, wherein the multivalent anti-Ang2 antibody or antigen-binding fragment thereof does not inhibit the binding between Ang2 and Tie2; and (2) optionally a pharmaceutical acceptable carrier. In some embodiments, there is provided a method of treating a disease or a condition (e.g., cancer, or non-neoplastic disorder) in an individual (e.g., human), comprising administering to the individual an effective amount of any of the anti-Ang2/VEGF-trap antibody fusion proteins described herein, or a pharmaceutical composition thereof. The route of administration is in accordance with known and accepted methods, such as by single or multiple bolus or infusion over a long period of time in a suitable manner, e.g., injection or infusion by subcutaneous, intravenous, intraperitoneal, intramuscular, intra-arterial, intralesional or intraarticular routes, topical administration, inhalation or by sustained release or extended-release means. In some embodiments, the pharmaceutical composition is administered locally, such as intratumorally, or intravitreally.

### Non-Neoplastic Disorders

In some embodiments, the non-neoplastic disorder that can be treated by the present invention is not associated with VEGF expression. In some embodiments, the non-neoplastic disorder that can be treated by the present invention is associated with VEGF expression (such as VEGF overexpression). Overexpression of VEGF can cause non-neoplastic conditions such as rheumatoid arthritis, psoriasis, atherosclerosis, hemangiomas, immune rejection of transplanted corneal tissue and other tissues, chronic inflammation, and ocular neovascular disorders (e.g. diabetic retinopathy, retrolental fibroplasia, neovascular glaucoma, and macular degeneration and related conditions such as AMD). In some embodiments, the non-neoplastic disorder that can be treated by the present invention is associated with aberrant Ang2 expression, such as Ang2 overexpression, or insufficient Ang2 expression (in which case the antibody fusion protein described herein may help cluster Ang2 to facilitate Tie2 binding). In some embodiments, the non-neoplastic disorder that can be treated by the present invention is associated with aberrant Tie2 activity, such as insufficient Tie2 activation (in which case the antibody fusion protein described herein may help cluster Ang2 to facilitate Tie2 binding, Tie2 receptor clustering and activation).

In some embodiments, there is provided a method of treating a non-neoplastic disorder (such as ocular neovascular disorder, e.g. diabetic retinopathy and AMD) in an individual (e.g., human), comprising administering to the individual an effective amount of any of the anti-Ang2/VEGF-trap antibody fusion proteins described herein, or a pharmaceutical composition thereof. In some embodiments, the non-neoplastic disorder is associated with VEGF overexpression. In some embodiments, the non-neoplastic disorder is associated with Ang2 overexpression. In some embodiments, the non-neoplastic disorder is associated with reduced or insufficient Tie2 activation. In some embodiments, the non-neoplastic disorder is selected from the group consisting of rheumatoid arthritis, psoriasis, atherosclerosis, hemangiomas, transplant rejection (e.g., rejection of transplanted corneal tissue or other tissue), chronic inflammation, infection, and an ocular neovascular disorder (e.g. diabetic retinopathy, retrolental fibroplasia, neovascular glaucoma, and macular degeneration and related conditions (e.g., AMD)). In some embodiments, the antibody fusion protein described herein or pharmaceutical composition thereof is administered systemically (e.g., intravenously).

Rheumatoid arthritis is a chronic disease which can exhibit a variety of systemic manifestations. This disease has an unknown etiology and characteristically exhibits a persistent inflammatory synovitis which usually involves peripheral joints in a symmetric distribution. Complement-mediated inflammation which causes cartilage destruction, bone erosions and, ultimately, joint deformities is the most important feature of this disease. Methods provided herein are thus useful for treatment of rheumatoid arthritis.

Psoriasis (psoriasis vulgaris) is a chronic inflammatory skin disease characterized by red, scaly, raised plaques. The disease process is driven by T-cell infiltration and associated elevation in cytokine levels leading to increased cell division and aberrant differentiation, resulting in the psoriatic phenotype. Plaque psoriasis has a worldwide prevalence of 2-3%, and is a chronic, recurrent skin condition with varying degrees of severity. Psoriasis can profoundly impact a patient's quality of life, causing disability of physical and mental functioning comparable to other major medical diseases such as type 2 diabetes, hypertension, myocardial infarction, depression, and arthritis, it is also associated with serious co-morbidities, including psoriatic arthritis, depression, malignancy, metabolic syndrome, cardiovascular morbidity and mortality and autoimmune diseases, such as inflammatory bowel disease (iBD).

Atherosclerosis is a disease of the arterial wall in which the layer thickens, causing narrowing of the channel and thus, impairing blood flow. Atherosclerosis may occur in any area of the body, but can be most damaging to a subject when it occurs in the heart, brain or blood vessels leading to the brain stem. Atherosclerosis includes thickening and hardening of artery walls or the accumulation of fat, cholesterol and other substances that form atheromas or plaques. Atherosclerosis may result also from calcification, hemorrhage, ulceration, thrombosis, and/or trauma.

Hemangiomas are noncancerous growths that form due to an abnormal collection of blood vessels. They are often found on the skin or internal organs, particularly the liver, and are usually congenital.

In some embodiments, the disease to be treated by the present invention is chronic inflammation, including but are not limited to, asthma, chronic peptic ulcer, tuberculosis, rheumatoid arthritis, chronic periodontitis, ulcerative colitis and Crohn's disease, chronic sinusitis, and chronic active hepatitis. Chronic inflammation can result from failure to eliminate whatever was causing an acute inflammation, an autoimmune response to a self-antigen, or a chronic irritant of low intensity that persists.

### Ocular Neovascular Disorder

In some embodiments, the non-neoplastic disorder is an ocular neovascular disorder. Thus in some embodiments, there is provided a method of treating an ocular neovascular disorder (e.g., diabetic retinopathy or AMD such as wet AMD) in an individual (e.g., human), comprising administering to the individual an effective amount of any of the anti-Ang2/VEGF-trap antibody fusion proteins described herein, or a pharmaceutical composition thereof. In some embodiments, the ocular neovascular disorder is associated with one or more of choroidal neovascularization, vascular leak, and retinal edema. In some embodiments, the administration of the antibody fusion protein described herein or pharmaceutical composition thereof is selected from the group consisting of eye drops, subconjunctival injection, subconjunctival implant, intravitreal injection, intravitreal implant, sub-Tenon's injection, and sub-Tenon's implant. In some embodiments, the antibody fusion protein described herein or pharmaceutical composition thereof is administered by intravitreal injection. In some embodiments, the method further comprises subjecting the individual to an additional ocular therapy. In some embodiments, the method of treating an ocular neovascular disorder has one or more of the following biological activities: (1) inhibiting or preventing drusen formation; (2) causing a reduction in drusen number and/or size (drusen regression); (3) causing a reduction in or prevent lipofuscin deposits; (4) inhibiting or preventing visual loss or slow the rate of visual loss; (5) inhibiting choroidal neovascularization or slowing the rate of choroidal neovascularization; (6) causing a reduction in size and/or number of lesions characterized by choroidal neovascularization; (7) inhibiting choroidal neovascularization or slow the rate of retinal neovascularization; (8) causing a reduction in size and/or number of lesions characterized by retinal neovascularization; (9) improving visual acuity and/or contrast sensitivity; (10) reducing macular edema and/or reducing abnormal macular thickness; (11) inhibiting or preventing photoreceptor or RPE cell atrophy or apoptosis, or reducing the rate of photoreceptor or RPE cell atrophy or apoptosis; (12) inhibiting or preventing progression of non-exudative macular degeneration to exudative macular degeneration; and (13) preventing the development of or treating ocular fibrosis, e.g., by converting unclustered Ang2 to clustered form in the presence of the antibody fusion protein, followed by activation of Tie2 signaling.

Several ocular disorders involve alterations in angiogenesis. Non-limiting examples of ocular neovascular disorders that may be treated according to the methods of the invention include macular degeneration and related conditions (e.g., exudative AMD such as exudative wet AMD), diabetic retinopathy, angiod streaks, pathological myopia, ocular histoplasmosis syndrome, breaks in Bruch's membrane, macular edema (including diabetic macular edema), sarcoidosis and uveitis. Additional examples of disorders that may be treated by the disclosed methods include atrophic AMD, keratoconus, Sjogren's syndrome, myopia, ocular tumors, corneal graft rejection, corneal injury, neovascular glaucoma, corneal ulceration, corneal scarring, proliferative vitreoretinopathy, retinopathy of prematurity, retinal degeneration, chronic glaucoma, retinal detachment, and sickle cell retinopathy.

Diabetic retinopathy, the third leading cause of adult blindness (accounting for almost 7% of blindness in the USA), is associated with extensive angiogenic events. Nonproliferative retinopathy is accompanied by the selective loss of pericytes within the retina, and their loss results in dilation of associated capillaries dilation and a resulting increase in blood flow. In the dilated capillaries, endothelial cells proliferate and form outpouchings, which become microaneurysms, and the adjacent capillaries become blocked so that the area of retina surrounding these microaneurysms is not perfused. Eventually, shunt vessels appear between adjacent areas of micro aneurysms, and the clinical picture of early diabetic retinopathy with micro aneurysms and areas of nonperfused retina is seen. The microaneurysms leak and capillary vessels may bleed, causing exudates and hemorrhages. Once the initial stages of background diabetic retinopathy are established, the condition progresses over a period of years, developing into proliferative diabetic retinopathy and blindness in about 5% of cases. Proliferative diabetic retinopathy occurs when some areas of the retina continue losing their capillary vessels and become nonperfused, leading to the appearance of new vessels on the disk and elsewhere on the retina. These new blood vessels grow into the vitreous and bleed easily, leading to preretinal hemorrhages. In advanced proliferative diabetic retinopathy, a massive vitreous hemorrhage may fill a major portion of the vitreous cavity. In addition, the new vessels are accompanied by fibrous tissue proliferation that can lead to traction retinal detachment.

Diabetic retinopathy is associated primarily with the duration of diabetes mellitus; therefore, as the population ages and diabetic patients live longer, the prevalence of diabetic retinopathy will increase. Laser therapy is currently used in both nonproliferative and proliferative diabetic retinopathy. Focal laser treatment of the leaking microaneurysms surrounding the macular area reduces visual loss in 50% of patients with clinically significant macular edema. In proliferative diabetic retinopathy, panretinal photocoagulation results in several thousand tiny burns scattered throughout the retina (sparing the macular area); this treatment reduces the rate of blindness by 60 percent. Early treatment of macular edema and proliferative diabetic retinopathy prevents blindness for 5 years in 95% of patients, whereas late treatment prevents blindness in only 50 percent. Therefore, early diagnosis and treatment are essential.

AMD affects approximately one in ten Americans over the age of 65. AMD is characterized by a series of pathologic changes in the macula, the central region of the retina, which is accompanied by decreased visual acuity, particularly affecting central vision. AMD involves the single layer of cells called the retinal pigment epithelium that lies immediately beneath the sensory retina. These cells nourish and support the portion of the retina in contact with them, i.e., the photoreceptor cells that contain the visual pigments. The retinal pigment epithelium lies on the Bruch membrane, a basement membrane complex which, in AMD, thickens and becomes sclerotic. New blood vessels may break through the Bruch membrane from the underlying choroid, which contains a rich vascular bed. These vessels may in turn leak fluid or bleed beneath the retinal pigment epithelium and also between the retinal pigment epithelium and the sensory retina. Subsequent fibrous scarring disrupts the nourishment of the photoreceptor cells and leads to their death, resulting in a loss of central visual acuity. This type of age-related maculopathy is called the “wet” type because of the leaking vessels and the subretinal edema or blood. The wet type accounts for only 10% of age-related maculopathy cases but results in 90% of cases of legal blindness from macular degeneration in the elderly. The “dry” type of age-related maculopathy involves disintegration of the retinal pigment epithelium along with loss of the overlying photoreceptor cells. The dry type reduces vision but usually only to levels of 20/50 to 20/100.

AMD is accompanied by distortion of central vision with objects appearing larger or smaller or straight lines appearing distorted, bent, or without a central segment. In the wet type of AMD, a small detachment of the sensory retina may be noted in the macular area, but the definitive diagnosis of a subretinal neovascular membrane requires fluorescein angiography. In the dry type, drusen may disturb the pigmentation pattern in the macular area. Drusen are excrescences of the basement membrane of the retinal pigment epithelium that protrude into the cells, causing them to bulge anteriorly; their role as a risk factor in age-related maculopathy is unclear. Laser treatment is used in the wet type of age-related maculopathy and initially obliterates the neovascular membrane and prevents further visual loss in about 50% of patients at 18 months. By 60 months, however, only 20% still have a substantial benefit.

Macular edema is associated with a variety of eye disorders including AMD, diabetic retinopathy, inflammatory conditions such as anterior or posterior uveitis, etc. The macula becomes thickened as a result of the accumulation of fluid that leaks from weakened or otherwise abnormal blood vessels into nearby tissues. Leakage of blood or other fluids and the resulting increase in macular thickness can lead to acute alterations in visual acuity, color perception, etc. Thus macular edema can contribute to the visual disturbances and loss experienced by individuals suffering from AMD and a variety of other eye disorders.

In some embodiments, there is provided a method of treating or preventing one or more aspects or symptoms of AMD or diabetic retinopathy in an individual (e.g., human), including, but not limited to, neovascularization (such as choroidal neovascularization or CNV), vascular leak, and/or retinal edema, formation of ocular drusen, inflammation in the eye or eye tissue, loss of photoreceptor cells, loss of vision (including for example visual acuity and visual field), and retinal detachment, by administering to the individual an effective amount of the anti-Ang2/VEGF-trap antibody fusion protein described herein or pharmaceutical composition thereof. Treatments of other aspects of AMD are also contemplated, such as photoreceptor degeneration, RPE degeneration, retinal degeneration, chorioretinal degeneration, cone degeneration, retinal dysfunction, retinal damage in response to light exposure (such as constant light exposure), damage of the Bruch's membrane, loss of RPE function, loss of integrity of the histoarchitecture of the cells and/or extracellular matrix of the normal macular, loss of function of the cells in the macula, photoreceptor dystrophy, mucopolysaccharidoses, rod-cone dystrophies, cone-rod dystrophies, anterior and posterior uvitis, and diabetic neuropathy.

Suppression of a neovascular disorder can be evaluated by any accepted method of measuring whether angiogenesis is slowed or diminished. This includes direct observation and indirect evaluation such as by evaluating subjective symptoms or objective physiological indicators. Treatment efficacy, for example, may be evaluated based on the prevention or reversal of neovascularization, microangiopathy, endothelial glycocalyx shedding, vascular leakage or vascular edema or any combination thereof. Treatment efficacy for evaluating suppression of an ocular neovascular disorder may also be defined in terms of stabilizing or improving visual acuity. For example, improvement of clinical symptoms are monitored by, e.g., indirect ophthalmoscopy, fundus photography, fluorescein angiopathy, electroretinography, external eye examination, slit lamp biomicroscopy, applanation tonometry, pachymetry, and autorefaction.

### Cancer

In some embodiments, there is provided a method of treating a cancer (such as solid tumor, or cancer with aberrant VEGF expression, activity and/or signaling) in an individual (e.g., human), comprising administering to the individual an effective amount of any of the anti-Ang2/VEGF-trap antibody fusion proteins described herein, or a pharmaceutical composition thereof. In some embodiments, the cancer is a solid tumor (such as lung cancer, liver cancer, skin cancer (e.g., melanoma), brain cancer, breast cancer, ovarian cancer, cervical cancer, prostate cancer, colorectal cancer, renal cancer, or bladder cancer). In some embodiments, the antibody fusion protein or pharmaceutical composition thereof is administered systemically (e.g., intravenously). In some embodiments, the antibody fusion protein or pharmaceutical composition thereof is administered locally (e.g., intratumorally). In some embodiments, the method further comprises subjecting the individual to an additional cancer therapy (such as surgery, radiation, chemotherapy, immunotherapy, hormone therapy, or a combination thereof). In some embodiments, the method of treating cancer has one or more of the following biological activities: (1) killing cancer cells; (2) inhibiting proliferation of cancer cells; (3) inducing immune response in a tumor (e.g., inducing infiltration of immune effector cells to tumor site, inducing immune cell proliferation, differentiation and/or activation, and/or inducing pro-inflammatory cytokine secretion by immune cells); (4) reducing tumor size; (5) alleviating one or more symptoms in an individual having cancer; (6) inhibiting tumor metastasis; (7) prolonging survival; (8) prolonging time to cancer progression; (9) preventing, inhibiting, or reducing the likelihood of the recurrence of a cancer; and (10) reducing or inhibiting tumor angiogenesis. In some embodiments, the method of killing cancer cells mediated by the anti-Ang2/VEGF-trap antibody fusion protein or pharmaceutical composition described herein can achieve a tumor cell death rate of at least about any of 10%, 20%, 30%, 40%, 50%, 60%, 70%, 80%, 90%, 95%, or more. In some embodiments, the method of reducing tumor size mediated by the antibody fusion protein or pharmaceutical composition described herein can reduce at least about 10% (including for example at least about any of 20%, 30%, 40%, 60%, 70%, 80%, 90%, or 100%) of the tumor size. In some embodiments, the method of inhibiting tumor metastasis mediated by the antibody fusion protein or pharmaceutical composition described herein can inhibit at least about 10% (including for example at least about any of 20%, 30%, 40%, 60%, 70%, 80%, 90%, or 100%) of the metastasis. In some embodiments, the method of prolonging survival of an individual (e.g., human) mediated by the antibody fusion protein or pharmaceutical composition described herein can prolongs the survival of the individual by at least any of 1, 2, 3, 4, 5, 6, 7, 8, 9, 10, 11, 12, 18, or 24 months. In some embodiments, the method of prolonging time to cancer progression mediated by the antibody fusion protein or pharmaceutical composition described herein can prolong the time to cancer progression by at least any of 1, 2, 3, 4, 5, 6, 7, 8, 9, 10, 11, or 12 weeks. In some embodiments, the method of inducing immune response to a tumor can increase, enhance, or stimulate an immune response or function in a subject. In some embodiments, the immune response or function is increased, enhanced, and/or stimulated by activating effector cells (e.g., T cells, e.g., CD8+ and/or CD4+ T cells), expanding (increasing) an effector cell population, and/or killing target cells (e.g., target tumor cells) in the subject. In some embodiments, the CD4 and/or CD8 T cells in the individual have increased or enhanced priming, activation, proliferation, cytokine release and/or cytolytic activity relative to prior to the administration of the antibody fusion protein or pharmaceutical composition described herein.

The methods described herein are suitable for treating a variety of cancers, including both solid cancer and liquid cancer. The methods are applicable to cancers of all stages, including early stage cancer, non-metastatic cancer, primary cancer, advanced cancer, locally advanced cancer, metastatic cancer, or cancer in remission. The methods described herein may be used as a first therapy, second therapy, third therapy, or combination therapy with other types of cancer therapies known in the art, such as chemotherapy, surgery, hormone therapy, radiation, gene therapy, immunotherapy (such as T-cell therapy), bone marrow transplantation, stem cell transplantation, targeted therapy, cryotherapy, ultrasound therapy, photodynamic therapy, radio-frequency ablation or the like, in an adjuvant setting (i.e., the method may be carried out after the primary/definitive therapy) or a neoadjuvant setting (i.e., the method may be carried out before the primary/definitive therapy). In some embodiments, the method is used to treat an individual who has previously been treated. In some embodiments, the cancer has been refractory to prior therapy. In some embodiments, the method is used to treat an individual who has not previously been treated. In some embodiments, the cancer is partially resistant to immune checkpoint inhibitor monotherapy (e.g., partially resistant to anti-PD-1 or anti-PD-L1 antibody monotherapy treatment).

In some embodiments, the methods described herein are suitable for treating a solid cancer selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), cutaneous T-cell lymphoma (CTCL), cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

In some embodiments, the methods described herein are suitable for treating a hematologic cancer chosen from one or more of acute myeloid leukemia (AML), chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CIVIL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, the method is suitable for treating cancers with aberrant VEGF expression, activity and/or signaling include, by way of non-limiting example, carcinomas of the breast, lung, esophagus, gastric anatomy, colon, rectum, liver, ovary, cervix, endometrium, thecomas, arrhenoblastomas, endometrial hyperplasia, endometriosis, fibrosarcomas, choriocarcinoma, head and neck cancer, nasopharyngeal carcinoma, laryngeal carcinoma, hepatoblastoma, Karposi's sarcoma, melanoma, skin carcinomas, hemangioma, cavernous hemangioma, hemangioblastoma, pancreas carcinoma, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as associated with brain tumors), and Meigs' syndrome. In some embodiments, the method is suitable for treating lung cancer, liver cancer, skin cancer (e.g., melanoma), brain cancer, breast cancer, ovarian cancer, cervical cancer, prostate cancer, colorectal cancer, renal cancer, or bladder cancer.

In some embodiments, the method is suitable for treating cancers with aberrant Ang2 expression (e.g., over-expression, under-expression, lack of expression, etc.), activity and/or signaling include, by way of non-limiting example, hepatocellular carcinoma, gastric cancer, and squamous cell carcinoma (see, e.g., C. Li et al. Cancer Gene Therapy (2016) 23;295-302).

Administration of the anti-Ang2/VEGF-trap antibody fusion proteins described herein or pharmaceutical compositions thereof may be carried out in any convenient manner, including by injection or transfusion. The route of administration is in accordance with known and accepted methods, such as by single or multiple bolus or infusion over a long period of time in a suitable manner. The antibody fusion proteins described herein or pharmaceutical compositions thereof may be administered to a patient transarterially, subcutaneously, intradermally, intratumorally, intranodally, intramedullary, intramuscularly, intravenously, or intraperitoneally. In some embodiments, the antibody fusion protein described herein or pharmaceutical composition thereof is administered systemically. In some embodiments, the antibody fusion protein described herein or pharmaceutical composition thereof is administered to an individual by infusion, such as intravenous infusion. Infusion techniques for immunotherapy are known in the art (see, e.g., Rosenberg et al., New Eng. J. of Med. 319: 1676 (1988)). In some embodiments, the antibody fusion protein described herein or pharmaceutical composition thereof is administered to an individual by intradermal or subcutaneous (i.e., beneath the skin) injection. For subcutaneous injections, the antibody fusion protein described herein or pharmaceutical composition thereof may be injected using a syringe. However, other devices for administration are available such as injection devices; injector pens; auto-injector devices, needleless devices; and subcutaneous patch delivery systems. In some embodiments, the antibody fusion protein described herein or pharmaceutical composition thereof is administered by intravenous injection. In some embodiments, the antibody fusion protein described herein or pharmaceutical composition thereof is injected directly into a tumor, or a lymph node. In some embodiments, the antibody fusion protein described herein or pharmaceutical composition thereof is administered locally to a site of tumor, such as directly into tumor cells, or to a tissue having tumor cells. In some embodiments, the antibody fusion protein described herein or pharmaceutical composition thereof is administered by sustained release or extended-release means.

Dosages and desired drug concentration of the antibody fusion proteins described herein or pharmaceutical compositions thereof may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46. It is within the scope of the present application that different formulations will be effective for different treatments and different disorders, and that administration intended to treat a specific organ or tissue may necessitate delivery in a manner different from that to another organ or tissue.

When in vivo administration of the antibody fusion proteins described herein or pharmaceutical compositions thereof are used, normal dosage amounts may vary from about 1 mg/kg to about 50 mg/kg of mammal body weight depending upon the route of administration and mammal type. It is within the scope of the present application that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays. In some embodiments, the antibody fusion proteins described herein or pharmaceutical compositions thereof is administered in an amount of about 1 mg/kg to about 50 mg/kg, such as any of about 10 mg/kg to about 50 mg/kg, about 10 mg/kg to about 40 mg/kg, about 10 mg/kg to about 30 mg/kg, or about 20 mg/kg.

In some embodiments, the antibody fusion protein described herein or pharmaceutical composition thereof is administered (e.g., infused) to the individual (e.g., human) over a period of time no more than about any of 24 hours, 20 hours, 15 hours, 10 hours, 8 hours, 6 hours, 3 hours, 2 hours, 1 hours, 30 minutes, or less. In some embodiments, the antibody fusion protein described herein or pharmaceutical composition thereof is administered for a single time (e.g. bolus injection). In some embodiments, the antibody fusion protein described herein or pharmaceutical composition thereof is administered for multiple times (such as any of 2, 3, 4, 5, 6, or more times). If multiple administrations, they may be performed by the same or different routes and may take place at the same site or at alternative sites. The antibody fusion protein described herein or pharmaceutical composition thereof may be administered daily to once per year. The interval between administrations can be about any one of 24 hours to a year. Intervals can also be irregular (e.g., following tumor progression). In some embodiments, there is no break in the dosing schedule. The optimal dosage and treatment regime for a particular patient can readily be determined by one skilled in the art of medicine by monitoring the patient for signs of disease and adjusting the treatment accordingly. In some embodiments, the antibody fusion protein described herein or pharmaceutical composition thereof is administered once per day (daily), once per 2 days, once per 3 days, once per 4 days, once per 5 days, once per 6 days, once per week, once per 10 days, once every 2 weeks, once every 3 weeks, once every 4 weeks, once per month, once per 2 months, once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year.

In some embodiments, the antibody fusion protein described herein or pharmaceutical composition thereof is administered in split doses, such as about any one of 2, 3, 4, 5, or more doses. In some embodiments, the split doses are administered over about a week, a month, 2 months, 3 months, or longer. In some embodiments, the dose is equally split. In some embodiments, the split doses are about 20%, about 30% and about 50% of the total dose. In some embodiments, the interval between consecutive split doses is about 1 day, 2 days, 3 days, 1 week, 2 weeks, 3 weeks, a month, or longer. For repeated administrations over several days or longer, depending on the condition, the treatment is sustained until a desired suppression of disease symptoms occurs. However, other dosage regimens may be useful. The progress of this therapy is easily monitored by conventional techniques and assays.

### V. Vectors Encoding Antibody Fusion Proteins

The present invention also provides isolated nucleic acids encoding any of the anti-Ang2/VEGF-trap antibody fusion proteins described herein, and vectors comprising such nucleic acids. Also provided are isolated host cells (e.g., CHO cells, HEK 293 cells, Hela cells, COS cells) comprising nucleic acids encoding any of the anti-Ang2/VEGF-trap antibody fusion proteins described herein, or vectors comprising nucleic acids encoding any of the anti-Ang2/VEGF-trap antibody fusion proteins described herein. In some embodiments, the vector comprising a nucleic acid encoding any of the antibody fusion proteins described herein is suitable for replication and integration in eukaryotic cells, such as mammalian cells (e.g., CHO cells).

In some embodiments, the vector is a viral vector. Examples of viral vectors include, but are not limited to, adenoviral vectors, adeno-associated virus vectors, lentiviral vector, retroviral vectors, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals. A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. In some embodiments, adenovirus vectors are used. A number of retroviral systems and adenovirus vectors are known in the art. In some embodiments, lentivirus vectors are used. In some embodiments, self-inactivating lentiviral vectors are used. For example, self-inactivating lentiviral vectors carrying the antibody fusion protein encoding sequence(s) can be packaged with protocols known in the art. The resulting lentiviral vectors can be used to transduce a mammalian cell using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector is a non-viral vector. In some embodiments, the vector is a pTT5 vector. In some embodiments, the vector is a transposon, such as a Sleeping Beauty (SB) transposon system, or a PiggyBac transposon system. In some embodiments, the vector is a polymer-based non-viral vector, including for example, poly (lactic-co-glycolic acid) (PLGA) and poly lactic acid (PLA), poly (ethylene imine) (PEI), and dendrimers. In some embodiments, the vector is a cationic-lipid based non-viral vector, such as cationic liposome, lipid nanoemulsion, and solid lipid nanoparticle (SLN). In some embodiments, the vector is a peptide-based gene non-viral vector, such as poly-L-lysine. Any of the known non-viral vectors suitable for genome editing can be used for introducing the antibody fusion protein-encoding nucleic acid(s) to the host cells. See, for example, Yin H. et al. Nature Rev. Genetics (2014) 15:521-555; Aronovich E L et al. Hum. Mol. Genet. (2011) R1: R14-20; and Zhao S. et al. Transl. Lung Cancer Res. (2016) 5(1): 120-125, which are incorporated herein by reference. In some embodiments, any one or more of the nucleic acids or vectors encoding the antibody fusion proteins described herein is introduced to the host cells by a physical method, including, but not limited to electroporation, sonoporation, photoporation, magnetofection, hydroporation.

In some embodiments, the vector contains a selectable marker gene or a reporter gene to select cells expressing the antibody fusion proteins described herein from the population of host cells transfected through vectors. Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

In some embodiments, the vector (e.g., viral vector) comprises any one of the nucleic acids encoding the anti-Ang2/VEGF-trap antibody fusion proteins described herein. The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present invention. Promoters may be roughly categorized as constitutive promoters or regulated promoters, such as inducible promoters.

In some embodiments, the nucleic acid encoding the antibody fusion proteins described herein is operably linked to a constitutive promoter. Constitutive promoters allow heterologous genes (also referred to as transgenes) to be expressed constitutively in the host cells. Exemplary promoters contemplated herein include, but are not limited to, cytomegalovirus immediate-early promoter (CMV), human elongation factors-1alpha (hEF1α), ubiquitin C promoter (UbiC), phosphoglycerokinase promoter (PGK), simian virus 40 early promoter (SV40), chicken β-Actin promoter coupled with CMV early enhancer (CAGG), a Rous Sarcoma Virus (RSV) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter. The efficiencies of such constitutive promoters on driving transgene expression have been widely compared in a huge number of studies.

In some embodiments, the nucleic acid encoding the anti-Ang2/VEGF-trap antibody fusion proteins described herein is operably linked to an inducible promoter. Inducible promoters belong to the category of regulated promoters. The inducible promoter can be induced by one or more conditions, such as a physical condition, microenvironment of the host cells, or the physiological state of the host cells, an inducer (i.e., an inducing agent), or a combination thereof. In some embodiments, the inducing condition does not induce the expression of endogenous genes in the host cell. In some embodiments, the inducing condition is selected from the group consisting of: inducer, irradiation (such as ionizing radiation, light), temperature (such as heat), redox state, and the activation state of the host cell. In some embodiments, the inducible promoter can be an NFAT promoter, a TETON® promoter, or an NFκB promoter. In some embodiments, the inducible promoter is a tet-inducible promoter.

In some embodiments, the vector comprises more than one nucleic acids encoding the anti-Ang2/VEGF-trap antibody fusion proteins described herein, e.g., different polypeptides of the antibody fusion protein. In some embodiments, each vector comprises 2 nucleic acids encoding 2 polypeptides of the antibody fusion proteins described herein.

In some embodiments, the two or more nucleic acids encoding the anti-Ang2/VEGF-trap antibody fusion proteins described herein are operably regulated under the same promoter in the vector. In some embodiments, the two or more nucleic acids are linked in tandem via a linking sequence (e.g., IRES) or a nucleic acid sequence encoding a self-cleaving 2A peptide, such as P2A, T2A, E2A, F2A, BmCPV 2A, BmIFV 2A. In some embodiments, the nucleic acid encoding two or more polypeptides of the antibody fusion protein comprises linking sequence(s) (e.g., IRES) or nucleic acid sequence(s) encoding self-cleaving 2A peptide(s) (such as P2A, T2A, E2A, F2A, BmCPV 2A, BmIFV 2A) between the polypeptide encoding sequences. In some embodiments, the two or more nucleic acids encoding the antibody fusion proteins described herein are operably regulated under separate promoters in the vector. In some embodiments, the promoters operably linked to each nucleic acid are different. In some embodiments, the promoters operably linked to each nucleic acid are the same. In some embodiments, the antibody fusion protein described herein is encoded by two or more vectors.

### VI. Methods of Preparation

Also provided are methods of producing any of the anti-Ang2/VEGF-trap antibody fusion proteins described herein. Thus, in some embodiments, there is provided a method of producing an antibody fusion protein, comprising: (a) culturing a host cell (e.g., CHO cell) comprising any of the nucleic acids or vectors encoding the antibody fusion proteins described herein under a condition effective to express the encoded antibody fusion protein; and (b) obtaining the expressed antibody fusion protein from said host cell. In some embodiments, the method of step (a) further comprises producing a host cell comprising the nucleic acid or vector encoding the antibody fusion protein described herein. The anti-Ang2/VEGF-trap antibody fusion protein described herein may be prepared using any methods known in the art or as described herein. Also see Example 1 for exemplary methods. In some embodiments, the antibody fusion protein is expressed with eukaryotic cells, such as mammalian cells. In some embodiments, the antibody fusion protein is expressed with prokaryotic cells.

### 1. Vector Construction

Polynucleic acid sequences encoding the antibody fusion proteins of the present application can be obtained using standard recombinant techniques. Desired polynucleic acid sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in suitable hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an optional enhancer, an origin of replication, a selection marker gene, a promoter, a ribosome binding site (RB S), a signal sequence, the heterologous nucleic acid insert, and a transcription termination sequence. The origin of replication component may not be needed for mammalian expression vectors (the SV40 origin may typically be used only because it contains the early promoter). In general, plasmid vectors containing replicon and control sequences which are derived from species compatible with the host cell are used in connection with these hosts. In some embodiments, heterologous promoters are utilized, as they generally permit greater transcription and higher yields of expressed target gene as compared to the native target polypeptide promoter. The expression vector of the present application may comprise two or more promoter-cistron pairs, encoding each of the polypeptide components. Many enhancer sequences are known from mammalian genes (globin, elastase, albumin, α-fetoprotein, and insulin), may be spliced into the vector at a position 5′ or 3′ to the polypeptide encoding sequence, but is preferably located at a site 5′ from the promoter. Typical selection genes encode proteins that (a) confer resistance to antibiotics or other toxins, e.g., ampicillin, neomycin, methotrexate, or tetracycline, (b) complement auxotrophic deficiencies, (c) supply critical nutrients not available from complex media, or (d) serve as identification marker (e.g., GFP).

Vectors and promoters suitable for expression in prokaryotic and eukaryotic cells are well known in the art. For example, promoters suitable for use with prokaryotic hosts include the PhoA promoter, the -galactamase and lactose promoter systems, a tryptophan (trp) promoter system and hybrid promoters such as the tac or the trc promoter, etc. Also see section “V. Vectors encoding antibody fusion proteins” above.

In some embodiments, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e., cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In mammalian cell expression, mammalian signal sequences as well as viral secretory leaders, for example, the herpes simplex gD signal, are available. The DNA for such precursor region is ligated in reading frame to DNA encoding the antibody fusion protein of the present application.

In some embodiments, the production of a protein can occur in the cytoplasm of the host cell, and therefore does not require the presence of secretion signal sequences within each cistron. In some embodiments, polypeptide components are expressed, folded, and assembled to form the protein within the cytoplasm. Certain prokaryotic host strains (e.g., the E. coli trxB⁻ strains) provide cytoplasm conditions that are favorable for disulfide bond formation, thereby permitting proper folding and assembly of expressed protein subunits. See Proba and Pluckthun, Gene, 159:203 (1995).

In some embodiments, the quantitative ratio of expressed polypeptide components can be modulated in order to maximize the yield of secreted and properly assembled antibody fusion protein. Such modulation is accomplished at least in part by simultaneously modulating translational strengths for the polypeptide components. One technique for modulating translational strength is disclosed in Simmons et al., U.S. Pat. No. 5,840,523.

### 2. Host Cells

Prokaryotic host cells suitable for expressing the antibody fusion proteins of the present application include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, E. coli cells are used. Suitable E. coli strains are well known in the art. Methods of selecting the appropriate bacteria host for various plasmids are when well-known in the art.

Suitable eukaryote host cells include vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Typically, the host cell should secrete minimal amounts of proteolytic enzymes, and additional protease inhibitors may desirably be incorporated in the cell culture.

### 3. Protein Production

Host cells are transformed or transfected with the above-described expression vectors and cultured in conventional nutrient media or any media modified as appropriate for inducing promoters, selecting transformants, or amplifying the genes encoding the desired sequences. The calcium treatment employing calcium chloride is generally used for bacterial cells that contain substantial cell-wall barriers. Another method for transformation employs polyethylene glycol/DMSO. Other techniques such as electroporation, liposome-mediated transfection, etc. are also suitable.

Examples of suitable media for prokaryotic cells include luria broth (LB) plus necessary nutrient supplements. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing eukaryotic host cells. In some embodiments, the media also contains a selection agent, chosen based on the construction of the expression vector, to selectively permit growth of host cells containing the expression vector. For example, ampicillin is added to media for growth of cells expressing ampicillin resistant gene. In some embodiments, media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

The expressed antibody fusion proteins can be in the cytoplasm, or secreted into and recovered from the periplasm for prokaryotic cells. Protein recovery typically involves disrupting the host cells, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. A protease inhibitor such as PMSF may be included in any of the foregoing steps to inhibit proteolysis and antibiotics may be included to prevent the growth of adventitious contaminants. To minimize proteolysis of expressed heterologous proteins (especially those that are proteolytically sensitive), certain host strains deficient for proteolytic enzymes can be used. The expressed polypeptides can be further isolated and identified using commonly known methods such as polyacrylamide gel electrophoresis (PAGE) and Western blot assay.

Alternatively, protein production is conducted in large quantity by a fermentation process with prokaryotic host cells. Various large-scale fed-batch fermentation procedures are available for production of recombinant proteins. During the fermentation process, induction of protein expression is typically initiated after the cells have been grown under suitable conditions to a desired density, e.g., an OD₅₅₀ of about 180-220, at which stage the cells are in the early stationary phase.

### 4. Protein Purification

The antibody fusion proteins produced herein are further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatography on an anion exchange resin, hydroxylapatite chromatography, chromatofocusing, SDS-PAGE, ammonium sulfate precipitation, dialysis, and gel filtration using, for example, Sephadex G-75.

In some embodiments, Protein A immobilized on a solid phase is used for immunoaffinity purification of the antibody fusion protein comprising an Fc domain. The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants. The solid phase is then washed to remove contaminants non-specifically bound to the solid phase. Finally, the antibody fusion proteins of interest are recovered from the solid phase by elution. Following any preliminary purification step(s), the mixture comprising the protein construct of interest and contaminants may be subjected to low pH hydrophobic interaction chromatography using an elution buffer at a pH between about 2.5-4.5, preferably performed at low salt concentrations (e.g., from about 0-0.25M salt).

### VII. Articles of Manufacture and Kits

Further provided are kits, unit dosages, and articles of manufacture comprising any of the anti-Ang2/VEGF-trap antibody fusion proteins described herein. In some embodiments, a kit is provided which contains any one of the pharmaceutical compositions described herein and preferably provides instructions for its use, such as for use in the treatment of the disorders described herein (e.g., cancer, or non-neoplastic disorder).

Kits of the invention include one or more containers comprising an anti-Ang2/VEGF-trap antibody fusion protein described herein for treating a disease. For example, the instructions comprise a description of administration of the antibody fusion protein to treat a disease, such as cancer. The kit may further comprise a description of selecting an individual (e.g., human) suitable for treatment based on identifying whether that individual has the disease and the stage of the disease. The instructions relating to the use of the antibody fusion protein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The kits of the present application are in suitable packaging. Suitable packaging includes, but is not limited to, vials, bottles, jars, flexible packaging (e.g., sealed Mylar or plastic bags), and the like. Also contemplated are packages for use in combination with a specific device, such as an infusion device such as a minipump. A kit may have a sterile access port (for example, the container may be an intravenous solution bag or a vial having a stopper pierceable by a hypodermic injection needle). At least one active agent in the composition is an anti-Ang2/VEGF-trap antibody fusion protein as described herein. The container may further comprise a second pharmaceutically active agent. The kits may optionally provide additional components such as buffers and interpretive information. Normally, the kit comprises a container and a label or package insert(s) on or associated with the container. In some embodiments, the kit provides instruction(s) and/or tool(s) for administration of the antibody fusion protein described herein or pharmaceutical composition thereof as eye drops, subconjunctival injection, subconjunctival implant, intravitreal injection, intravitreal implant, sub-Tenon's injection, or sub-Tenon's implant.

The present application thus also provides articles of manufacture, which include vials (such as sealed vials), bottles, jars, flexible packaging, and the like. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example, the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, syringes, or tool(s) for administration as eye drops, subconjunctival injection, subconjunctival implant, intravitreal injection, intravitreal implant, sub-Tenon's injection, or sub-Tenon's implant.

The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

The examples below are intended to be purely exemplary of the invention and should therefore not be considered to limit the invention in any way. The following examples and detailed description are offered by way of illustration and not by way of limitation.

### Example 1: Construction and Expression of Anti-Ang2/VEGF-Trap Antibody Fusion Proteins

A series of anti-Ang2/VEGF-trap bispecific antibody fusion proteins (BsFps) were designed based on anti-Ang2 monoclonal antibodies (mAbs) and VEGF-trap comprising Ig-like domain 2 of VEGFR-1-Ig-like domain 3 of VEGFR-2 (Flt1d2-Flk1d3) chimeric fusion (SEQ ID NO: 32). For each BsFp, VEGF-trap was fused to either the light chain of an anti-Ang2 mAb via an “E4” linker (SEQ ID NO: 31), or the heavy chain of an anti-Ang2 mAb via a (G₄S)₃ linker (SEQ ID NO: 30). Each BsFp construct either has two identical heavy chain fusion polypeptides and two identical antibody light chains, or two identical antibody heavy chains and two identical light chain fusion polypeptides. DNA sequence encoding each polypeptide was inserted into a pTT5 vector between EcoRI and HindIII restriction sites. Each plasmid also contained a sequence encoding a signal peptide for polypeptides to be secreted into the growth medium. CHO-3E7 cells transfected with expression plasmids were cultured at 37° C. and 100 rpm for 6 days. The supernatant fraction was collected by centrifugation and BsFp protein was purified on Protein A column. BsFp constructs are shown in Table 1.

TABLE 1
Anti-Ang2/VEGF-trap BsFp constructs

| | | | VEGF- | VEGF- |
|---|---|---|---|---|
| | | | trap HC | trap LC |
| BsFp name | HC or HC-fusion | LC or LC-fusion | fusion | fusion |
| 5A7B9-VH3.2-vegfr-VL1 | 5A7B9-VH3.2-vegfr | 5A7B9-VL1 | C′ of HC | / |
| | (SEQ ID NO: 50) | (SEQ ID NO: 42) | | |
| 5A7B9-VH3-vegfr-VL1.1 | 5A7B9-VH3-vegfr | 5A7B9-VL1.1 | C′ of HC | / |
| | (SEQ ID NO: 51) | (SEQ ID NO: 41) | | |
| 5A7B9-VH3-vegfr-VL1 | 5A7B9-VH3-vegfr | 5A7B9-VL1 | C′ of HC | / |
| | (SEQ ID NO: 51) | (SEQ ID NO: 42) | | |
| 31E2D4-VH4-vegfr-VL1 | 31E2D4-VH4-vegfr | 5A7B9-VL1 | C′ of HC | / |
| | (SEQ ID NO: 52) | (SEQ ID NO: 42) | | |
| 7F10B2-VH5-vegfr-VL1 | 7F10B2-VH5-vegfr | 7F10B2-VL1 | C′ of HC | / |
| | (SEQ ID NO: 53) | (SEQ ID NO: 45) | | |
| 7F10B2-VH3-vegfr-VL3 | 7F10B2-VH3-vegfr | 7F10B2-VL3 | C′ of HC | / |
| | (SEQ ID NO: 54) | (SEQ ID NO: 46) | | |
| 5F7D7-VH3-vegfr-VL5 | 5F7D7-VH3-vegfr | 5F7D7-VL5 | C′ of HC | / |
| | (SEQ ID NO: 55) | (SEQ ID NO: 44) | | |
| 42C3A4-VH1-vegfr-VL1 | 42C3A4-VH1-vegfr | 42C3A4-VL1 | C′ of HC | / |
| | (SEQ ID NO: 74) | (SEQ ID NO: 47) | | |
| hABTAA-VHvegfr-VL | hABTAA-VH-vegfr | hABTAA-VL | C′ of HC | / |
| | (SEQ ID NO: 78) | (SEQ ID NO: 48) | | |
| vegfr-5A7B9-VH3.2-VL1 | vegfr-5A7B9-VH3.2 | 5A7B9-VL1 | N′ of HC | / |
| | (SEQ ID NO: 56) | (SEQ ID NO: 42) | | |
| vegfr-5A7B9-VH3-VL1.1 | vegfr-5A7B9-VH3 | 5A7B9-VL1.1 | N′ of HC | / |
| | (SEQ ID NO: 57) | (SEQ ID NO: 41) | | |
| vegfr-5A7B9-VH3-VL1 | vegfr-5A7B9-VH3 | 5A7B9-VL1 | N′ of HC | / |
| | (SEQ ID NO: 57) | (SEQ ID NO: 42) | | |
| vegfr-31E2D4-VH4-VL1 | vegfr-31E2D4-VH4 | 5A7B9-VL1 | N′ of HC | / |
| | (SEQ ID NO: 58) | (SEQ ID NO: 42) | | |
| vegfr-7F10B2-VH5-VL1 | vegfr-7F10B2-VH5 | 7F10B2-VL1 | N′ of HC | / |
| | (SEQ ID NO: 59) | (SEQ ID NO: 45) | | |
| vegfr-7F10B2-VH3-VL3 | vegfr-7F10B2-VH3 | 7F10B2-VL3 | N′ of HC | / |
| | (SEQ ID NO: 60) | (SEQ ID NO: 46) | | |
| vegfr-5F7D7-VH3-VL5 | vegfr-5F7D7-VH3 | 5F7D7-VL5 | N′ of HC | / |
| | (SEQ ID NO: 61) | (SEQ ID NO: 44) | | |
| vegfr-42C3A4-VH1-VL1 | vegfr-42C3A4-VH1 | 42C3A4-VL1 | N′ of HC | / |
| | (SEQ ID NO: 75) | (SEQ ID NO: 47) | | |
| vegf-hABTAA-VH-VL | vegfr-hABTAA-VH | hABTAA-VL | N′ of HC | / |
| | (SEQ ID NO: 79) | (SEQ ID NO: 48) | | |
| 5A7B9-VL1-vegfr-VH3.2 | 5A7B9-VH3.2 | 5A7B9-VL1-vegfr | / | C′ of LC |
| | (SEQ ID NO: 33) | (SEQ ID NO: 62) | | |
| 5A7B9-VL1.1-vegfr-VH3 | 5A7B9-VH3 | 5A7B9-VL1.1-vegfr | / | C′ of LC |
| | (SEQ ID NO: 34) | (SEQ ID NO: 63) | | |
| 5A7B9-VL1-vegfr-VH3 | 5A7B9-VH3 | 5A7B9-VL1-vegfr | / | C′ of LC |
| | (SEQ ID NO: 34) | (SEQ ID NO: 62) | | |
| 5A7B9-VL2-vegfr-VH3 | 5A7B9-VH3 | 5A7B9-VL2-vegfr | / | C′ of LC |
| | (SEQ ID NO: 34) | (SEQ ID NO: 76) | | |
| 7F10B2-VL1-vegfr-VH5 | 7F10B2-VH5 | 7F10B2-VL1-vegfr | / | C′ of LC |
| | (SEQ ID NO: 37) | (SEQ ID NO: 64) | | |
| 7F10B2-VL3-vegfr-VH3 | 7F10B2-VH3 | 7F10B2-VL3-vegfr | / | C′ of LC |
| | (SEQ ID NO: 36) | (SEQ ID NO: 65) | | |
| 5F7D7-VL5-vegfr-VH3 | 5F7D7-VH3 | 5F7D7-VL5-vegfr | / | C′ of LC |
| | (SEQ ID NO: 35) | (SEQ ID NO: 66) | | |
| 42C3A4-VL1-vegfr-VH1 | 42C3A4-VH1 | 42C3A4-VL1-vegfr | / | C′ of LC |
| | (SEQ ID NO: 39) | (SEQ ID NO: 72) | | |
| 31E2D4-VL1-vegfr-VH4 | 31E2D4-VH4 | 5A7B9-VL1-vegfr | / | C′ of LC |
| | (SEQ ID NO: 38) | (SEQ ID NO: 62) | | |
| hABTAA-VH-VLvegfr | hABTAA-VH | hABTAA-VL-vegfr | / | C′ of LC |
| | (SEQ ID NO: 40) | (SEQ ID NO: 80) | | |
| vegfr-5A7B9-VL1-VH3.2 | 5A7B9-VH3.2 | vegfr-5A7B9-VL1 | / | N′ of LC |
| | (SEQ ID NO: 33) | (SEQ ID NO: 67) | | |
| vegfr-5A7B9-VL1.1-VH3 | 5A7B9-VH3 | vegfr-5A7B9-VL1.1 | / | N′ of LC |
| | (SEQ ID NO: 34) | (SEQ ID NO: 68) | | |
| vegfr-5A7B9-VL1-VH3- | 5A7B9-VH3 | vegfr-5A7B9-VL1 | / | N′ of LC |
| | (SEQ ID NO: 34) | (SEQ ID NO: 67) | | |
| vegfr-5A7B9-VL2-VH3 | 5A7B9-VH3 | vegfr-5A7B9-VL2 | / | N′ of LC |
| | (SEQ ID NO: 34) | (SEQ ID NO: 77) | | |
| vegfr-7F10B2-VL1-VH5 | 7F10B2-VH5 | vegfr-7F10B2-VL1 | / | N′ of LC |
| | (SEQ ID NO: 37) | (SEQ ID NO: 69) | | |
| vegfr-7F10B2-VL3-VH3 | 7F10B2-VH3 | vegfr-7F10B2-VL3 | / | N′ of LC |
| | (SEQ ID NO: 36) | (SEQ ID NO: 70) | | |
| vegfr-5F7D7-VL5 -VH3 | 5F7D7-VH3 | vegfr-5F7D7-VL5 | / | N′ of LC |
| | (SEQ ID NO: 35) | (SEQ ID NO: 71) | | |
| vegfr-42C3A4-VL1-VH1 | 42C3A4-VH1 | vegfr-42C3A4-VL1 | / | N′ of LC |
| | (SEQ ID NO: 39) | (SEQ ID NO: 73) | | |
| vegfr-31E2D4-VL1-VH4 | 31E2D4-VH4 | vegfr-5A7B9-VL1 | / | N′ of LC |
| | (SEQ ID NO: 38) | (SEQ ID NO: 67) | | |
| hABTAA-VH-vegfrVL | hABTAA-VH | vegfr-hABTAA-VL | / | N′ of LC |
| | (SEQ ID NO: 40) | (SEQ ID NO: 81) | | |

### In Vitro Functional Analysis by Phos-AKT Bioassay

### 1. HUVEC Culture and Preparation for Future Experiments

Human Primary Umbilical Vein Endothelial Cells (HUVEC; ATCC®) express Tie2 cell surface receptors. HUVECs were cultured in a 10 cm dish with human Endothelial Cell Medium-complete (ECM; ScienCell), 37° C. and 5% CO₂. When HUVECs reached 80%-90% confluency, 2 mL Accutase® cell dissociation reagent (Gibco®) was added to dissociate cells from the dish. HUVECs were then resuspended with ECM complete medium to a final concentration of about 1×10⁶ cells/mL. 50 μL of cell suspension was added to each well of a 96-well flat-bottom plate, then incubated in 37° C., 5% CO₂ incubator overnight. After discarding all cell culture medium from the wells, 50 μL fresh serum-free medium was added to each well, then cells were incubated for 16 hours at 37° C. in a 5% CO₂ incubator.

### 2. Functional Leads Screening by Single Dose

Each BsFp with single dose was used for the functional leads screening, and 4 μg/mL (4× stock) Ang2 protein (R&D system) and 40 μg/mL (4× stock) BsFp (or control) were prepared for functional screening. An hATBAA anti-Ang2 antibody served as positive control in activating Tie2 signaling through Ang2 binding. After adding 25 μL of 40 μg/mL BsFp (or control) to each well of the 96-well plate containing HUVECs in 50 μL serum-free medium (prepared as above in subsection 1), 25 μL of 4 μg/mL Ang2 protein (R&D system) was immediately added into each well followed by incubation at 37° C. in 5% CO₂ incubator for 10 minutes. HUVECs incubated with Ang2 in buffer, but without adding BsFp served as negative control.

### Phospho-Akt (Ser473) Bioassay

Phospho-Akt (Ser473) kit (Cisbio) was utilized to detect pan Akt phosphorylation on Ser473 as a readout of Tie2 activation. Supernatant from each well containing sample treated-HUVECs was discarded. 50 μL of supplemented lysis buffer (Cisbio) was added into each well, followed by incubation for 35 minutes at room temperature with shaking. After that, 16 μL cell lysate from each well was transferred to a small volume detection plate. After adding 4 μL of HTRF® pre-mixed antibodies (Ciobio) into each detection well and sealing the plate, the plate was incubated at room temperature for 4 hours. The phosphorylation level of Akt was measured by reading the fluorescence emission at 665 nm and 620 nm wavelengths on PHERAstar® (a HTRF® plate reader). A blank well served as negative detection control.

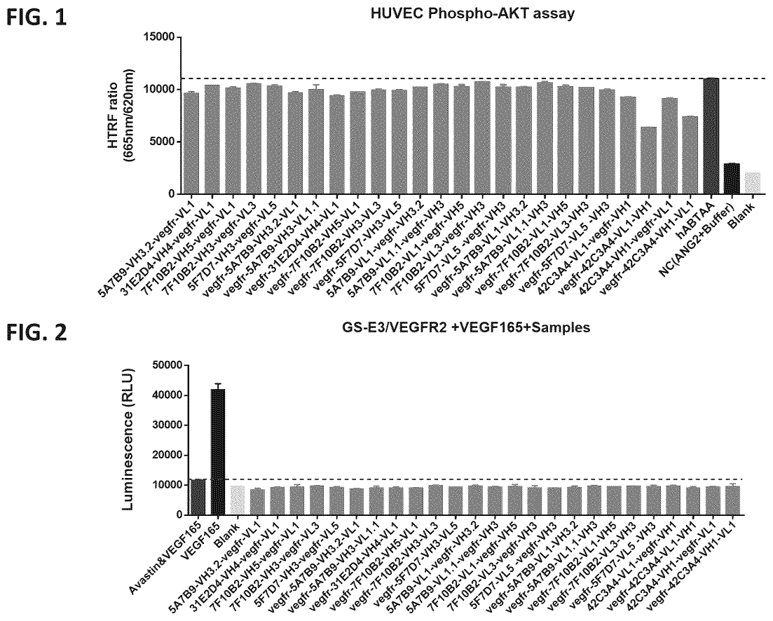

The phospho-AKT bioassay results are shown in FIG. 1. All BsFps tested significantly activated Tie2 signaling compared to the negative control (Ang2 only), indicating that VEGF-trap fusion location did not significantly affect BsFp's ability in Ang2 binding and/or Tie2 signaling activation. For 42C3A4-based BsFps, VEGF-trap fused to the N-terminus of either heavy chain (vegfr-42C3A4-VH1-VL1) or light chain (vegfr-42C3A4-VL1-VH1) slightly reduced BsFp's ability in Ang2 binding and/or Tie2 signaling activation, but still significantly higher than negative control.

### 3. Functional Leads Bioassay by Dose-Response Curves

4 μg/mL (4× stock) Ang2 protein (R&D system) and 200 μg/mL (4× stock) BsFp (or control) were prepared for dose curve assay. hATBAA anti-Ang2 antibody served as positive control. The first concentration was 200 μg/mL (4× stock) followed by 5-fold dilution for the other 7 data points. After adding 25 μL various concentrations of BsFp (or control) dilutions to each well of 96-well plate containing HUVECs in 50 μL serum-free medium (prepared as above in subsection 1), 25 μL of 4 μg/mL Ang2 protein (R&D system) was immediately added into each well followed by incubation at 37° C. in 5% CO₂ incubator for 10 minutes.

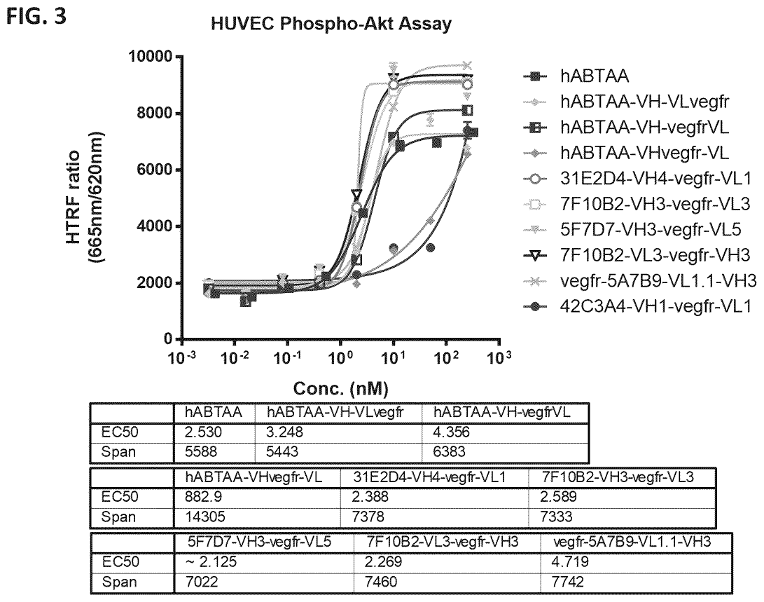

Phospho-AKT bioassay was conducted as above in subsection “Phospho-Akt (Ser473) bioassay.” As can be seen from FIG. 3, all BsFps tested effectively led to Tie2 activation except for 42C3A4-VH1-vegfr-VL1 (EC50 non-detectable) and hABTAA-VHvegfr-VL. More importantly, some BsFps, including 5F7D7-VH3-vegfr-VL5, 7F10B2-VL3-vegfr-VH3, 7F10B2-VH3-vegfr-VL3, vegfr-5A7B9-VL1.1-VH3, and 31E2D4-VH4-vegfr-VL1, showed better Tie2 activating function than hABTAA mAb with higher span value and comparable EC50 value.

### In Vitro Functional Analysis by VEGF-Blocking Bioassay

### 1. GS-E3/VEGFR2 Cell Culture and Preparation for Future Experiments

GS-E3/VEGFR2 cells expressing VEGFR2 on the cell surface and a firefly luciferase reporter gene under the control of NFAT response element were generated in-house (GenScript). GS-E3/VEGFR2 cells were cultured in a 10 cm dish with DMEM complete culture medium (Gibco) at 37° C., 5% CO₂. When GS-E3/VEGFR2 cells reached 80%-90% confluency, 2 mL Accutase® cell dissociation reagent (Gibco®) was added to dissociate cells from the dish. After centrifugation, GS-E3/VEGFR2 cells were resuspended with DMEM complete culture medium to a final concentration of about 1×10⁶ cells/mL. 20 cell suspension was added to each well of a 384-well flat-bottom plate followed by incubation at 37° C. in 5% CO₂ incubator overnight.

### 2. Functional Leads Screening by Single Dose

Each BsFp with single dose was used for the functional leads screening, and 0.016 μg/mL (4× stock) VEGF165 protein (GenScript) and 20 μg/mL (4× stock) BsFp (or control) were prepared for functional screening. An anti-VEGF antibody (Avastin®) served as positive control to block VEGF from binding to VEGFR. After adding 10 μL of the 20 μg/mL BsFp (or control) to each well of 384-well plate containing GS-E3/VEGFR2 cells in 20 μL medium (prepared as above in subsection 1), 10 μL of 0.016 μg/mL VEGF165 protein (GenScript) was immediately added into each well, followed by incubation at 37° C. in 5% CO₂ incubator for 6 hours. GS-E3/VEGFR2 cells incubated with VEGF165 protein but without adding any BsFp served as negative control.

### BioGlo™ Luciferase Assay for Assessing VEGF Neutralization

The Bio-Glo™ Luciferase Assay System was used to assess firefly luciferase reporter gene expression from GS-E3/VEGFR2 cells upon VEGF and VEGFR binding. 40 μl Bio-Glo™ (Promega) was added to each well of the 384-well plate containing sample-treated GS-E3/VEGFR2 cells, then incubated for 5 minutes at room temperature and protected from light. The chemiluminescence value (Relative Luminescence Unit, “RLU”) was read on the PHERAstar® microplate reader, data was recorded. A blank well served as detection control.

As can be seen from FIG. 2, in the absence of neutralizing anti-VEGF antibody or VEGF-trap, VEGF (VEGF165) was able to trigger great amount of VEGFR signaling in GS-E3/VEGFR2 cells as reflected by luminescence of the reporter gene. Positive control anti-VEGF antibody (Avastin®) significantly blocked VEGF and VEGFR interaction, thus inhibiting reporter gene expression. All BsFps tested significantly blocked VEGF and VEGFR interaction, resulting in only background level of reporter gene expression (compare to “blank” well). Further, VEGF-trap fusion location did not significantly affect BsFp's ability in neutralizing VEGF. Such VEGF neutralizing activity of BsFps was even stronger than that of the anti-VEGF antibody.

### 3. Functional Leads Bioassay by Dose-Response Curves

0.016 μg/mL (4× stock) VEGF165 protein (GenScript) and 20 μg/mL (4× stock) BsFp (or control) were prepared for dose curve assay. Aflibercept (Eylea®), which shares the same VEGF-trap sequence of the tested BsFps, served as positive control. The first concentration was 20 μg/mL (4× stock) followed by 2.5-fold dilution for the other 7 data points. After adding 10 μL various concentrations of BsFp (or control) dilutions to each well of the 384-well plate containing GS-E3/VEGFR2 cells in 20 μL medium (prepared as above in subsection 1), 10 μL of 0.016 μg/mL VEGF165 protein (GenScript) was immediately added into each well followed by incubation at 37° C. in 5% CO₂ incubator for 6 hours.

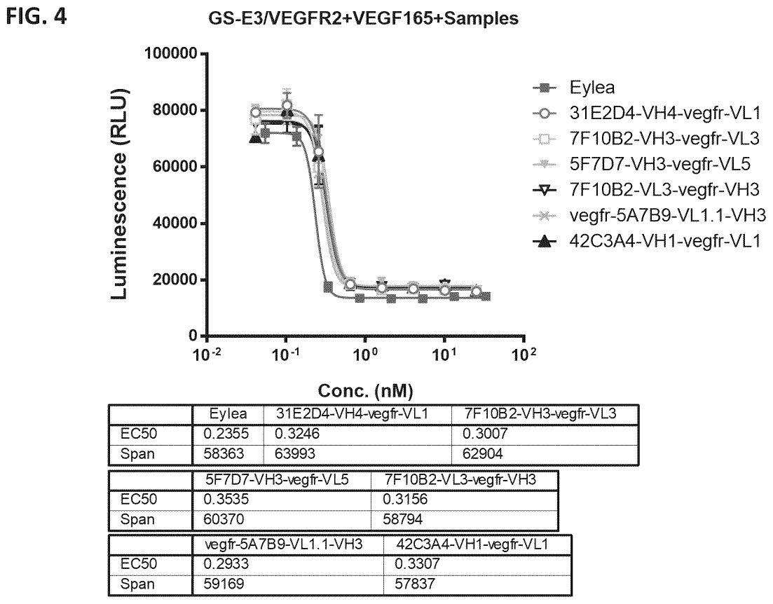

The in vitro VEGF neutralization bioassay was conducted as above using the Bio-Glo™ Luciferase Assay System. As can be seen from FIG. 4, all BsFps tested showed great VEGF neutralization effect comparable to that of the parental VEGF-trap component (Eylea®), regardless of where the VEGF-trap fusion location was.

SEQUENCE LISTING

| VH | VH-CDR1 | VH-CDR2 | VH-CDR3 |
|---|---|---|---|
| 5A7B9-VH3.2 | NYGVN | WINSYSGVPTYADDFKG | GENNYYGGSYD |
| (SEQ ID NO: 82) | (SEQ ID NO: 1) | (SEQ ID NO: 7) | (SEQ ID NO: 12) |
| 5A7B9-VH3 | | | |
| (SEQ ID NO: 83) | | | |
| 5F7D7-VH3 | NYYMH | YIYPNNGDTSYNQKFKG | VSYSNYVAGAMDY |
| (SEQ ID NO: 87) | (SEQ ID NO: 2) | (SEQ ID NO: 8) | (SEQ ID NO: 13) |
| 7F10B2-VH3 | DYNMD | TINPKNGETSDNQKFKA | NVDYSNYLFFPMDY |
| (SEQ ID NO: 89) | (SEQ ID NO: 3) | (SEQ ID NO: 9) | (SEQ ID NO: 14) |
| 7F10B2-VH5 | | | |
| (SEQ ID NO: 90) | | | |
| 31E2D4-VH4 | NYGMN | WINSYSGVPTYADDFKG | GENNYYGGSYD |
| (SEQ ID NO: 93) | (SEQ ID NO: 4) | (SEQ ID NO: 7) | (SEQ ID NO: 12) |
| 42C3A4-VH1 | NYWMD | EIRLKSNNYATHYAESVKG | GAPLFGGYYKGVYFDY |
| (SEQ ID NO: 94) | (SEQ ID NO: 5) | (SEQ ID NO: 10) | (SEQ ID NO: 15) |
| hABTAA-VH | SDYAWN | KISYSGKTDYNPSLKS | GNFEGAMDY |
| (SEQ ID NO: 96) | (SEQ ID NO: 6) | (SEQ ID NO: 11) | (SEQ ID NO: 16) |
| VL | VL-CDR1 | VL-CDR2 | VL-CDR3 |
| 5A7B9-VL1.1 | KASQSVSNDVA | YASNRYT | QQDYSSPLT |
| (SEQ ID NO: 84) | (SEQ ID NO: 17) | (SEQ ID NO: 19) | (SEQ ID NO: 24) |
| 5A7B9-VL1 | | | |
| (SEQ ID NO: 85) | | | |
| 5A7B9-VL2 | | | |
| (SEQ ID NO: 86) | | | |
| 5F7D7-VL5 | KASQSVSNDVA | FASNRYT | QQDYSSPYT |
| (SEQ ID NO: 88) | (SEQ ID NO: 17) | (SEQ ID NO: 20) | (SEQ ID NO: 25) |
| 5F7D7-VL2 | | | |
| (SEQ ID NO: 98) | | | |
| 7F10B2-VL1 | KASQSVSNDVA | YASNRFT | QQDYSSRT |
| (SEQ ID NO: 91) | (SEQ ID NO: 17) | (SEQ ID NO: 21) | (SEQ ID NO: 26) |
| 7F10B2-VL3 | | | |
| (SEQ ID NO: 92) | | | |
| 42C3A4-VL1 | QASQSVSNEVA | YASSRYT | QQDYNSPYT |
| (SEQ ID NO: 95) | (SEQ ID NO: 18) | (SEQ ID NO: 22) | (SEQ ID NO: 27) |
| hABTAA-VL | KASQSVSNDVA | YASNRYP | QQDYSSPWT |
| (SEQ ID NO: 97) | (SEQ ID NO: 17) | (SEQ ID NO: 23) | (SEQ ID NO: 28) |

SEQ ID NO: 29 (signal peptide amino acid sequence)
MGWSCIILFLVATATGVHS
SEQ ID NO: 30 ((G₄S)₃ linker amino acid sequence)
GGGGSGGGGSGGGGS
SEQ ID NO: 31 (E4-linker amino acid sequence)
ESKYGPPSPPSP
SEQ ID NO: 32 (VEGF-trap sequence; Flk1d3 sequence is underlined)
SDTGRPFVEMYSEIPEIIHMTEGRELVIPCRVTSPNITVTLKKFPLDTLIPDGKRIIWDSRKGFIISNATYKEI
GLLTCEATVNGHLYKTNYLTHRQTNTIIDVVLSPSHGIELSVGEKLVLNCTARTELNVGIDFNWEYPSSK
HQHKKLVNRDLKTQSGSEMKKFLSTLTIDGVTRSDQGLYTCAASSGLMTKKNSTFVRVHEK
SEQ ID NO: 33 (5A7B9-VH3.2 heavy chain sequence; CDRs are underlined; Fc fragment is shaded)
QVQLVQSGAEVKKPGASVKVSCKASGYTTFTNYGVNWVRQAPGQRLEWMGWINSYSGVPTYADDFKG

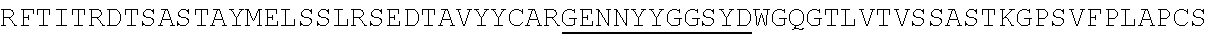

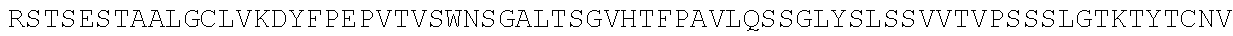

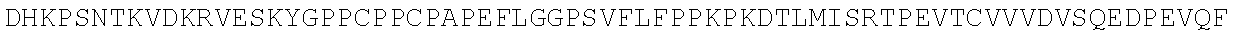

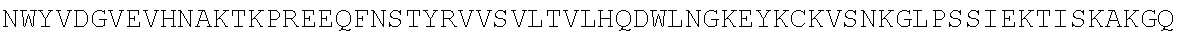

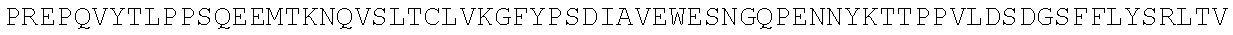

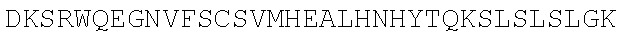

SEQ ID NO: 34 (5A7B9-VH3 heavy chain sequence; CDRs are underlined; Fc fragment is shaded)
QVQLVQSGAEVKKPGASVKVSCKASGYTFTNYGVNWVRQAPGQRLEWMGWINSYSGVPTYADDFKG

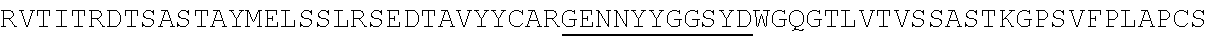

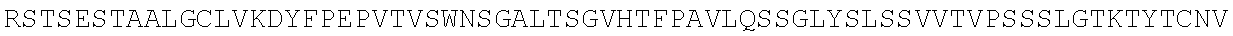

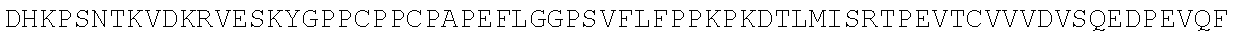

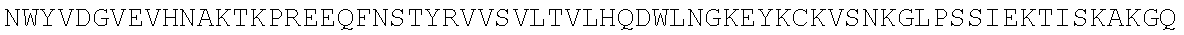

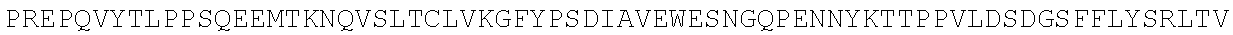

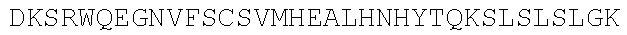

SEQ ID NO: 50 (5A7B9-VH3.2-vegfr heavy chain fusion polypeptide sequence; CDRs are underlined; Fc
fragment is shaded; linker is bolded; VEGF-trap is italicized)
QVQLVQSGAEVKKPGASVKVSCKASGYTFTNYGVNWVRQAPGQRLEWMGWINSYSGVPTYADDFKG

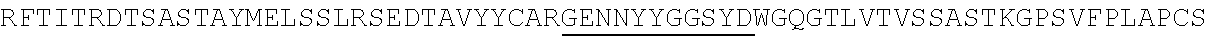

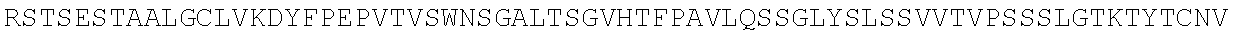

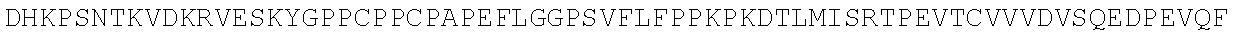

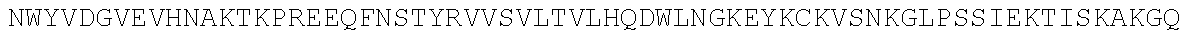

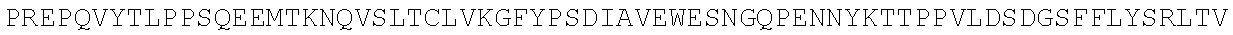

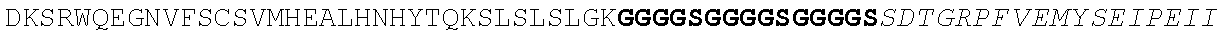

HMTEGRELVIPCRVTSPNITVTLKKFPLDTLIPDGKRIIWDSRKGFIISNATYKEIGLLTCEATVNGHLYKTNYLT
HRQTNTIIDVVLSPSHGIELSVGEKLVLNCTARTELNVGIDFNWEYPSSKHQHKKLVNRDLKTQSGSEMKKFLS
TLTIDGVTRSDQGLYTCAASSGLMTKKNSTFVRVHEK
SEQ ID NO: 51 (5A7B9-VH3-vegfr heavy chain fusion polypeptide sequence; CDRs are underlined; Fc
fragment is shaded; linker is bolded; VEGF-trap is italicized)
QVQLVQSGAEVKKPGASVKVSCKASGYTFTNYGVNWVRQAPGQRLEWMGWINSYSGVPTYADDFKG

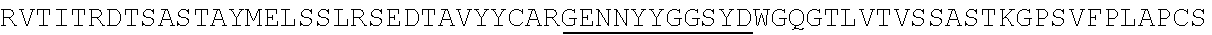

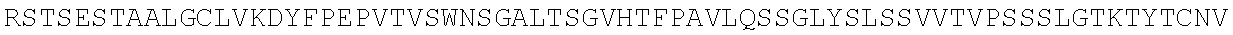

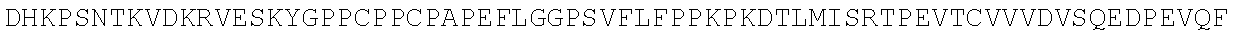

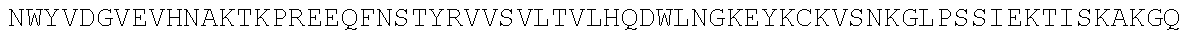

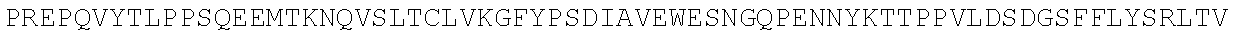

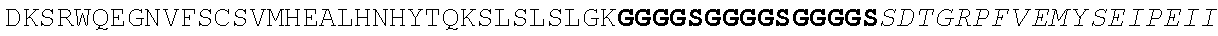

HMTEGRELVIPCRVTSPNITVTLKKFPLDTLIPDGKRIIWDSRKGFIISNATYKEIGLLTCEATVNGHLYKTNYLT
HRQTNTIIDVVLSPSHGIELSVGEKLVLNCTARTELNVGIDFNWEYPSSKHQHKKLVNRDLKTQSGSEMKKFLS
TLTIDGVTRSDQGLYTCAASSGLMTKKNSTFVRVHEK
SEQ ID NO: 41 (5A7B9-VL1.1 light chain sequence; CDRs are underlined; CL is shaded)
DIQMTQSPSSLSASVGDRVTITCKASQSVSNDVAWYQQKPGKAPKLLIYYASNRYTGVPSRFSGSGSGT

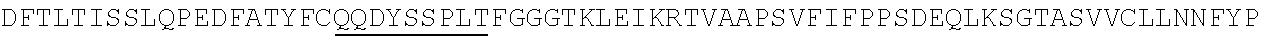

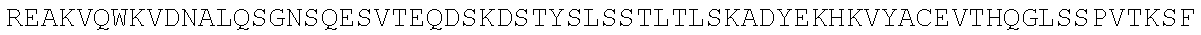

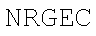

SEQ ID NO: 42 (5A7B9-VL1 light chain sequence; CDRs are underlined; CL is shaded)
DIQMTQSPSSLSASVGDRVTITCKASQSVSNDVAWYQQKPGKAPKLLIYYASNRYTGVPSRFSGSGSGT

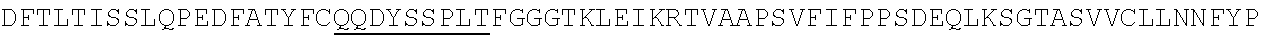

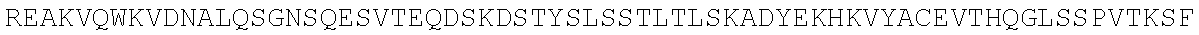

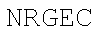

SEQ ID NO: 43 (5A7B9-VL2 light chain sequence; CDRs are underlined; CL is shaded)
DIQLTQSPSSLSASVGDRVTITCKASQSVSNDVAWYQQKPGKAPKLLIYYASNRYTGVPSRFSGSGSGTD

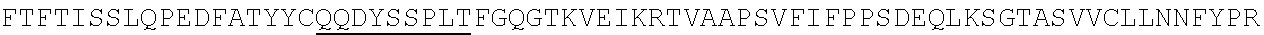

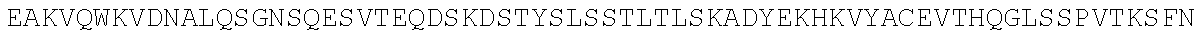

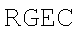

SEQ ID NO: 63 (5A7B9-VL1.1-vegfr light chain fusion polypeptide sequence; CDRs are underlined; CL
is shaded; linker is bolded; VEGF-trap is italicized)
DIQMTQSPSSLSASVGDRVTITCKASQSVSNDVAWYQQKPGKAPKLLIYYASNRYTGVPSRFSGSGSGT

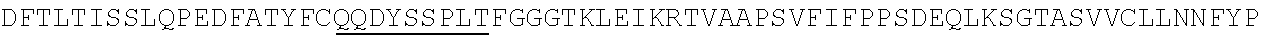

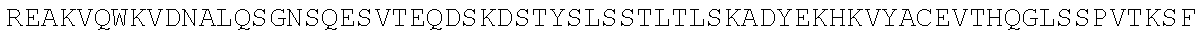

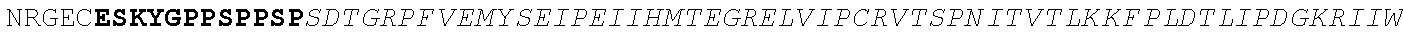

DSRKGFIISNATYKEIGLLTCEATVNGHLYKTNYLTHRQTNTIIDVVLSPSHGIELSVGEKLVLNCTARTELNVGI
DFNWEYPSSKHQHKKLVNRDLKTQSGSEMKKFLSTLTIDGVTRSDQGLYTCAASSGLMTKKNSTFVRVHEK
SEQ ID NO: 62 (5A7B9-VL1-vegfr light chain fusion polypeptide sequence; CDRs are underlined; CL is
shaded; linker is bolded; VEGF-trap is italicized)
DIQMTQSPSSLSASVGDRVTITCKASQSVSNDVAWYQQKPGKAPKLLIYYASNRYTGVPSRFSGSGSGT

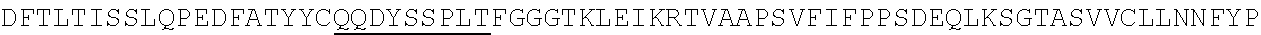

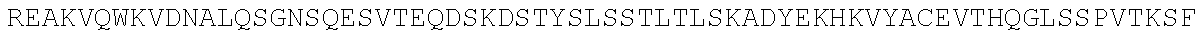

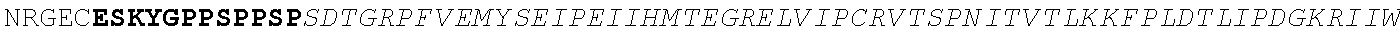

DSRKGFIISNATYKEIGLLTCEATVNGHLYKTNYLTHRQTNTIIDVVLSPSHGIELSVGEKLVLNCTARTELNVGI
DFNWEYPSSKHQHKKLVNRDLKTQSGSEMKKFLSTLTIDGVTRSDQGLYTCAASSGLMTKKNSTFVRVHEK
SEQ ID NO: 76 (5A7B9-VL2-vegfr light chain fusion polypeptide sequence; CDRs are underlined; CL is
shaded; linker is bolded; VEGF-trap is italicized)
DIQLTQSPSSLSASVGDRVTITCKASQSVSNDVAWYQQKPGKAPKLLIYYASNRYTGVPSRFSGSGSGTD

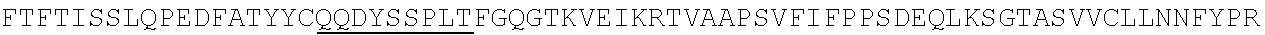

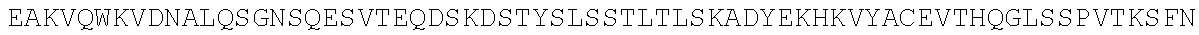

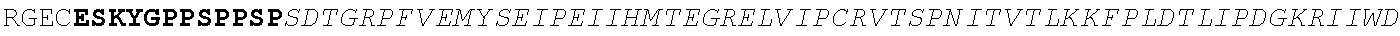

SRKGFIISNATYKEIGLLTCEATVNGHLYKTNYLTHRQTNTIIDVVLSPSHGIELSVGEKLVLNCTARTELNVGID
FNWEYPSSKHQHKKLVNRDLKTQSGSEMKKFLSTLTIDGVTRSDQGLYTCAASSGLMTKKNSTFVRVHEK
SEQ ID NO: 35 (5F7D7-VH3 heavy chain sequence; CDRs are underlined; Fc fragment is shaded)
QVQLVQSGAEVKKPGASVKVSCKASGYTFTNYYMHWVRQAPGQRLEWIGYIYPNNGDTSYNQKFKG

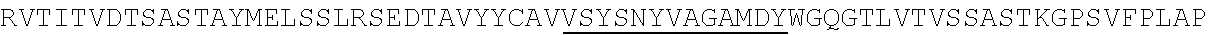

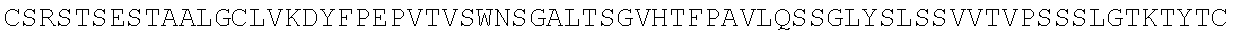

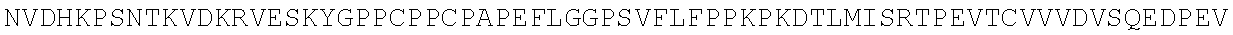

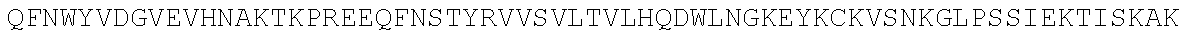

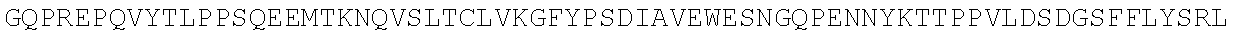

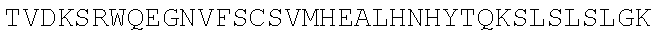

SEQ ID NO: 44 (5F7D7-VL5 light chain sequence; CDRs are underlined; CL is shaded)
DIQMTQSPSSLSASVGDRVTITCKASQSVSNDVAWYQQKPGKAPKLLIYFASNRYTGVPSRFSGSGYGT

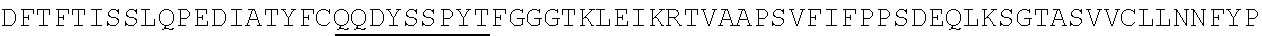

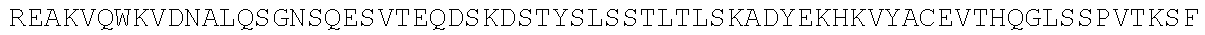

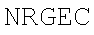

SEQ ID NO: 49 (5F7D7-VL2 light chain sequence; CDRs are underlined; CL is shaded)
DIQLTQSPSSLSASVGDRVTITCKASQSVSNDVAWYQQKPGKAPKLLIYFASNRYTGVPSRFSGSGSGTD

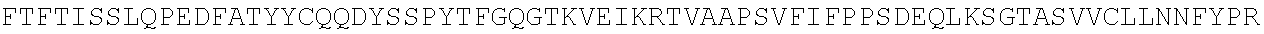

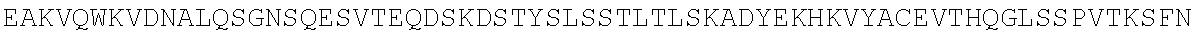

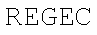

SEQ ID NO: 55 (5F7D7-VH3-vegfr heavy chain fusion polypeptide sequence; CDRs are underlined; Fc
fragment is shaded; linker is bolded; VEGF-trap is italicized)
QVQLVQSGAEVKKPGASVKVSCKASGYTFTNYYMHWVRQAPGQRLEWIGYIYPNNGDTSYNQKFKG

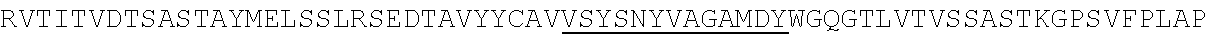

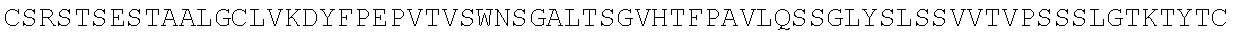

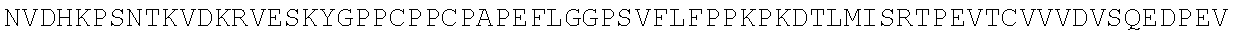

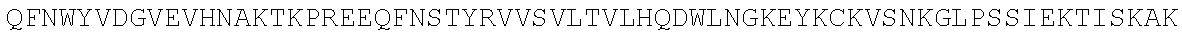

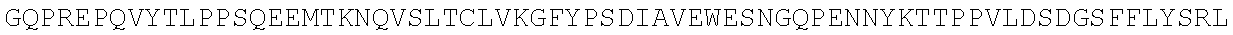

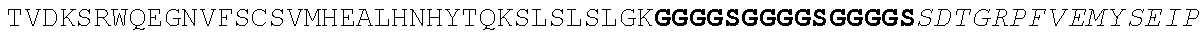

EIIHMTEGRELVIPCRVTSPNITVTLKKFPLDTLIPDGKRIIWDSRKGFIISNATYKEIGLLTCEATVNGHLYKTNY
LTHRQTNTIIDVVLSPSHGIELSVGEKLVLNCTARTELNVGIDENWEYPSSKHQHKKLVNRDLKTQSGSEMKKF
LSTLTIDGVTRSDQGLYTCAASSGLMTKKNSTFVRVHEK
SEQ ID NO: 66 (5F7D7-VL5-vegfr light chain fusion polypeptide sequence; CDRs are underlined; CL is
shaded; linker is bolded; VEGF-trap is italicized)
DIQMTQSPSSLSASVGDRVTITCKASQSVSNDVAWYQQKPGKAPKLLIYFASNRYTGVPSRFSGSGYGT

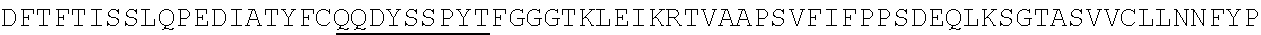

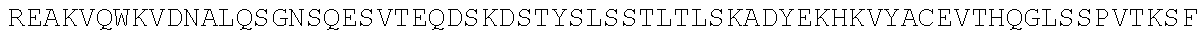

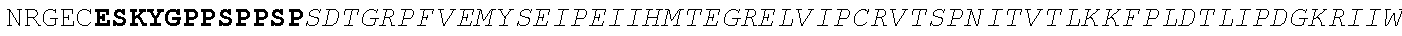

DSRKGFIISNATYKEIGLLTCEATVNGHLYKTNYLTHRQTNTIIDVVLSPSHGIELSVGEKLVLNCTARTELNVGI
DFNWEYPSSKHQHKKLVNRDLKTQSGSEMKKFLSTLTIDGVTRSDQGLYTCAASSGLMTKKNSTFVRVHEK
SEQ ID NO: 36 (7F10B2-VH3 heavy chain sequence; CDRs are underlined; Fc fragment is shaded)
QVQLVQSGAEVKKPGASVKVSCKASGYTFTDYNMDWVRQAPGQGLEWMGTINPKNGETSDNQKFK

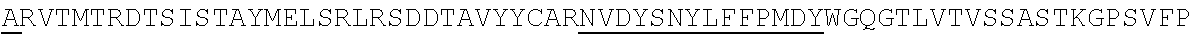

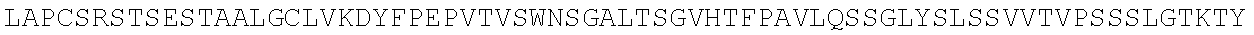

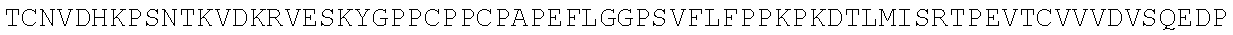

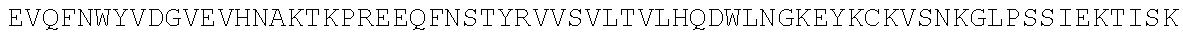

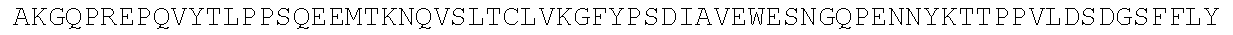

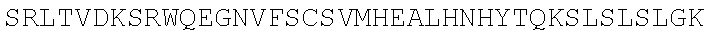

SEQ ID NO: 37 (7F10B2-VH5 heavy chain sequence; CDRs are underlined; Fc fragment is shaded)
QVQLVQSGAEVKKPGASVKVSCKASGYTFTDYNMDWVRQAPGQGLEWMGTINPKNGETSDNQKFK

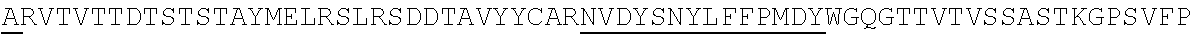

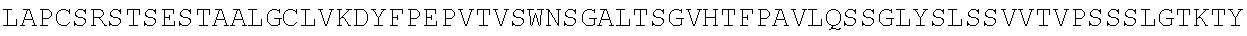

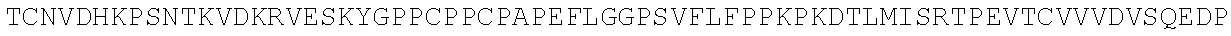

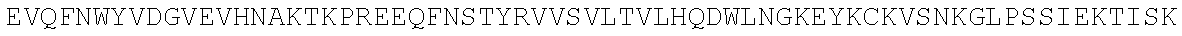

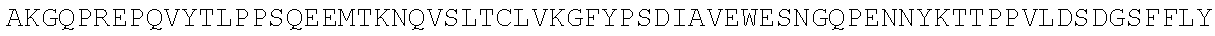

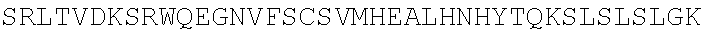

SEQ ID NO: 54 (7F10B2-VH3-vegfr heavy chain fusion polypeptide sequence; CDRs are underlined; Fc
fragment is shaded; linker is bolded; VEGF-trap is italicized)
QVQLVQSGAEVKKPGASVKVSCKASGYTFTDYNMDWVRQAPGQGLEWMGTINPKNGETSDNQKFK

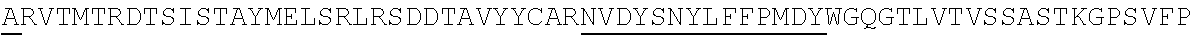

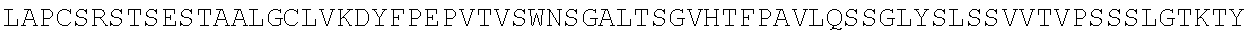

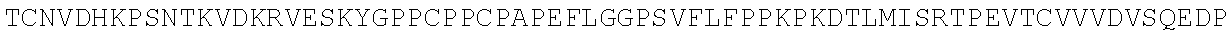

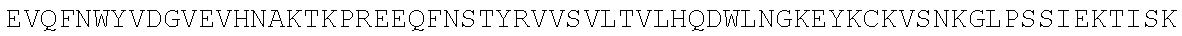

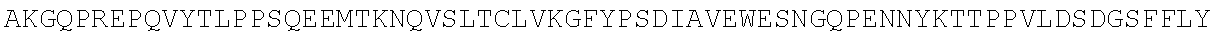

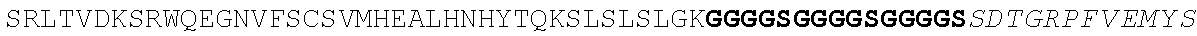

EIPEIIHMTEGRELVIPCRVTSPNITVTLKKFPLDTLIPDGKRIIWDSRKGFIISNATYKEIGLLTCEATVNGHLYK
TNYLTHRQTNTIIDVVLSPSHGIELSVGEKLVLNCTARTELNVGIDENWEYPSSKHQHKKLVNRDLKTQSGSEM
KKFLSTLTIDGVTRSDQGLYTCAASSGLMTKKNSTFVRVHEK
SEQ ID NO: 53 (7F10B2-VH5-vegfr heavy chain fusion polypeptide sequence; CDRs are underlined; Fc
fragment is shaded; linker is bolded; VEGF-trap is italicized)
QVQLVQSGAEVKKPGASVKVSCKASGYTFTDYNMDWVRQAPGQGLEWMGTINPKNGETSDNQKFK

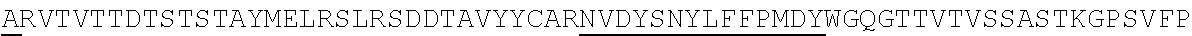

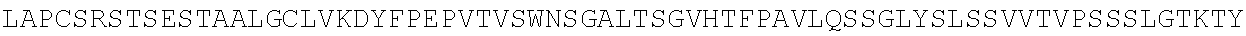

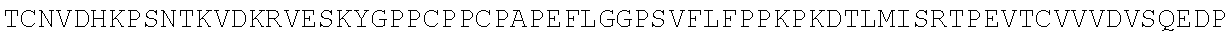

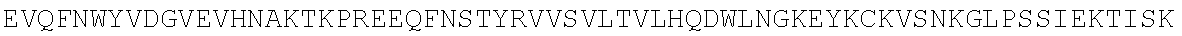

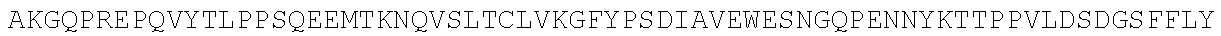

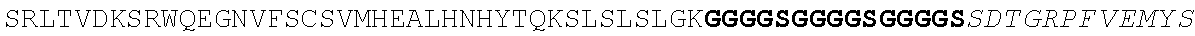

EIPEIIHMTEGRELVIPCRVTSPNITVTLKKFPLDTLIPDGKRIIWDSRKGFIISNATYKEIGLLTCEATVNGHLYK
TNYLTHRQTNTIIDVVLSPSHGIELSVGEKLVLNCTARTELNVGIDENWEYPSSKHQHKKLVNRDLKTQSGSEM
KKFLSTLTIDGVTRSDQGLYTCAASSGLMTKKNSTFVRVHEK
SEQ ID NO: 45 (7F10B2-VL1 light chain sequence; CDRs are underlined; CL is shaded)
DIQMTQSPSSLSASVGDRVTITCKASQSVSNDVAWYQQKPGKAPKLLIYYASNRFTGVPSRFSGSGSGTD

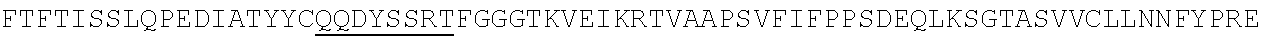

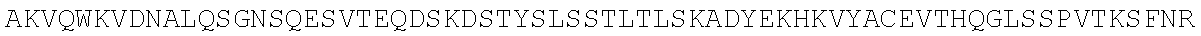

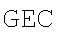

SEQ ID NO: 46 (7F10B2-VL3 light chain sequence; CDRs are underlined; CL is shaded)
DIQMTQSPSSLSASVGDRVTITCKASQSVSNDVAWYQQKPGKAPKLLIYYASNRFTGVPSRFSGSGSGTD

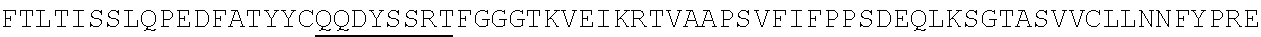

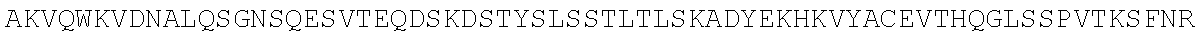

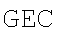

SEQ ID NO: 64 (7F10B2-VL1-vegfr light chain fusion polypeptide sequence; CDRs are underlined; CL is
shaded; linker is bolded; VEGF-trap is italicized)
DIQMTQSPSSLSASVGDRVTITCKASQSVSNDVAWYQQKPGKAPKLLIYYASNRFTGVPSRFSGSGSGTD

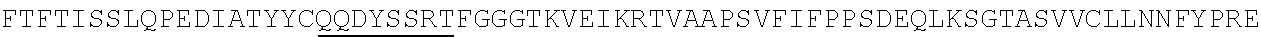

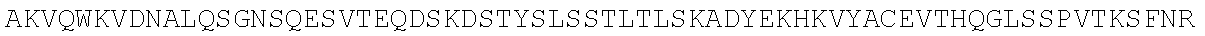

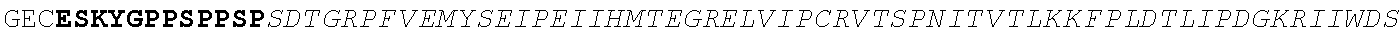

RKGFIISNATYKEIGLLTCEATVNGHLYKTNYLTHRQTNTIIDVVLSPSHGIELSVGEKLVLNCTARTELNVGIDF
NWEYPSSKHQHKKLVNRDLKTQSGSEMKKFLSTLTIDGVTRSDQGLYTCAASSGLMTKKNSTFVRVHEK
SEQ ID NO: 65 (7F10B2-VL3-vegfr light chain fusion polypeptide sequence; CDRs are underlined; CL is
shaded; linker is bolded; VEGF-trap is italicized)
DIQMTQSPSSLSASVGDRVTITCKASQSVSNDVAWYQQKPGKAPKLLIYYASNRFTGVPSRFSGSGSGTD

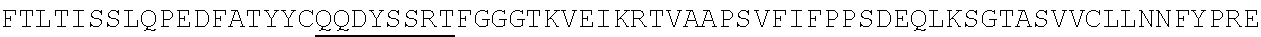

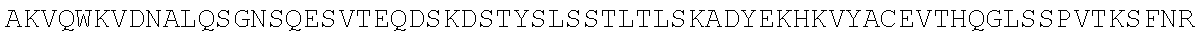

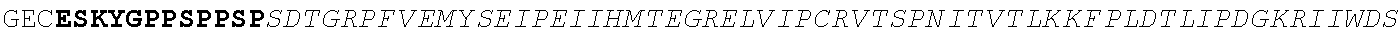

RKGFIISNATYKEIGLLTCEATVNGHLYKTNYLTHRQTNTIIDVVLSPSHGIELSVGEKLVLNCTARTELNVGIDF
NWEYPSSKHQHKKLVNRDLKTQSGSEMKKFLSTLTIDGVTRSDQGLYTCAASSGLMTKKNSTFVRVHEK
SEQ ID NO: 38 (31E2D4-VH4 heavy chain sequence; CDRs are underlined; Fc fragment is shaded)
QVQLVQSGAEVKKPGASVKVSCKASGYTLTNYGMNWVRQATGQGLEWMGWINSYSGVPTYADDFK

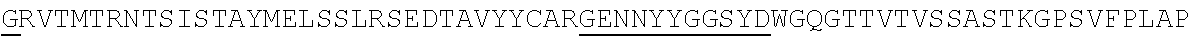

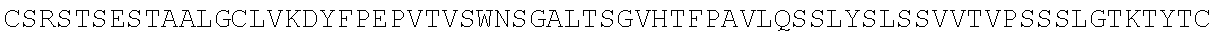

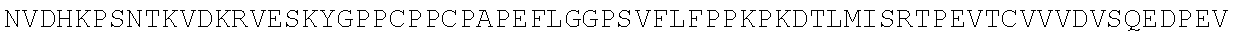

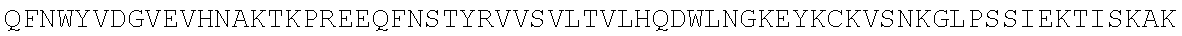

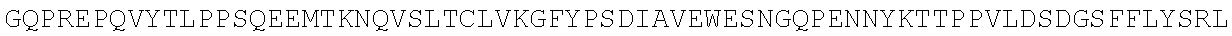

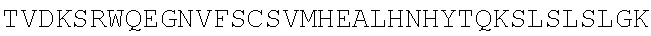

SEQ ID NO: 52 (31E2D4-VH4-vegfr heavy chain fusion polypeptide sequence; CDRs are underlined; Fc
fragment is shaded; linker is bolded; VEGF-trap is italicized)
QVQLVQSGAEVKKPGASVKVSCKASGYTLTNYGMNWVRQATGQGLEWMGWINSYSGVPTYADDFK

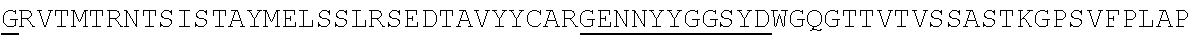

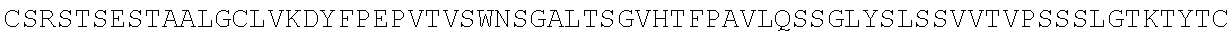

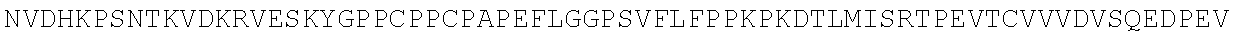

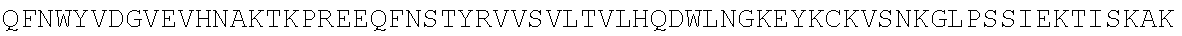

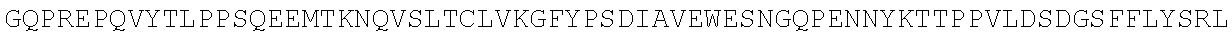

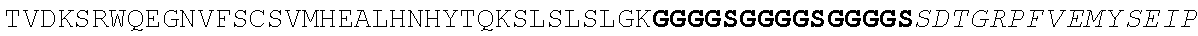

EIIHMTEGRELVIPCRVTSPNITVTLKKFPLDTLIPDGKRIIWDSRKGFIISNATYKEIGLLTCEATVNGHLYKTNY
LTHRQTNTIIDVVLSPSHGIELSVGEKLVLNCTARTELNVGIDENWEYPSSKHQHKKLVNRDLKTQSGSEMKKF
LSTLTIDGVTRSDQGLYTCAASSGLMTKKNSTFVRVHEK
SEQ ID NO: 39 (42C3A4-VH1 heavy chain sequence; CDRs are underlined; Fc fragment is shaded)
EVQLVESGGGLVQPGGSLKVSCAASGITFSNYWMDWVRQASGKGLEWVGEIRLKSNNYATHYAESVK

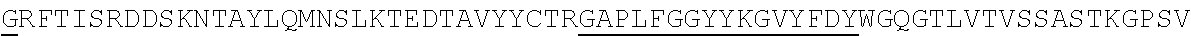

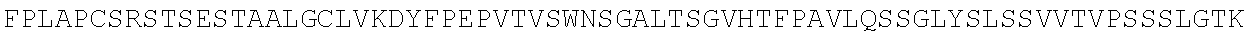

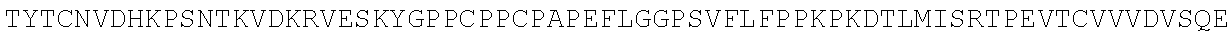

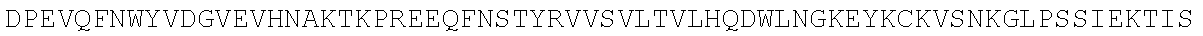

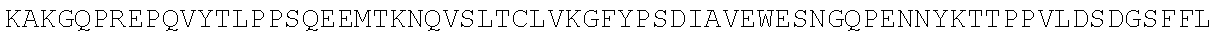

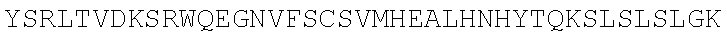

SEQ ID NO: 74 (42C3A4-VH1-vegfr heavy chain fusion polypeptide sequence; CDRs are underlined; Fc
fragment is shaded; linker is bolded; VEGF-trap is italicized)
EVQLVESGGGLVQPGGSLKVSCAASGITFSNYWMDWVRQASGKGLEWVGEIRLKSNNYATHYAESVK

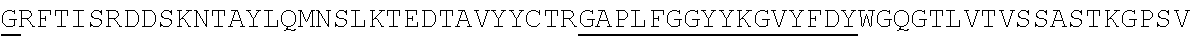

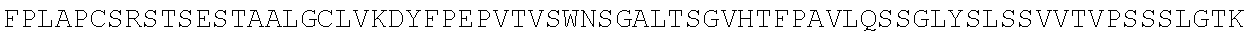

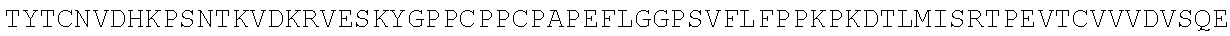

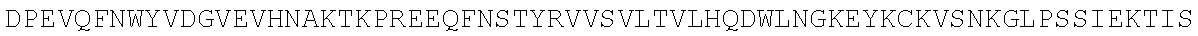

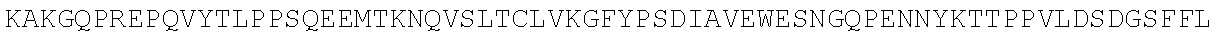

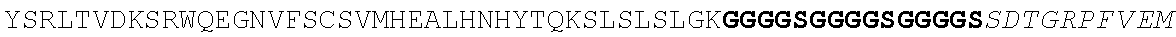

YSEIPEIIHMTEGRELVIPCRVTSPNITVTLKKFPLDTLIPDGKRIIWDSRKGFIISNATYKEIGLLTCEATVNGHLY
KTNYLTHRQTNTIIDVVLSPSHGIELSVGEKLVLNCTARTELNVGIDENWEYPSSKHQHKKLVNRDLKTQSGSE
MKKFLSTLTIDGVTRSDQGLYTCAASSGLMTKKNSTFVRVHEK
SEQ ID NO: 47 (42C3A4-VL1 light chain sequence; CDRs are underlined; CL is shaded)
DIQMTQSPSSLSASVGDRVTITCQASQSVSNEVAWYQQKPGKAPKLLIYYASSRYTGVPSRFSGSGSGTD

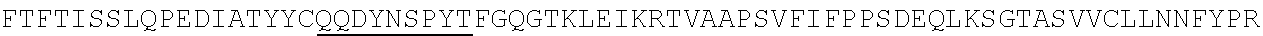

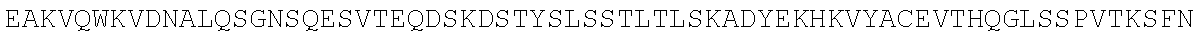

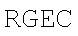

SEQ ID NO: 72 (42C3A4-VL1-vegfr light chain fusion polypeptide sequence; CDRs are underlined; CL is
shaded; linker is bolded; VEGF-trap is italicized)
DIQMTQSPSSLSASVGDRVTITCQASQSVSNEVAWYQQKPGKAPKLLIYYASSRYTGVPSRFSGSGSGTD

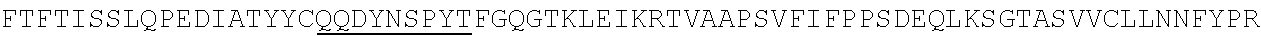

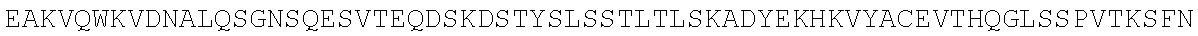

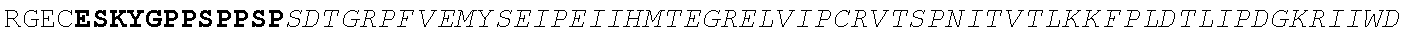

SRKGFIISNATYKEIGLLTCEATVNGHLYKTNYLTHRQTNTIIDVVLSPSHGIELSVGEKLVLNCTARTELNVGID
FNWEYPSSKHQHKKLVNRDLKTQSGSEMKKFLSTLTIDGVTRSDQGLYTCAASSGLMTKKNSTFVRVHEK
SEQ ID NO: 40 (hABTAA-VH heavy chain sequence; CDRs are underlined; Fc fragment is shaded)
QVQLQESGPGLVKPSETLSLTCAVSGYSITSDYAWNWIRQPPGKGLEWMGKISYSGKTDYNPSLKSRSTI

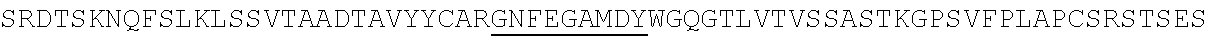

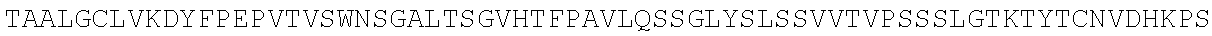

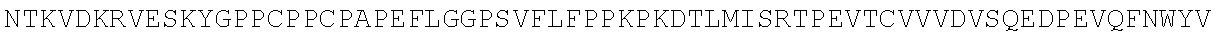

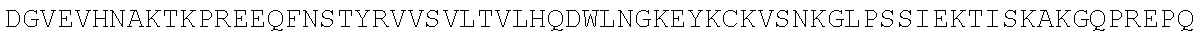

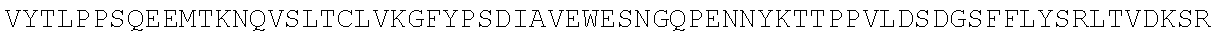

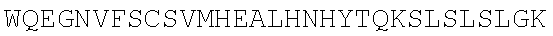

SEQ ID NO: 78 (hABTAA-VH-vegfr heavy chain fusion polypeptide sequence; CDRs are underlined; Fc
fragment is shaded; linker is bolded; VEGF-trap is italicized)
QVQLQESGPGLVKPSETLSLTCAVSGYSITSDYAWNWIRQPPGKGLEWMGKISYSGKTDYNPSLKSRSTI

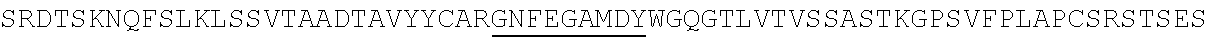

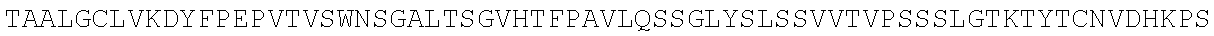

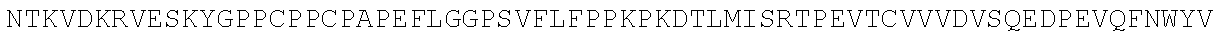

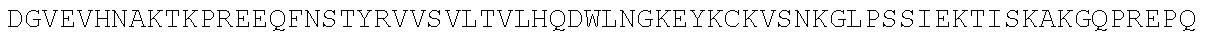

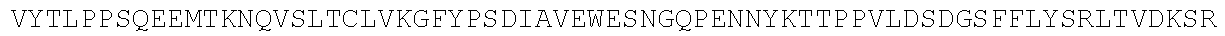

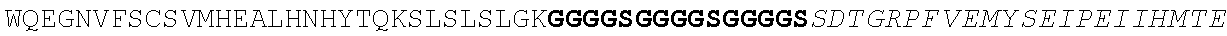

GRELVIPCRVTSPNITVTLKKFPLDTLIPDGKRIIWDSRKGFIISNATYKEIGLLTCEATVNGHLYKTNYLTHRQT
NTIIDVVLSPSHGIELSVGEKLVLNCTARTELNVGIDENWEYPSSKHQHKKLVNRDLKTQSGSEMKKFLSTLTID
GVTRSDQGLYTCAASSGLMTKKNSTFVRVHEK
SEQ ID NO: 48 (hABTAA-VL light chain sequence; CDRs are underlined; CL is shaded)
DIQMTQSPSSLSASVGDRVTITCKASQSVSNDVAWYQQKPGKAPKLLIYYASNRYPGVPSRFSGSGSGT

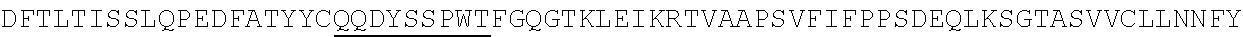

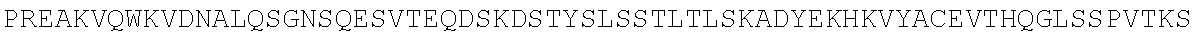

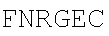

SEQ ID NO: 80 (hABTAA-VL-vegfr light chain fusion polypeptide sequence; CDRs are underlined; CL is
shaded; linker is bolded; VEGF-trap is italicized)
DIQMTQSPSSLSASVGDRVTITCKASQSVSNDVAWYQQKPGKAPKLLIYYASNRYPGVPSRFSGSGSGT

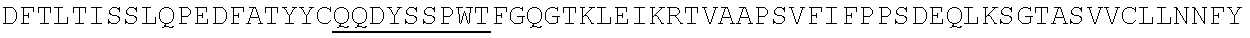

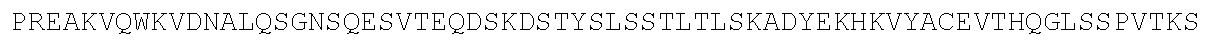

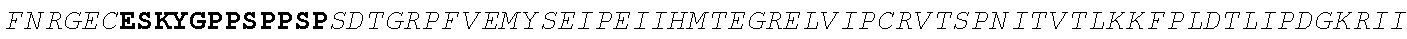

WDSRKGFIISNATYKEIGLLTCEATVNGHLYKTNYLTHRQTNTIIDVVLSPSHGIELSVGEKLVLNCTARTELNVG
IDFNWEYPSSKHQHKKLVNRDLKTQSGSEMKKFLSTLTIDGVTRSDQGLYTCAASSGLMTKKNSTFVRVHEK
SEQ ID NO: 56 (vegfr-5A7B9-VH3.2 heavy chain fusion polypeptide sequence; CDRs are underlined; Fc
fragment is shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSQ
VQLVQSGAEVKKPGASVKVSCKASGYTFTNYGVNWVRQAPGQRLEWMGWINSYSGVPTYADDFKGR

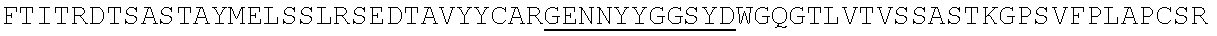

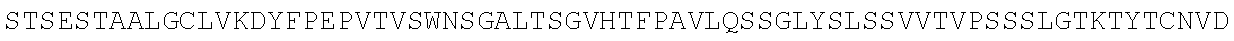

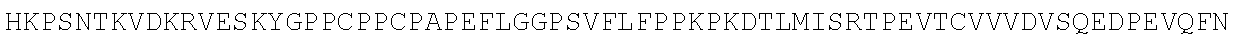

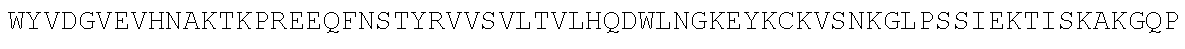

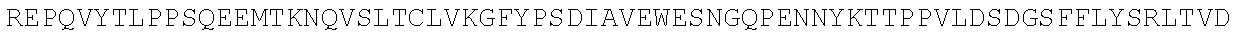

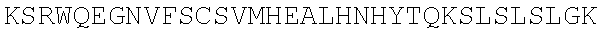

SEQ ID NO: 57 (vegfr-5A7B9-VH3 heavy chain fusion polypeptide sequence; CDRs are underlined; Fc
fragment is shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSQ
VQLVQSGAEVKKPGASVKVSCKASGYTFTNYGVNWVRQAPGQRLEWMGWINSYSGVPTYADDFKGR

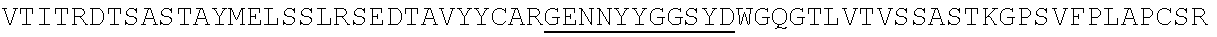

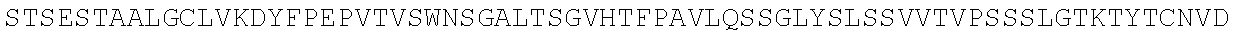

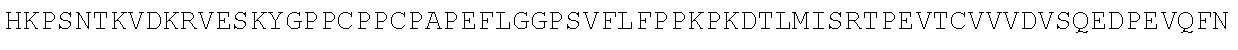

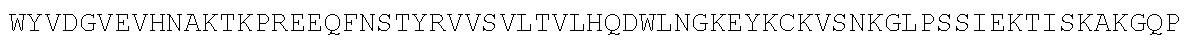

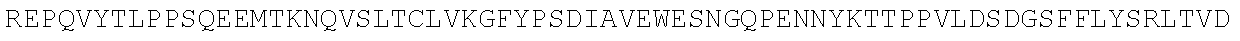

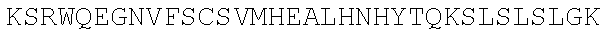

SEQ ID NO: 68 (vegfr-5A7B9-VL1.1 light chain fusion polypeptide sequence; CDRs are underlined; CL
is shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSD
IQMTQSPSSLSASVGDRVTITCKASQSVSNDVAWYQQKPGKAPKLLIYYASNRYTGVPSRFSGSGSGTDF

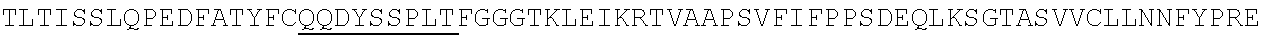

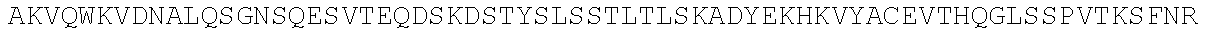

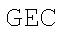

SEQ ID NO: 67 (vegfr-5A7B9-VL1 light chain fusion polypeptide sequence; CDRs are underlined; CL is
shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSD
IQMTQSPSSLSASVGDRVTITCKASQSVSNDVAWYQQKPGKAPKLLIYYASNRYTGVPSRFSGSGSGTDF

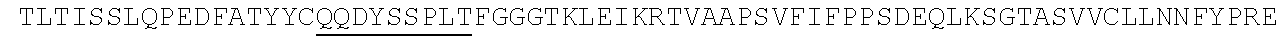

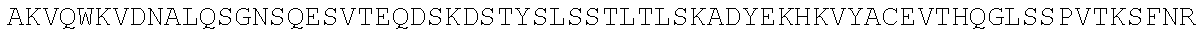

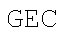

SEQ ID NO: 77 (vegfr-5A7B9-VL2 light chain fusion polypeptide sequence; CDRs are underlined; CL is
shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSD
IQLTQSPSSLSASVGDRVTITCKASQSVSNDVAWYQQKPGKAPKLLIYYASNRYTGVPSRFSGSGSGTDF

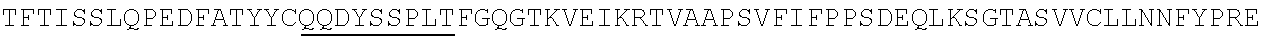

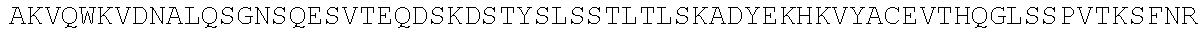

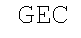

SEQ ID NO: 61 (vegfr-5F7D7-VH3 heavy chain fusion polypeptide sequence; CDRs are underlined; Fc
fragment is shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSQ
VQLVQSGAEVKKPGASVKVSCKASGYTFTNYYMHWVRQAPGQRLEWIGYIYPNNGDTSYNQKFKGR

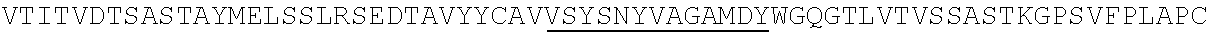

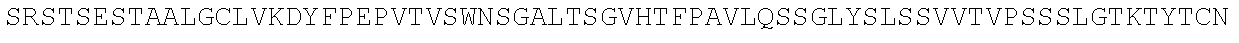

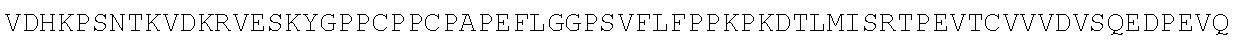

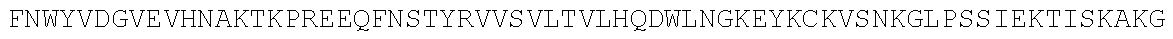

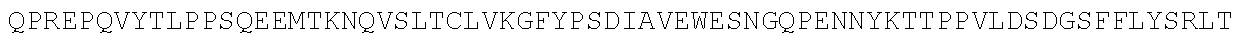

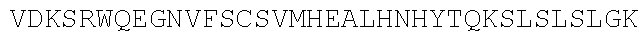

SEQ ID NO: 71 (vegfr-5F7D7-VL5 light chain fusion polypeptide sequence; CDRs are underlined; CL is
shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSD
IQMTQSPSSLSASVGDRVTITCKASQSVSNDVAWYQQKPGKAPKLLIYFASNRYTGVPSRFSGSGYGTDF

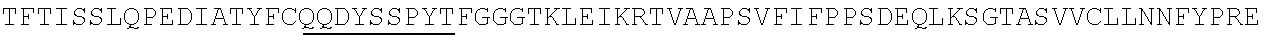

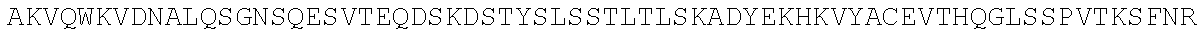

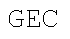

SEQ ID NO: 60 (vegfr-7F10B2-VH3 heavy chain fusion polypeptide sequence; CDRs are underlined; Fc
fragment is shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSQ
VQLVQSGAEVKKPGASVKVSCKASGYTFTDYNMDWVRQAPGQGLEWMGTINPKNGETSDNQKFKAR

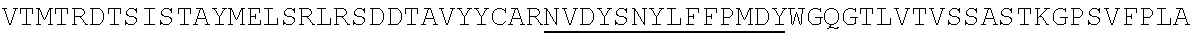

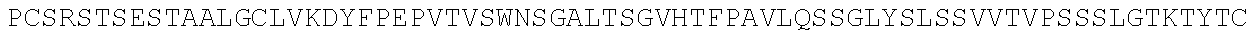

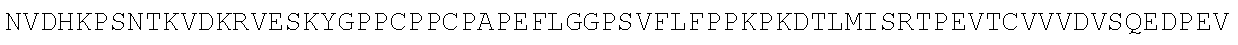

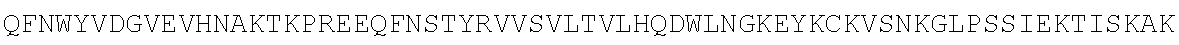

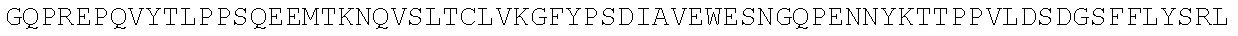

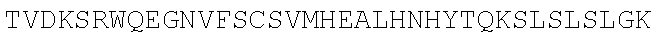

SEQ ID NO: 59 (vegfr-7F10B2-VH5 heavy chain fusion polypeptide sequence; CDRs are underlined; Fc
fragment is shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSQ
VQLVQSGAEVKKPGASVKVSCKASGYTFTDYNMDWVRQAPGQGLEWMGTINPKNGETSDNQKFKAR

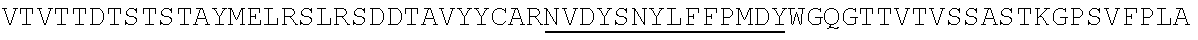

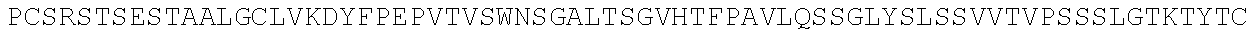

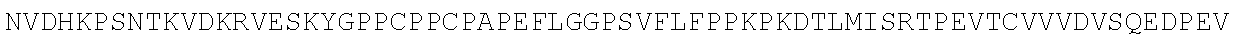

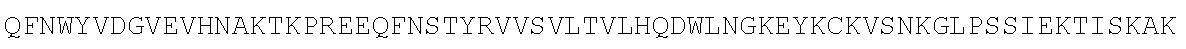

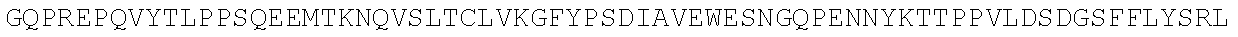

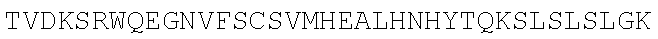

SEQ ID NO: 69 (vegfr-7F10B2-VL1 light chain fusion polypeptide sequence; CDRs are underlined; CL is
shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSD
IQMTQSPSSLSASVGDRVTITCKASQSVSNDVAWYQQKPGKAPKLLIYYASNRFTGVPSRFSGSGSGTDF

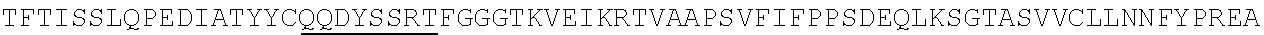

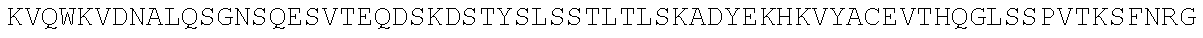

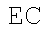

SEQ ID NO: 70 (vegfr-7F10B2-VL3 light chain fusion polypeptide sequence; CDRs are underlined; CL is
shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLV
NRDLKTQSGSEMKKFLSTLTIDGVTRSDQGLYTCAASSGLMTKKNSTFVRVHEKGGGGSGGGGSGGGGSD
IQMTQSPSSLSASVGDRVTITCKASQSVSNDVAWYQQKPGKAPKLLIYYASNRFTGVPSRFSGSGSGTDF

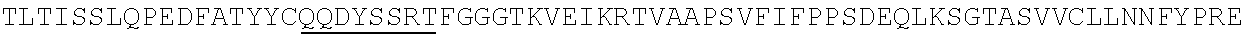

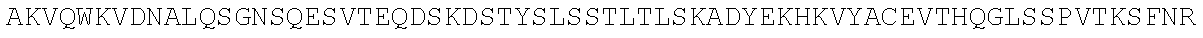

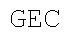

SEQ ID NO: 58 (vegfr-31E2D4-VH4 heavy chain fusion polypeptide sequence; CDRs are underlined; Fc
fragment is shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSQ
VQLVQSGAEVKKPGASVKVSCKASGYTLTNYGMNWVRQATGQGLEWMGWINSYSGVPTYADDFKGR

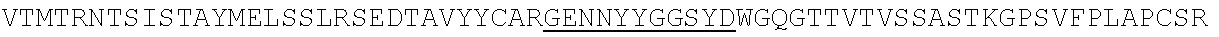

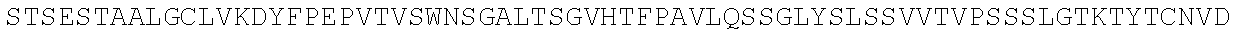

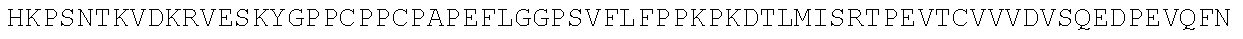

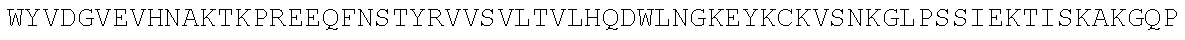

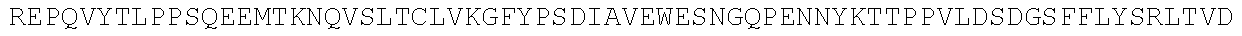

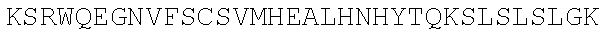

SEQ ID NO: 75 (vegfr-42C3A4-VH1 heavy chain fusion polypeptide sequence; CDRs are underlined; Fc
fragment is shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSE
VQLVESGGGLVQPGGSLKVSCAASGITFSNYWMDWVRQASGKGLEWVGEIRLKSNNYATHYAESVKG

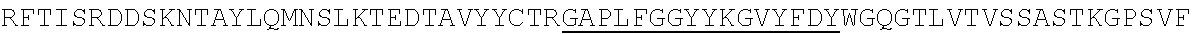

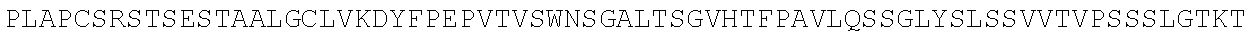

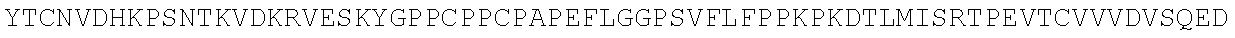

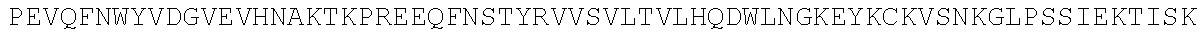

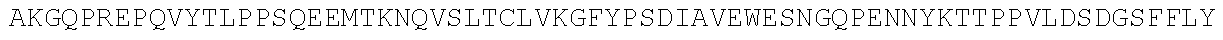

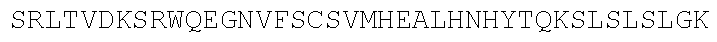

SEQ ID NO: 73 (vegfr-42C3A4-VL1 light chain fusion polypeptide sequence; CDRs are underlined; CL is
shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSD
IQMTQSPSSLSASVGDRVTITCQASQSVSNEVAWYQQKPGKAPKLLIYYASSRYTGVPSRFSGSGSGTDF

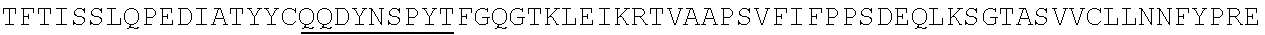

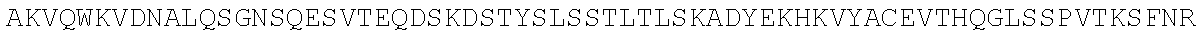

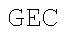

SEQ ID NO: 79 (vegfr-hABTAA-VH heavy chain fusion polypeptide sequence; CDRs are underlined; Fc
fragment is shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSQ
VQLQESGPGLVKPSETLSLTCAVSGYSITSDYAWNWIRQPPGKGLEWMGKISYSGKTDYNPSLKSRSTIS

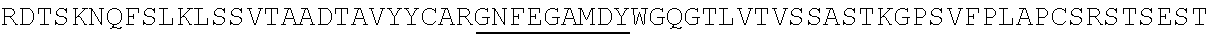

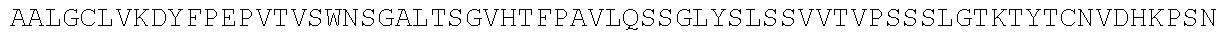

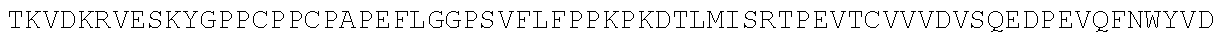

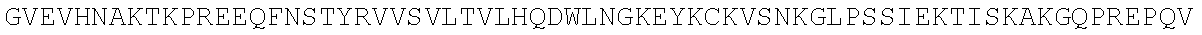

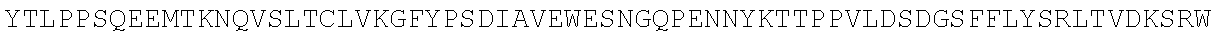

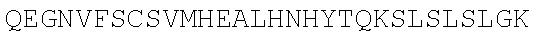

SEQ ID NO: 81 (vegfr-hABTAA-VL light chain fusion polypeptide sequence; CDRs are underlined; CL is
shaded; linker is bolded; VEGF-trap is italicized)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLV
NRDLKTQSGSEMKKFLSTLTIDGVTRSDQGLYTCAASSGLMTKKNSTFVRVHEKGGGGSGGGGSGGGGSD
IQMTQSPSSLSASVGDRVTITCKASQSVSNDVAWYQQKPGKAPKLLIYYASNRYPGVPSRFSGSGSGTDF

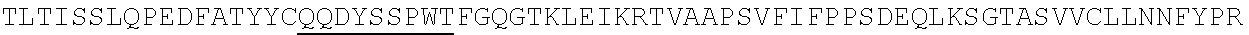

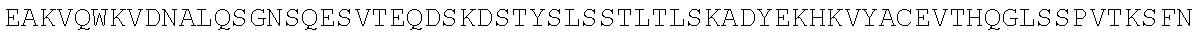

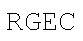

SEQ ID NO: 82 (5A7B9-VH3.2 VH sequence; CDRs are underlined)
QVQLVQSGAEVKKPGASVKVSCKASGYTFTNYGVNWVRQAPGQRLEWMGWINSYSGVPTYADDFKG
RFTITRDTSASTAYMELSSLRSEDTAVYYCARGENNYYGGSYDWGQGTLVTVSS
SEQ ID NO: 83 (5A7B9-VH3 VH sequence; CDRs are underlined)
QVQLVQSGAEVKKPGASVKVSCKASGYTFTNYGVNWVRQAPGQRLEWMGWINSYSGVPTYADDFKG
RVTITRDTSASTAYMELSSLRSEDTAVYYCARGENNYYGGSYDWGQGTLVTVSS
SEQ ID NO: 84 (5A7B9-VL1.1 VL sequence; CDRs are underlined)
DIQMTQSPSSLSASVGDRVTITCKASQSVSNDVAWYQQKPGKAPKLLIYYASNRYTGVPSRFSGSGSGT
DFTLTISSLQPEDFATYFCQQDYSSPLTFGGGTKLEIK
SEQ ID NO: 85 (5A7B9-VL1 VL sequence; CDRs are underlined)
DIQMTQSPSSLSASVGDRVTITCKASQSVSNDVAWYQQKPGKAPKLLIYYASNRYTGVPSRFSGSGSGT
DFTLTISSLQPEDFATYYCQQDYSSPLTFGGGTKLEIK
SEQ ID NO: 86 (5A7B9-VL2 VL sequence; CDRs are underlined)
DIQLTQSPSSLSASVGDRVTITCKASQSVSNDVAWYQQKPGKAPKLLIYYASNRYTGVPSRFSGSGSGTD
FTFTISSLQPEDFATYYCQQDYSSPLTFGQGTKVEIK
SEQ ID NO: 87 (5F7D7-VH3 VH sequence; CDRs are underlined)
QVQLVQSGAEVKKPGASVKVSCKASGYTFTNYYMHWVRQAPGQRLEWIGYIYPNNGDTSYNQKFKG
RVTITVDTSASTAYMELSSLRSEDTAVYYCAVVSYSNYVAGAMDYWGQGTLVTVSS
SEQ ID NO: 88 (5F7D7-VL5 VL sequence; CDRs are underlined)
DIQMTQSPSSLSASVGDRVTITCKASQSVSNDVAWYQQKPGKAPKLLIYFASNRYTGVPSRFSGSGYGT
DFTFTISSLQPEDIATYFCQQDYSSPYTFGGGTKLEIK
SEQ ID NO: 89 (7F10B2-VH3 VH sequence; CDRs are underlined)
QVQLVQSGAEVKKPGASVKVSCKASGYTFTDYNMDWVRQAPGQGLEWMGTINPKNGETSDNQKFK
ARVTMTRDTSISTAYMELSRLRSDDTAVYYCARNVDYSNYLFFPMDYWGQGTLVTVSS
SEQ ID NO: 90 (7F10B2-VH5 VH sequence; CDRs are underlined)
QVQLVQSGAEVKKPGASVKVSCKASGYTFTDYNMDWVRQAPGQGLEWMGTINPKNGETSDNQKFK
ARVTVTTDTSTSTAYMELRSLRSDDTAVYYCARNVDYSNYLFFPMDYWGQGTTVTVSS
SEQ ID NO: 91 (7F10B2-VL1 VL sequence; CDRs are underlined)
DIQMTQSPSSLSASVGDRVTITCKASQSVSNDVAWYQQKPGKAPKLLIYYASNRFTGVPSRFSGSGSGTD
FTFTISSLQPEDIATYYCQQDYSSRTFGGGTKVEIK
SEQ ID NO: 92 (7F10B2-VL3 VL sequence; CDRs are underlined)
DIQMTQSPSSLSASVGDRVTITCKASQSVSNDVAWYQQKPGKAPKLLIYYASNRFTGVPSRFSGSGSGTD
FTLTISSLQPEDFATYYCQQDYSSRTFGGGTKVEIK
SEQ ID NO: 93 (31E2D4-VH4 VH sequence; CDRs are underlined)
QVQLVQSGAEVKKPGASVKVSCKASGYTLTNYGMNWVRQATGQGLEWMGWINSYSGVPTYADDFK
GRVTMTRNTSISTAYMELSSLRSEDTAVYYCARGENNYYGGSYDWGQGTTVTVSS
SEQ ID NO: 94 (42C3A4-VH1 VH sequence; CDRs are underlined)
EVQLVESGGGLVQPGGSLKVSCAASGITFSNYWMDWVRQASGKGLEWVGEIRLKSNNYATHYAESVK
GRFTISRDDSKNTAYLQMNSLKTEDTAVYYCTRGAPLFGGYYKGVYFDYWGQGTLVTVSS
SEQ ID NO: 95 (42C3A4-VL1 VL sequence; CDRs are underlined)
DIQMTQSPSSLSASVGDRVTITCQASQSVSNEVAWYQQKPGKAPKLLIYYASSRYTGVPSRFSGSGSGTD
FTFTISSLQPEDIATYYCQQDYNSPYTFGQGTKLEIK
SEQ ID NO: 96 (hABTAA-VH VH sequence; CDRs are underlined)
QVQLQESGPGLVKPSETLSLTCAVSGYSITSDYAWNWIRQPPGKGLEWMGKISYSGKTDYNPSLKSRSTI
SRDTSKNQFSLKLSSVTAADTAVYYCARGNFEGAMDYWGQGTLVTVSS
SEQ ID NO: 97 (hABTAA-VL VL sequence; CDRs are underlined)
DIQMTQSPSSLSASVGDRVTITCKASQSVSNDVAWYQQKPGKAPKLLIYYASNRYPGVPSRFSGSGSGT
DFTLTISSLQPEDFATYYCQQDYSSPWTFGQGTKLEIK
SEQ ID NO: 98 (5F7D7-VL2 VL sequence; CDRs are underlined)
DIQLTQSPSSLSASVGDRVTITCKASQSVSNDVAWYQQKPGKAPKLLIYFASNRYTGVPSRFSGSGSGTD
FTFTISSLQPEDFATYYCQQDYSSPYTFGQGTKVEIK
SEQ ID NO: 99 (Fc fragment sequence)
ASTKGPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRTPEVTC
VVVDVSQEDPEVQFNWYVDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLGK
SEQ ID NO: 100 (CL sequence)
RTVAAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
SEQ ID NO: 101 (Flt1d2 sequence)
SDTGRPFVEMYSEIPEIIHMTEGRELVIPCRVTSPNITVTLKKFPLDTLIPDGKRIIWDSRKGFIISNATYKEI
GLLTCEATVNGHLYKTNYLTHRQTNTII
SEQ ID NO: 102 (Flk1d3 sequence)
DVVLSPSHGIELSVGEKLVLNCTARTELNVGIDFNWEYPSSKHQHKKLVNRDLKTQSGSEMKKFLSTLTI
DGVTRSDQGLYTCAASSGLMTKKNSTFVRVHEK
SEQ ID NO: 103 (linker amino acid sequence, n is an integer of at least one)
(G)ₙ
SEQ ID NO: 104 (linker amino acid sequence, n is an integer of at least one)
(GS)ₙ
SEQ ID NO: 105 (linker amino acid sequence, n is an integer of at least one)
(GSGGS)ₙ
SEQ ID NO: 106 (linker amino acid sequence, n is an integer of at least one)
(GGGGS)ₙ
SEQ ID NO: 107 (linker amino acid sequence, n is an integer of at least one)
(GGGS)ₙ

1. An antibody fusion protein comprising: i) a multivalent antibody or antigen-binding fragment thereof specifically recognizing Angiopoietin-2 (Ang2)(“multivalent anti-Ang2 antibody or antigen-binding fragment thereof”), and ii) a vascular endothelial growth factor receptor (VEGFR) component, wherein the multivalent anti-Ang2 antibody or antigen-binding fragment thereof does not inhibit the binding between Ang2 and TEK receptor tyrosine kinase (TIE2).

2. The antibody fusion protein of claim 1, wherein the VEGFR component comprises an immunoglobulin (Ig)-like domain of one or more VEGFRs independently selected from the group consisting of Flt1, Flk1, and Flt4.

3. The antibody fusion protein of claim 1, wherein the VEGFR component comprises an Ig-like domain 2 of a first VEGFR and an Ig-like domain 3 of a second VEGFR, wherein the first VEGFR is Flt1, and the second VEGFR is Flk1 or Flt4.

4. (canceled)

5. The antibody fusion protein of claim 1, wherein the VEGFR component comprises an Ig-like domain 2 of Flt1 (Flt1d2) and an Ig-like domain 3 of Flk1 (Flk1d3).

6. The antibody fusion protein of claim 1, wherein the VEGFR component comprises an amino acid sequence of SEQ ID NO: 32.

7. The antibody fusion protein of claim 1, wherein the VEGFR component is fused to the multivalent anti-Ang2 antibody or antigen-binding fragment thereof via an optional linker.

8. (canceled)

9. The antibody fusion protein of claim 1, wherein the multivalent anti-Ang2 antibody or antigen-binding fragment thereof upon binding to Ang2 activates TIE2 signaling through the antibody-bound Ang2; wherein the multivalent anti-Ang2 antibody or antigen-binding fragment thereof comprises any of the following:
(1) i) an HC-CDR1 comprising the amino acid sequence of SEQ ID NO: 3; ii) an HC-CDR2 comprising the amino acid sequence of SEQ ID NO: 9; iii) an HC-CDR3 comprising the amino acid sequence of SEQ ID NO: 14; iv) an LC-CDR1 comprising the amino acid sequence of SEQ ID NO: 17; v) an LC-CDR2 comprising the amino acid sequence of SEQ ID NO: 21; and vi) an LC-CDR3 comprising the amino acid sequence of SEQ ID NO: 26; (2) i) an HC-CDR1 comprising the amino acid sequence of SEQ ID NO: 1; ii) an HC-CDR2 comprising the amino acid sequence of SEQ ID NO: 7; iii) an HC-CDR3 comprising the amino acid sequence of SEQ ID NO: 12; iv) an LC-CDR1 comprising the amino acid sequence of SEQ ID NO: 17; v) an LC-CDR2 comprising the amino acid sequence of SEQ ID NO: 19; and vi) an LC-CDR3 comprising the amino acid sequence of SEQ ID NO: 24; (3) i) an HC-CDR1 comprising the amino acid sequence of SEQ ID NO: 2; ii) an HC-CDR2 comprising the amino acid sequence of SEQ ID NO: 8; iii) an HC-CDR3 comprising the amino acid sequence of SEQ ID NO: 13; iv) an LC-CDR1 comprising the amino acid sequence of SEQ ID NO: 17; v) an LC-CDR2 comprising the amino acid sequence of SEQ ID NO: 20; and vi) an LC-CDR3 comprising the amino acid sequence of SEQ ID NO: 25; (4) i) an HC-CDR1 comprising the amino acid sequence of SEQ ID NO: 4; ii) an HC-CDR2 comprising the amino acid sequence of SEQ ID NO: 7; iii) an HC-CDR3 comprising the amino acid sequence of SEQ ID NO: 12; iv) an LC-CDR1 comprising the amino acid sequence of SEQ ID NO: 17; v) an LC-CDR2 comprising the amino acid sequence of SEQ ID NO: 19; and vi) an LC-CDR3 comprising the amino acid sequence of SEQ ID NO: 24; (5) i) an HC-CDR1 comprising the amino acid sequence of SEQ ID NO: 5; ii) an HC-CDR2 comprising the amino acid sequence of SEQ ID NO: 10; iii) an HC-CDR3 comprising the amino acid sequence of SEQ ID NO: 15; iv) an LC-CDR1 comprising the amino acid sequence of SEQ ID NO: 18; v) an LC-CDR2 comprising the amino acid sequence of SEQ ID NO: 22; and vi) an LC-CDR3 comprising the amino acid sequence of SEQ ID NO: 27; or
(6) i) an HC-CDR1 comprising the amino acid sequence of SEQ ID NO: 6; ii) an HC-CDR2 comprising the amino acid sequence of SEQ ID NO: 11; iii) an HC-CDR3 comprising the amino acid sequence of SEQ ID NO: 16; iv) an LC-CDR1 comprising the amino acid sequence of SEQ ID NO: 17; v) an LC-CDR2 comprising the amino acid sequence of SEQ ID NO: 23; and vi) an LC-CDR3 comprising the amino acid sequence of SEQ ID NO: 28.

10. The antibody fusion protein of claim 1, wherein the multivalent anti-Ang2 antibody or antigen-binding fragment thereof is a full length antibody (“anti-Ang2 full length antibody”), wherein the anti-Ang2 full length antibody comprises any of the following:
(1) a heavy chain comprising the amino acid sequence of SEQ ID NO: 36 or 37, and a light chain comprising the amino acid sequence of SEQ ID NO: 45 or 46;
(2) a heavy chain comprising the amino acid sequence of SEQ ID NO: 33 or 34, and a light chain comprising the amino acid sequence of any of SEQ ID NOs: 41-43;
(3) a heavy chain comprising the amino acid sequence of SEQ ID NO: 35, and a light chain comprising the amino acid sequence of SEQ ID NO: 44 or 49;
(4) a heavy chain comprising the amino acid sequence of SEQ ID NO: 38, and a light chain comprising the amino acid sequence of any of SEQ ID NOs: 41-43;
(5) a heavy chain comprising the amino acid sequence of SEQ ID NO: 39, and a light chain comprising the amino acid sequence of SEQ ID NO: 47; or
(6) a heavy chain comprising the amino acid sequence of SEQ ID NO: 40, and a light chain comprising the amino acid sequence of SEQ ID NO: 48.

11.-14. (canceled)

15. The antibody fusion protein of claim 10, wherein the VEGFR component is fused to the N-terminus of a heavy chain of the anti-Ang2 full length antibody via an optional linker.

16. (canceled)

17. The antibody fusion protein of claim 15, wherein the antibody fusion protein comprises any of the following:
(1) a heavy chain fusion polypeptide comprising the amino acid sequence of SEQ ID NO: 56 or 58, and a light chain comprising the amino acid sequence of SEQ ID NO: 42;
(2) a heavy chain fusion polypeptide comprising the amino acid sequence of SEQ ID NO: 57, and a light chain comprising the amino acid sequence of SEQ ID NO: 41 or 42;
(3) a heavy chain fusion polypeptide comprising the amino acid sequence of SEQ ID NO: 59, and a light chain comprising the amino acid sequence of SEQ ID NO: 45;
(4) a heavy chain fusion polypeptide comprising the amino acid sequence of SEQ ID NO: 60, and a light chain comprising the amino acid sequence of SEQ ID NO: 46;
(5) a heavy chain fusion polypeptide comprising the amino acid sequence of SEQ ID NO: 61, and a light chain comprising the amino acid sequence of SEQ ID NO: 44;
(6) a heavy chain fusion polypeptide comprising the amino acid sequence of SEQ ID NO: 75, and a light chain comprising the amino acid sequence of SEQ ID NO: 47; or
(7) a heavy chain fusion polypeptide comprising the amino acid sequence of SEQ ID NO: 79, and a light chain comprising the amino acid sequence of SEQ ID NO: 48.

18.-19. (canceled)

20. The antibody fusion protein of claim 10, wherein the VEGFR component is fused to the C-terminus of a heavy chain of the anti-Ang2 full length antibody via an optional linker.

21. The antibody fusion protein of claim 20, wherein the obtained heavy chain fusion polypeptide comprises the amino acid sequence of any of SEQ ID NOs: 50-55, 74, and 78.

22. The antibody fusion protein of claim 20, wherein the antibody fusion protein comprises any of the following:
(1) a heavy chain fusion polypeptide comprising the amino acid sequence of SEQ ID NO: 54, and a light chain comprising the amino acid sequence of SEQ ID NO: 46;
(2) a heavy chain fusion polypeptide comprising the amino acid sequence of SEQ ID NO: 50 or 52, and a light chain comprising the amino acid sequence of SEQ ID NO: 42;
(3) a heavy chain fusion polypeptide comprising the amino acid sequence of SEQ ID NO: 51, and a light chain comprising the amino acid sequence of SEQ ID NO: 41 or 42;
(4) a heavy chain fusion polypeptide comprising the amino acid sequence of SEQ ID NO: 53, and a light chain comprising the amino acid sequence of SEQ ID NO: 45;
(5) a heavy chain fusion polypeptide comprising the amino acid sequence of SEQ ID NO: 55, and a light chain comprising the amino acid sequence of SEQ ID NO: 44;
(6) a heavy chain fusion polypeptide comprising the amino acid sequence of SEQ ID NO: 74, and a light chain comprising the amino acid sequence of SEQ ID NO: 47; or
(7) a heavy chain fusion polypeptide comprising the amino acid sequence of SEQ ID NO: 78, and a light chain comprising the amino acid sequence of SEQ ID NO: 48.

23. The antibody fusion protein of claim 20, wherein the antibody fusion protein comprises a first VEGFR component and a second VEGFR component, wherein the first VEGFR component is fused to the C-terminus of a first heavy chain of the anti-Ang2 full length antibody via an optional first linker, and wherein the second VEGFR component is fused to the C-terminus of a second heavy chain of the anti-Ang2 full length antibody via an optional second linker.

24. (canceled)

25. The antibody fusion protein of claim 10, wherein the VEGFR component is fused to the N-terminus of a light chain of the anti-Ang2 full length antibody via an optional linker; or
the VEGFR component is fused to the C-terminus of a light chain of the anti-Ang2 full length antibody via an optional linker.

26.-34. (canceled)

35. The antibody fusion protein of claim 10, wherein the antibody fusion protein comprises four VEGFR components, wherein the antibody fusion protein comprises any of the following:
(1) wherein the first VEGFR component is fused to the N-terminus of a first heavy chain of the anti-Ang2 full length antibody via an optional first linker, wherein the second VEGFR component is fused to the N-terminus of a second heavy chain of the anti-Ang2 full length antibody via an optional second linker, wherein the third VEGFR component is fused to the N-terminus of a first light chain of the anti-Ang2 full length antibody via an optional third linker, and wherein the fourth VEGFR component is fused to the N-terminus of a second light chain of the anti-Ang2 full length antibody via an optional fourth linker;
(2) wherein the first VEGFR component is fused to the C-terminus of a first heavy chain of the anti-Ang2 full length antibody via an optional first linker, wherein the second VEGFR component is fused to the C-terminus of a second heavy chain of the anti-Ang2 full length antibody via an optional second linker, wherein the third VEGFR component is fused to the C-terminus of a first light chain of the anti-Ang2 full length antibody via an optional third linker, and wherein the fourth VEGFR component is fused to the C-terminus of a second light chain of the anti-Ang2 full length antibody via an optional fourth linker;
(3) wherein the first VEGFR component is fused to the N-terminus of a first heavy chain of the anti-Ang2 full length antibody via an optional first linker, wherein the second VEGFR component is fused to the N-terminus of a second heavy chain of the anti-Ang2 full length antibody via an optional second linker, wherein the third VEGFR component is fused to the C-terminus of the first heavy chain of the anti-Ang2 full length antibody via an optional third linker, and wherein the fourth VEGFR component is fused to the C-terminus of the second heavy chain of the anti-Ang2 full length antibody via an optional fourth linker;
(4) wherein the first VEGFR component is fused to the C-terminus of a first heavy chain of the anti-Ang2 full length antibody via an optional first linker, wherein the second VEGFR component is fused to the C-terminus of a second heavy chain of the anti-Ang2 full length antibody via an optional second linker, wherein the third VEGFR component is fused to the N-terminus of a first light chain of the anti-Ang2 full length antibody via an optional third linker, and wherein the fourth VEGFR component is fused to the N-terminus of a second light chain of the anti-Ang2 full length antibody via an optional fourth linker;
(5) wherein the first VEGFR component is fused to the N-terminus of a first heavy chain of the anti-Ang2 full length antibody via an optional first linker, wherein the second VEGFR component is fused to the N-terminus of a second heavy chain of the anti-Ang2 full length antibody via an optional second linker, wherein the third VEGFR component is fused to the C-terminus of a first light chain of the anti-Ang2 full length antibody via an optional third linker, and wherein the fourth VEGFR component is fused to the C-terminus of a second light chain of the anti-Ang2 full length antibody via an optional fourth linker; or
(6) wherein the first VEGFR component is fused to the N-terminus of a first light chain of the anti-Ang2 full length antibody via an optional first linker, wherein the second VEGFR component is fused to the N-terminus of a second light chain of the anti-Ang2 full length antibody via an optional second linker, wherein the third VEGFR component is fused to the C-terminus of the first light chain of the anti-Ang2 full length antibody via an optional third linker, and wherein the fourth VEGFR component is fused to the C-terminus of the second light chain of the anti-Ang2 full length antibody via an optional fourth linker.

36.-40. (canceled)

41. An isolated nucleic acid encoding the antibody fusion protein of claim 1.

42. A vector comprising the isolated nucleic acid of claim 41.

43.-46. (canceled)

47. A pharmaceutical composition comprising the antibody fusion protein of claim 1, and a pharmaceutical acceptable carrier.

48. A method of treating a cancer in an individual, comprising administering to the individual an effective amount of the antibody fusion protein of claim 1.

49.-51. (canceled)

52. A method of treating a non-neoplastic disorder in an individual, comprising administering to the individual an effective amount of the antibody fusion protein of claim 1.

53.-56. (canceled)

57. The method of claim 52, wherein the non-neoplastic disorder is an ocular neovascular disorder; wherein the ocular neovascular disorder is age-related macular degeneration (AMD) or diabetic retinopathy or is associated with one or more of choroidal neovascularization, vascular leak, and retinal edema.

58.-61. (canceled)


SEQUENCE LISTING (107 sequences)

SEQ ID NO: 1 - protein, 5 aa, Artificial Sequence
  Synthetic Construct
NYGVN

SEQ ID NO: 2 - protein, 5 aa, Artificial Sequence
  Synthetic Construct
NYYMH

SEQ ID NO: 3 - protein, 5 aa, Artificial Sequence
  Synthetic Construct
DYNMD

SEQ ID NO: 4 - protein, 5 aa, Artificial Sequence
  Synthetic Construct
NYGMN

SEQ ID NO: 5 - protein, 5 aa, Artificial Sequence
  Synthetic Construct
NYWMD

SEQ ID NO: 6 - protein, 6 aa, Artificial Sequence
  Synthetic Construct
SDYAWN

SEQ ID NO: 7 - protein, 17 aa, Artificial Sequence
  Synthetic Construct
WINSYSGVPTYADDFKG

SEQ ID NO: 8 - protein, 17 aa, Artificial Sequence
  Synthetic Construct
YIYPNNGDTSYNQKFKG

SEQ ID NO: 9 - protein, 17 aa, Artificial Sequence
  Synthetic Construct
TINPKNGETSDNQKFKA

SEQ ID NO: 10 - protein, 19 aa, Artificial Sequence
  Synthetic Construct
EIRLKSNNYATHYAESVKG

SEQ ID NO: 11 - protein, 16 aa, Artificial Sequence
  Synthetic Construct
KISYSGKTDYNPSLKS

SEQ ID NO: 12 - protein, 11 aa, Artificial Sequence
  Synthetic Construct
GENNYYGGSYD

SEQ ID NO: 13 - protein, 13 aa, Artificial Sequence
  Synthetic Construct
VSYSNYVAGAMDY

SEQ ID NO: 14 - protein, 14 aa, Artificial Sequence
  Synthetic Construct
NVDYSNYLFFPMDY

SEQ ID NO: 15 - protein, 16 aa, Artificial Sequence
  Synthetic Construct
GAPLFGGYYKGVYFDY

SEQ ID NO: 16 - protein, 9 aa, Artificial Sequence
  Synthetic Construct
GNFEGAMDY

SEQ ID NO: 17 - protein, 11 aa, Artificial Sequence
  Synthetic Construct
KASQSVSNDVA

SEQ ID NO: 18 - protein, 11 aa, Artificial Sequence
  Synthetic Construct
QASQSVSNEVA

SEQ ID NO: 19 - protein, 7 aa, Artificial Sequence
  Synthetic Construct
YASNRYT

SEQ ID NO: 20 - protein, 7 aa, Artificial Sequence
  Synthetic Construct
FASNRYT

SEQ ID NO: 21 - protein, 7 aa, Artificial Sequence
  Synthetic Construct
YASNRFT

SEQ ID NO: 22 - protein, 7 aa, Artificial Sequence
  Synthetic Construct
YASSRYT

SEQ ID NO: 23 - protein, 7 aa, Artificial Sequence
  Synthetic Construct
YASNRYP

SEQ ID NO: 24 - protein, 9 aa, Artificial Sequence
  Synthetic Construct
QQDYSSPLT

SEQ ID NO: 25 - protein, 9 aa, Artificial Sequence
  Synthetic Construct
QQDYSSPYT

SEQ ID NO: 26 - protein, 8 aa, Artificial Sequence
  Synthetic Construct
QQDYSSRT

SEQ ID NO: 27 - protein, 9 aa, Artificial Sequence
  Synthetic Construct
QQDYNSPYT

SEQ ID NO: 28 - protein, 9 aa, Artificial Sequence
  Synthetic Construct
QQDYSSPWT

SEQ ID NO: 29 - protein, 19 aa, Artificial Sequence
  Synthetic Construct
MGWSCIILFLVATATGVHS

SEQ ID NO: 30 - protein, 15 aa, Artificial Sequence
  Synthetic Construct
GGGGSGGGGSGGGGS

SEQ ID NO: 31 - protein, 12 aa, Artificial Sequence
  Synthetic Construct
ESKYGPPSPPSP

SEQ ID NO: 32 - protein, 205 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 33 - protein, 447 aa, Artificial Sequence
  Synthetic Construct
QVQLVQSGAEVKKPGASVKVSCKASGYTFTNYGVNWVRQAPGQRLEWMGWINSYSGVPTYADDFKGRFTITRDTSASTAYMELSSLRSEDTAVYYCARGENNYYGGSYDWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 447 aa, Artificial Sequence
  Synthetic Construct
QVQLVQSGAEVKKPGASVKVSCKASGYTFTNYGVNWVRQAPGQRLEWMGWINSYSGVPTYADDFKGRVTITRDTSASTAYMELSSLRSEDTAVYYCARGENNYYGGSYDWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 449 aa, Artificial Sequence
  Synthetic Construct
QVQLVQSGAEVKKPGASVKVSCKASGYTFTNYYMHWVRQAPGQRLEWIGYIYPNNGDTSYNQKFKGRVTITVDTSASTAYMELSSLRSEDTAVYYCAVVSYSNYVAGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 450 aa, Artificial Sequence
  Synthetic Construct
QVQLVQSGAEVKKPGASVKVSCKASGYTFTDYNMDWVRQAPGQGLEWMGTINPKNGETSDNQKFKARVTMTRDTSISTAYMELSRLRSDDTAVYYCARNVDYSNYLFFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 450 aa, Artificial Sequence
  Synthetic Construct
QVQLVQSGAEVKKPGASVKVSCKASGYTFTDYNMDWVRQAPGQGLEWMGTINPKNGETSDNQKFKARVTVTTDTSTSTAYMELRSLRSDDTAVYYCARNVDYSNYLFFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 447 aa, Artificial Sequence
  Synthetic Construct
QVQLVQSGAEVKKPGASVKVSCKASGYTLTNYGMNWVRQATGQGLEWMGWINSYSGVPTYADDFKGRVTMTRNTSISTAYMELSSLRSEDTAVYYCARGENNYYGGSYDWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 454 aa, Artificial Sequence
  Synthetic Construct
EVQLVESGGGLVQPGGSLKVSCAASGITFSNYWMDWVRQASGKGLEWVGEIRLKSNNYATHYAESVKGRFTISRDDSKNTAYLQMNSLKTEDTAVYYCTRGAPLFGGYYKGVYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 445 aa, Artificial Sequence
  Synthetic Construct
QVQLQESGPGLVKPSETLSLTCAVSGYSITSDYAWNWIRQPPGKGLEWMGKISYSGKTDYNPSLKSRSTISRDTSKNQFSLKLSSVTAADTAVYYCARGNFEGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 214 aa, Artificial Sequence
  Synthetic Construct
DIQMTQSPSSLSASVGDRVTITCKASQSVSNDVAWYQQKPGKAPKLLIYYASNRYTGVPSRFSGSGSGTDFTLTISSLQPEDFATYFCQQDYSSPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 214 aa, Artificial Sequence
  Synthetic Construct
DIQMTQSPSSLSASVGDRVTITCKASQSVSNDVAWYQQKPGKAPKLLIYYASNRYTGVPSRFSGSGSGTDFTLTISSLQPEDFATYYCQQDYSSPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 214 aa, Artificial Sequence
  Synthetic Construct
DIQLTQSPSSLSASVGDRVTITCKASQSVSNDVAWYQQKPGKAPKLLIYYASNRYTGVPSRFSGSGSGTDFTFTISSLQPEDFATYYCQQDYSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 214 aa, Artificial Sequence
  Synthetic Construct
DIQMTQSPSSLSASVGDRVTITCKASQSVSNDVAWYQQKPGKAPKLLIYFASNRYTGVPSRFSGSGYGTDFTFTISSLQPEDIATYFCQQDYSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 213 aa, Artificial Sequence
  Synthetic Construct
DIQMTQSPSSLSASVGDRVTITCKASQSVSNDVAWYQQKPGKAPKLLIYYASNRFTGVPSRFSGSGSGTDFTFTISSLQPEDIATYYCQQDYSSRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 213 aa, Artificial Sequence
  Synthetic Construct
DIQMTQSPSSLSASVGDRVTITCKASQSVSNDVAWYQQKPGKAPKLLIYYASNRFTGVPSRFSGSGSGTDFTLTISSLQPEDFATYYCQQDYSSRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 214 aa, Artificial Sequence
  Synthetic Construct
DIQMTQSPSSLSASVGDRVTITCQASQSVSNEVAWYQQKPGKAPKLLIYYASSRYTGVPSRFSGSGSGTDFTFTISSLQPEDIATYYCQQDYNSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 214 aa, Artificial Sequence
  Synthetic Construct
DIQMTQSPSSLSASVGDRVTITCKASQSVSNDVAWYQQKPGKAPKLLIYYASNRYPGVPSRFSGSGSGTDFTLTISSLQPEDFATYYCQQDYSSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 214 aa, Artificial Sequence
  Synthetic Construct
DIQLTQSPSSLSASVGDRVTITCKASQSVSNDVAWYQQKPGKAPKLLIYFASNRYTGVPSRFSGSGSGTDFTFTISSLQPEDFATYYCQQDYSSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 667 aa, Artificial Sequence
  Synthetic Construct
QVQLVQSGAEVKKPGASVKVSCKASGYTFTNYGVNWVRQAPGQRLEWMGWINSYSGVPTYADDFKGRFTITRDTSASTAYMELSSLRSEDTAVYYCARGENNYYGGSYDWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 51 - protein, 667 aa, Artificial Sequence
  Synthetic Construct
QVQLVQSGAEVKKPGASVKVSCKASGYTFTNYGVNWVRQAPGQRLEWMGWINSYSGVPTYADDFKGRVTITRDTSASTAYMELSSLRSEDTAVYYCARGENNYYGGSYDWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 52 - protein, 667 aa, Artificial Sequence
  Synthetic Construct
QVQLVQSGAEVKKPGASVKVSCKASGYTLTNYGMNWVRQATGQGLEWMGWINSYSGVPTYADDFKGRVTMTRNTSISTAYMELSSLRSEDTAVYYCARGENNYYGGSYDWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 53 - protein, 670 aa, Artificial Sequence
  Synthetic Construct
QVQLVQSGAEVKKPGASVKVSCKASGYTFTDYNMDWVRQAPGQGLEWMGTINPKNGETSDNQKFKARVTVTTDTSTSTAYMELRSLRSDDTAVYYCARNVDYSNYLFFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 54 - protein, 670 aa, Artificial Sequence
  Synthetic Construct
QVQLVQSGAEVKKPGASVKVSCKASGYTFTDYNMDWVRQAPGQGLEWMGTINPKNGETSDNQKFKARVTMTRDTSISTAYMELSRLRSDDTAVYYCARNVDYSNYLFFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 55 - protein, 669 aa, Artificial Sequence
  Synthetic Construct
QVQLVQSGAEVKKPGASVKVSCKASGYTFTNYYMHWVRQAPGQRLEWIGYIYPNNGDTSYNQKFKGRVTITVDTSASTAYMELSSLRSEDTAVYYCAVVSYSNYVAGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 56 - protein, 667 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSQVQLVQSGAEVKKPGASVKVSCKASGYTFTNYGVNWVRQAPGQRLEWMGWINSYSGVPTYADDFKGRFTITRDTSASTAYMELSSLRSEDTAVYYCARGENNYYGGSYDWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 667 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSQVQLVQSGAEVKKPGASVKVSCKASGYTFTNYGVNWVRQAPGQRLEWMGWINSYSGVPTYADDFKGRVTITRDTSASTAYMELSSLRSEDTAVYYCARGENNYYGGSYDWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 667 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSQVQLVQSGAEVKKPGASVKVSCKASGYTLTNYGMNWVRQATGQGLEWMGWINSYSGVPTYADDFKGRVTMTRNTSISTAYMELSSLRSEDTAVYYCARGENNYYGGSYDWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 670 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSQVQLVQSGAEVKKPGASVKVSCKASGYTFTDYNMDWVRQAPGQGLEWMGTINPKNGETSDNQKFKARVTVTTDTSTSTAYMELRSLRSDDTAVYYCARNVDYSNYLFFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 670 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSQVQLVQSGAEVKKPGASVKVSCKASGYTFTDYNMDWVRQAPGQGLEWMGTINPKNGETSDNQKFKARVTMTRDTSISTAYMELSRLRSDDTAVYYCARNVDYSNYLFFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 669 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSQVQLVQSGAEVKKPGASVKVSCKASGYTFTNYYMHWVRQAPGQRLEWIGYIYPNNGDTSYNQKFKGRVTITVDTSASTAYMELSSLRSEDTAVYYCAVVSYSNYVAGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 431 aa, Artificial Sequence
  Synthetic Construct
DIQMTQSPSSLSASVGDRVTITCKASQSVSNDVAWYQQKPGKAPKLLIYYASNRYTGVPSRFSGSGSGTDFTLTISSLQPEDFATYYCQQDYSSPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPSPPSPSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 63 - protein, 431 aa, Artificial Sequence
  Synthetic Construct
DIQMTQSPSSLSASVGDRVTITCKASQSVSNDVAWYQQKPGKAPKLLIYYASNRYTGVPSRFSGSGSGTDFTLTISSLQPEDFATYFCQQDYSSPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPSPPSPSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 64 - protein, 430 aa, Artificial Sequence
  Synthetic Construct
DIQMTQSPSSLSASVGDRVTITCKASQSVSNDVAWYQQKPGKAPKLLIYYASNRFTGVPSRFSGSGSGTDFTFTISSLQPEDIATYYCQQDYSSRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPSPPSPSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 65 - protein, 430 aa, Artificial Sequence
  Synthetic Construct
DIQMTQSPSSLSASVGDRVTITCKASQSVSNDVAWYQQKPGKAPKLLIYYASNRFTGVPSRFSGSGSGTDFTLTISSLQPEDFATYYCQQDYSSRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPSPPSPSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 66 - protein, 431 aa, Artificial Sequence
  Synthetic Construct
DIQMTQSPSSLSASVGDRVTITCKASQSVSNDVAWYQQKPGKAPKLLIYFASNRYTGVPSRFSGSGYGTDFTFTISSLQPEDIATYFCQQDYSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPSPPSPSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 67 - protein, 434 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSDIQMTQSPSSLSASVGDRVTITCKASQSVSNDVAWYQQKPGKAPKLLIYYASNRYTGVPSRFSGSGSGTDFTLTISSLQPEDFATYYCQQDYSSPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 434 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSDIQMTQSPSSLSASVGDRVTITCKASQSVSNDVAWYQQKPGKAPKLLIYYASNRYTGVPSRFSGSGSGTDFTLTISSLQPEDFATYFCQQDYSSPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 433 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSDIQMTQSPSSLSASVGDRVTITCKASQSVSNDVAWYQQKPGKAPKLLIYYASNRFTGVPSRFSGSGSGTDFTFTISSLQPEDIATYYCQQDYSSRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 433 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSDIQMTQSPSSLSASVGDRVTITCKASQSVSNDVAWYQQKPGKAPKLLIYYASNRFTGVPSRFSGSGSGTDFTLTISSLQPEDFATYYCQQDYSSRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 434 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSDIQMTQSPSSLSASVGDRVTITCKASQSVSNDVAWYQQKPGKAPKLLIYFASNRYTGVPSRFSGSGYGTDFTFTISSLQPEDIATYFCQQDYSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 431 aa, Artificial Sequence
  Synthetic Construct
DIQMTQSPSSLSASVGDRVTITCQASQSVSNEVAWYQQKPGKAPKLLIYYASSRYTGVPSRFSGSGSGTDFTFTISSLQPEDIATYYCQQDYNSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPSPPSPSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 73 - protein, 434 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSDIQMTQSPSSLSASVGDRVTITCQASQSVSNEVAWYQQKPGKAPKLLIYYASSRYTGVPSRFSGSGSGTDFTFTISSLQPEDIATYYCQQDYNSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 674 aa, Artificial Sequence
  Synthetic Construct
EVQLVESGGGLVQPGGSLKVSCAASGITFSNYWMDWVRQASGKGLEWVGEIRLKSNNYATHYAESVKGRFTISRDDSKNTAYLQMNSLKTEDTAVYYCTRGAPLFGGYYKGVYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 75 - protein, 674 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSEVQLVESGGGLVQPGGSLKVSCAASGITFSNYWMDWVRQASGKGLEWVGEIRLKSNNYATHYAESVKGRFTISRDDSKNTAYLQMNSLKTEDTAVYYCTRGAPLFGGYYKGVYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 431 aa, Artificial Sequence
  Synthetic Construct
DIQLTQSPSSLSASVGDRVTITCKASQSVSNDVAWYQQKPGKAPKLLIYYASNRYTGVPSRFSGSGSGTDFTFTISSLQPEDFATYYCQQDYSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPSPPSPSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 77 - protein, 434 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSDIQLTQSPSSLSASVGDRVTITCKASQSVSNDVAWYQQKPGKAPKLLIYYASNRYTGVPSRFSGSGSGTDFTFTISSLQPEDFATYYCQQDYSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 665 aa, Artificial Sequence
  Synthetic Construct
QVQLQESGPGLVKPSETLSLTCAVSGYSITSDYAWNWIRQPPGKGLEWMGKISYSGKTDYNPSLKSRSTISRDTSKNQFSLKLSSVTAADTAVYYCARGNFEGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 79 - protein, 665 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSQVQLQESGPGLVKPSETLSLTCAVSGYSITSDYAWNWIRQPPGKGLEWMGKISYSGKTDYNPSLKSRSTISRDTSKNQFSLKLSSVTAADTAVYYCARGNFEGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 431 aa, Artificial Sequence
  Synthetic Construct
DIQMTQSPSSLSASVGDRVTITCKASQSVSNDVAWYQQKPGKAPKLLIYYASNRYPGVPSRFSGSGSGTDFTLTISSLQPEDFATYYCQQDYSSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPSPPSPSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 81 - protein, 434 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSDIQMTQSPSSLSASVGDRVTITCKASQSVSNDVAWYQQKPGKAPKLLIYYASNRYPGVPSRFSGSGSGTDFTLTISSLQPEDFATYYCQQDYSSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 120 aa, Artificial Sequence
  Synthetic Construct
QVQLVQSGAEVKKPGASVKVSCKASGYTFTNYGVNWVRQAPGQRLEWMGWINSYSGVPTYADDFKGRFTITRDTSASTAYMELSSLRSEDTAVYYCARGENNYYGGSYDWGQGTLVTVSS

SEQ ID NO: 83 - protein, 120 aa, Artificial Sequence
  Synthetic Construct
QVQLVQSGAEVKKPGASVKVSCKASGYTFTNYGVNWVRQAPGQRLEWMGWINSYSGVPTYADDFKGRVTITRDTSASTAYMELSSLRSEDTAVYYCARGENNYYGGSYDWGQGTLVTVSS

SEQ ID NO: 84 - protein, 107 aa, Artificial Sequence
  Synthetic Construct
DIQMTQSPSSLSASVGDRVTITCKASQSVSNDVAWYQQKPGKAPKLLIYYASNRYTGVPSRFSGSGSGTDFTLTISSLQPEDFATYFCQQDYSSPLTFGGGTKLEIK

SEQ ID NO: 85 - protein, 107 aa, Artificial Sequence
  Synthetic Construct
DIQMTQSPSSLSASVGDRVTITCKASQSVSNDVAWYQQKPGKAPKLLIYYASNRYTGVPSRFSGSGSGTDFTLTISSLQPEDFATYYCQQDYSSPLTFGGGTKLEIK

SEQ ID NO: 86 - protein, 107 aa, Artificial Sequence
  Synthetic Construct
DIQLTQSPSSLSASVGDRVTITCKASQSVSNDVAWYQQKPGKAPKLLIYYASNRYTGVPSRFSGSGSGTDFTFTISSLQPEDFATYYCQQDYSSPLTFGQGTKVEIK

SEQ ID NO: 87 - protein, 122 aa, Artificial Sequence
  Synthetic Construct
QVQLVQSGAEVKKPGASVKVSCKASGYTFTNYYMHWVRQAPGQRLEWIGYIYPNNGDTSYNQKFKGRVTITVDTSASTAYMELSSLRSEDTAVYYCAVVSYSNYVAGAMDYWGQGTLVTVSS

SEQ ID NO: 88 - protein, 107 aa, Artificial Sequence
  Synthetic Construct
DIQMTQSPSSLSASVGDRVTITCKASQSVSNDVAWYQQKPGKAPKLLIYFASNRYTGVPSRFSGSGYGTDFTFTISSLQPEDIATYFCQQDYSSPYTFGGGTKLEIK

SEQ ID NO: 89 - protein, 123 aa, Artificial Sequence
  Synthetic Construct
QVQLVQSGAEVKKPGASVKVSCKASGYTFTDYNMDWVRQAPGQGLEWMGTINPKNGETSDNQKFKARVTMTRDTSISTAYMELSRLRSDDTAVYYCARNVDYSNYLFFPMDYWGQGTLVTVSS

SEQ ID NO: 90 - protein, 123 aa, Artificial Sequence
  Synthetic Construct
QVQLVQSGAEVKKPGASVKVSCKASGYTFTDYNMDWVRQAPGQGLEWMGTINPKNGETSDNQKFKARVTVTTDTSTSTAYMELRSLRSDDTAVYYCARNVDYSNYLFFPMDYWGQGTTVTVSS

SEQ ID NO: 91 - protein, 106 aa, Artificial Sequence
  Synthetic Construct
DIQMTQSPSSLSASVGDRVTITCKASQSVSNDVAWYQQKPGKAPKLLIYYASNRFTGVPSRFSGSGSGTDFTFTISSLQPEDIATYYCQQDYSSRTFGGGTKVEIK

SEQ ID NO: 92 - protein, 106 aa, Artificial Sequence
  Synthetic Construct
DIQMTQSPSSLSASVGDRVTITCKASQSVSNDVAWYQQKPGKAPKLLIYYASNRFTGVPSRFSGSGSGTDFTLTISSLQPEDFATYYCQQDYSSRTFGGGTKVEIK

SEQ ID NO: 93 - protein, 120 aa, Artificial Sequence
  Synthetic Construct
QVQLVQSGAEVKKPGASVKVSCKASGYTLTNYGMNWVRQATGQGLEWMGWINSYSGVPTYADDFKGRVTMTRNTSISTAYMELSSLRSEDTAVYYCARGENNYYGGSYDWGQGTTVTVSS

SEQ ID NO: 94 - protein, 127 aa, Artificial Sequence
  Synthetic Construct
EVQLVESGGGLVQPGGSLKVSCAASGITFSNYWMDWVRQASGKGLEWVGEIRLKSNNYATHYAESVKGRFTISRDDSKNTAYLQMNSLKTEDTAVYYCTRGAPLFGGYYKGVYFDYWGQGTLVTVSS

SEQ ID NO: 95 - protein, 107 aa, Artificial Sequence
  Synthetic Construct
DIQMTQSPSSLSASVGDRVTITCQASQSVSNEVAWYQQKPGKAPKLLIYYASSRYTGVPSRFSGSGSGTDFTFTISSLQPEDIATYYCQQDYNSPYTFGQGTKLEIK

SEQ ID NO: 96 - protein, 118 aa, Artificial Sequence
  Synthetic Construct
QVQLQESGPGLVKPSETLSLTCAVSGYSITSDYAWNWIRQPPGKGLEWMGKISYSGKTDYNPSLKSRSTISRDTSKNQFSLKLSSVTAADTAVYYCARGNFEGAMDYWGQGTLVTVSS

SEQ ID NO: 97 - protein, 107 aa, Artificial Sequence
  Synthetic Construct
DIQMTQSPSSLSASVGDRVTITCKASQSVSNDVAWYQQKPGKAPKLLIYYASNRYPGVPSRFSGSGSGTDFTLTISSLQPEDFATYYCQQDYSSPWTFGQGTKLEIK

SEQ ID NO: 98 - protein, 107 aa, Artificial Sequence
  Synthetic Construct
DIQLTQSPSSLSASVGDRVTITCKASQSVSNDVAWYQQKPGKAPKLLIYFASNRYTGVPSRFSGSGSGTDFTFTISSLQPEDFATYYCQQDYSSPYTFGQGTKVEIK

SEQ ID NO: 99 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - protein, 107 aa, Homo sapiens [sequence omitted]

SEQ ID NO: 101 - protein, 102 aa, Homo sapiens
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTII

SEQ ID NO: 102 - protein, 103 aa, Homo sapiens
DVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 103 - protein, 1 aa, Artificial Sequence
  Synthetic Construct
  VARIANT 1: Can be present in repeats of any integer
G

SEQ ID NO: 104 - protein, 2 aa, Artificial Sequence
  Synthetic Construct
  VARIANT (1)...(2): Can be present in repeats of any integer
GS

SEQ ID NO: 105 - protein, 5 aa, Artificial Sequence
  Synthetic Construct
  VARIANT (1)...(5): Can be present in repeats of any integer
GSGGS

SEQ ID NO: 106 - protein, 5 aa, Artificial Sequence
  Synthetic Construct
  VARIANT (1)...(5): Can be present in repeats of any integer
GGGGS

SEQ ID NO: 107 - protein, 4 aa, Artificial Sequence
  Synthetic Construct
  VARIANT (1)...(4): Can be present in repeats of any integer
GGGS